(19) United States
(12) Patent Application Publication
(10) Pub. No.: US 2005/0064563 A1
(43) Pub. Date: Mar. 24, 2005

(54) Nucleic acids for aminocoumarin biosynthesis

(76) Inventors: Lutz Heide, Tubingen (DE); Shu-Ming Li, Tubingen (DE)

Correspondence Address: SUGHRUE MION, PLLC, 2100 PENNSYLVANIA AVENUE, N.W., SUITE 800, WASHINGTON, DC, 20037, US

(21) Appl. No.: 10/485,710

(22) PCT Filed: Aug. 6, 2002
(86) PCT No.: PCT/EP02/08777

Related U.S. Application Data
(60) Provisional application No. 60/310,808, filed on Aug. 8, 2001.

(51) Int. Cl. C12P 17/06; C12N 9/10; C07H 21/04; C07D 311/02; C12N 15/74

(52) U.S. Cl. 435/125; 435/69.1; 435/252.3; 435/320.1; 536/23.2; 549/288; 435/193

(57) ABSTRACT

The present invention relates to isolated nucleic acids coding for enzymes or functionally active fragments thereof encoded by aminocoumarin biosynthetic gene clusters, to novel aminocoumarin compounds as well as to a method for the production of modified aminocoumarins utilizing the genetic information contained in said aminocoumarin biosynthetic gene clusters.

The present invention relates to isolated nucleic acids coding for enzymes or functionally active fragments thereof encoded by aminocoumarin biosynthetic gene clusters, to novel aminocoumarin compounds as well as to a method for the production of modified aminocoumarins utilizing the genetic information contained in said aminocoumarin biosynthetic gene clusters.

The aminocoumarin antibiotics novobiocin (marketed as Albamycin® by Pharmacia & Upjohn), clorobiocin, coumermycin A₁ and simocyclinone are produced by different strains of Streptomyces.

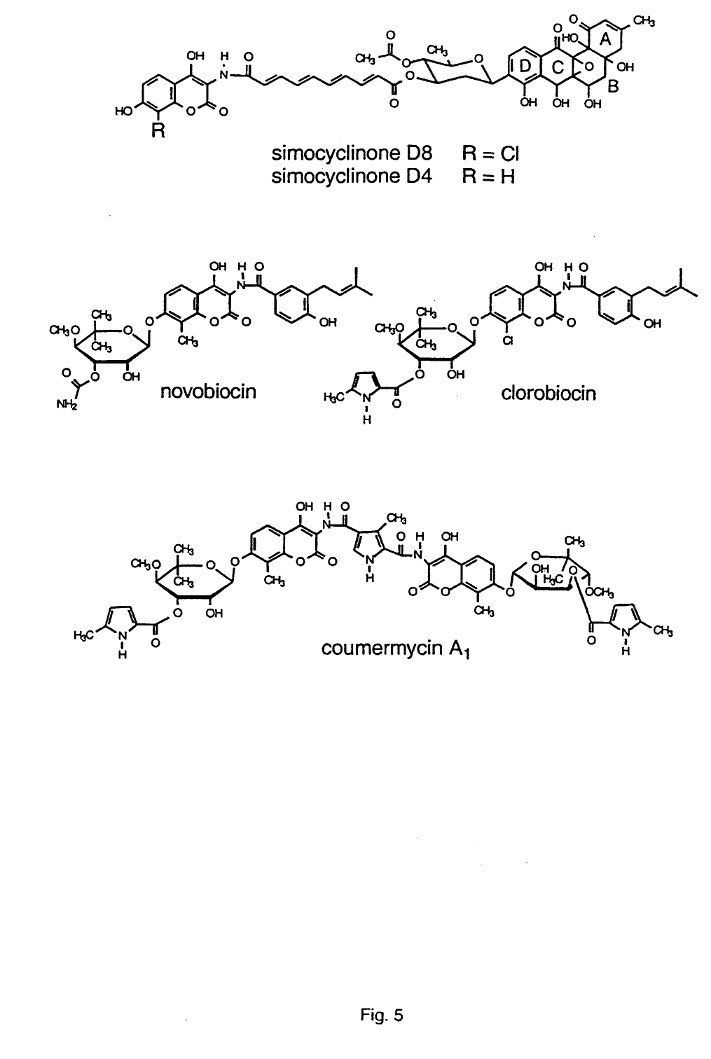

Bacterial DNA gyrase represents the major target of these coumarins (Maxwell (1997) Trends Microbiol. 5:102-109). X-ray crystallographic examinations demonstrated that the aminocoumarin moiety and the substituted deoxysugar moiety of these compounds (see FIG. 5) are essential for the binding to the gyrase B subunits of bacterial gyrase.

The affinity of these antibiotics to intact gyrase is extraordinarily high (Celia et al. (1994) J. Mol. Biol. 236: 618-628; Lewis et al. (1996) EMBO J. 15:141-1420; Tsai et al. (1997) Proteins: Struct. Funct. Genet. 28:41-52; Wigley et al. (1991) Nature 351:624-629).

Furthermore, the coumermycin A₁ molecule (FIG. 5) contains two of these active aminocoumarin-deoxysugar moieties and has been shown to stabilize a dimeric form of the 43-kDa GyrB fragments (Ali et al. (1993) Biochemistry 32: 2717-2724; Gormley et al. (1996) Biochemistry 35: 5083-5092). Therefore, coumermycin A₁ is likely to crosslink the two gyrase B subunits of the intact gyrase heterotetramer which consists of two gyrase A to gyrase B subunits. Consequently, the affinity of this antibiotic to intact gyrase is extremely high: 50% inhibition of gyrase is achieved by coumermycin A₁ at a concentration of only 0.004 μM (Peng et al. (1993) J. Biol. Chem. 268: 24481-24490).

The extremely high affinity of aminocoumarins to intact gyrase makes these molecules very interesting starting compounds for the development of novel antibiotics which may serve as antiinfectives against multi-resistant gram-positive bacteria, as remedies against malignant diseases (Rappa et al. (1992) Int. J. Cancer 51: 780-787; Lorico et al. (1992) Int. J. Cancer 52: 903-909) or for other purposes.

Recently, the novobiocin biosynthetic gene cluster of Streptomyces spheroides has been cloned and sequenced (Steffensky et al. (2000) Antimicrobiol. Agents Chemother. 44: 1214-1222).

The technical problem underlying the present invention is therefore to provide further nucleic acids coding for enzymes involved in the biosynthesis of aminocoumarins and to provide a novel system for the preparation of modified aminocoumarins based on this genetic information.

The solution to the above technical problem is achieved by the embodiments characterized in the claims.

In particular, the present invention relates to an isolated nucleic acid having a nucleotide sequence coding for at least one enzyme or a functionally active fragment thereof encoded by an aminocoumarin biosynthetic gene cluster, wherein said aminocoumarin is selected from the group consisting of coumermycin A₁, clorobiocin and simocyclinone.

According to a further embodiment, there is provided a method for the preparation of modified aminocoumarins based on the genetic information contained in the biosynthetic gene clusters of novobiocin, coumermycin A₁, clorobiocin and simocyclinone.

A further embodiment of the present invention relates to an aminocoumarin compound substantially being composed of structural elements derived from different aminocoumarins selected from the group consisting of novobiocin, clorobiocin, coumermycin A₁ and simocyclinone.

The “enzyme or functionally active fragment thereof” encoded by an aminocoumarin biosynthetic gene cluster characterizes at least one reaction in the biosynthesis of said aminocoumarin.

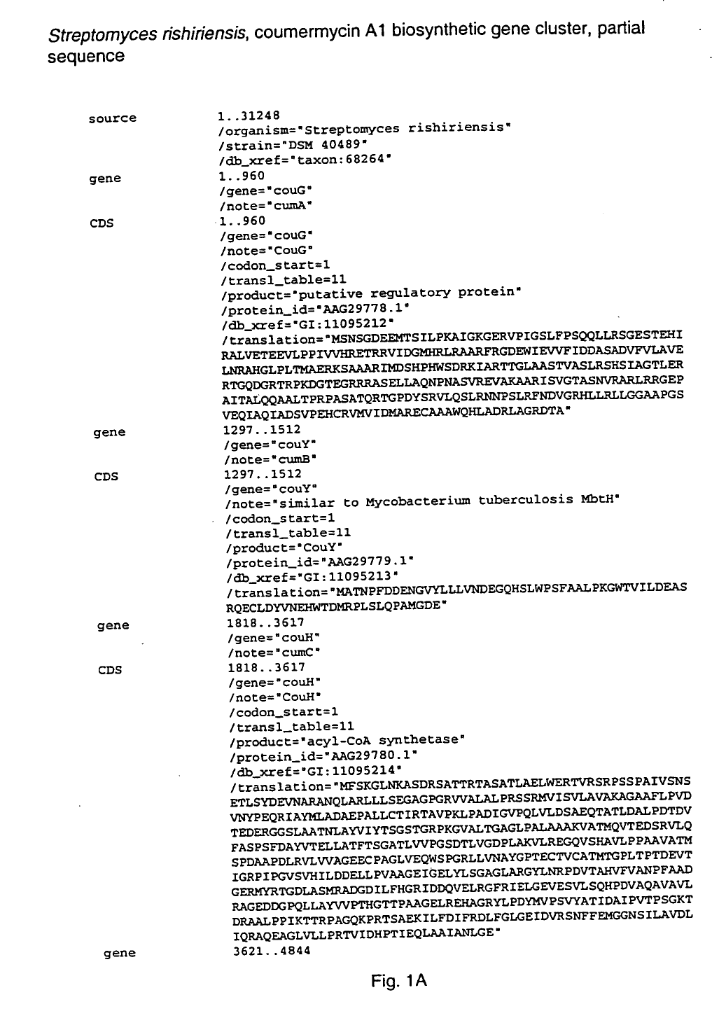

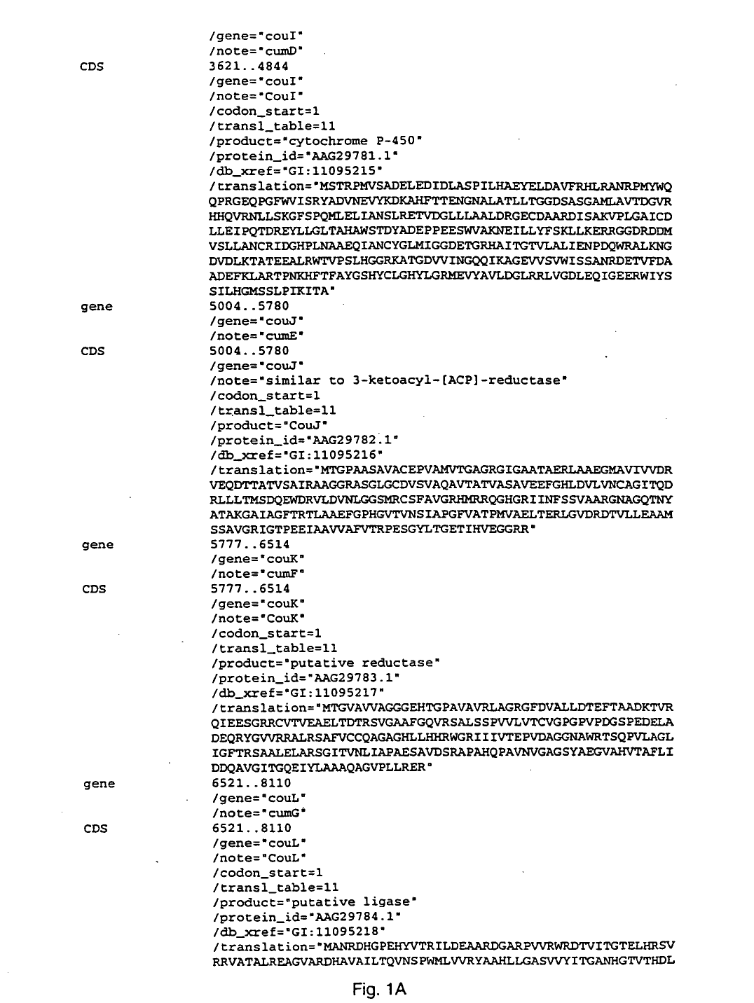

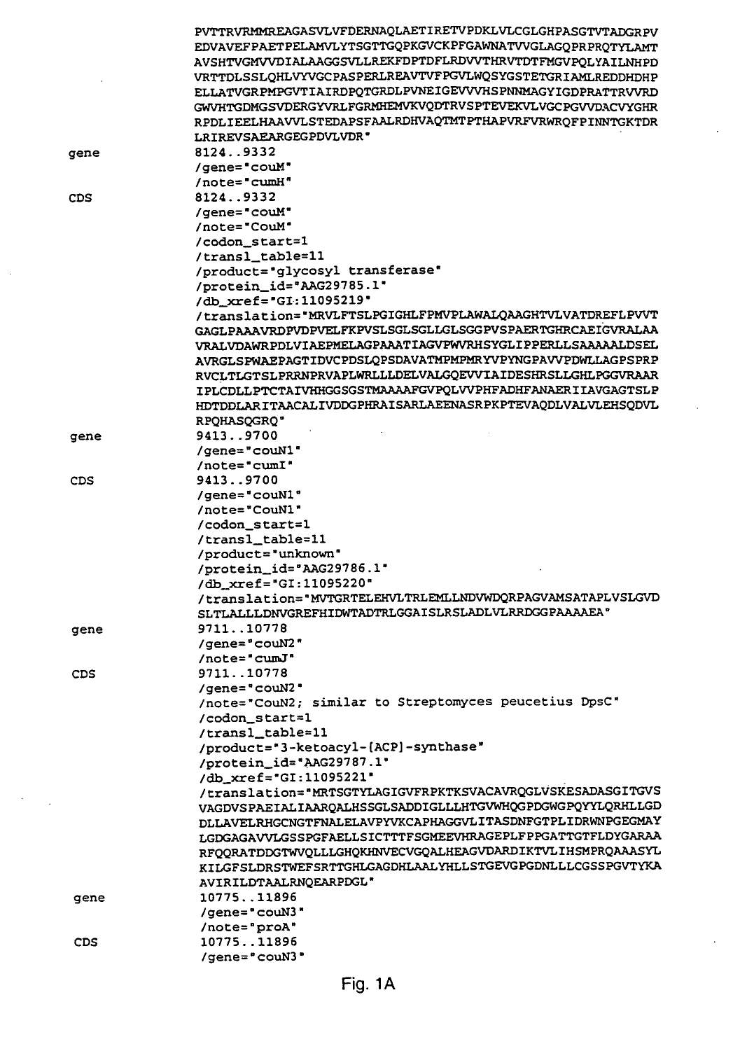

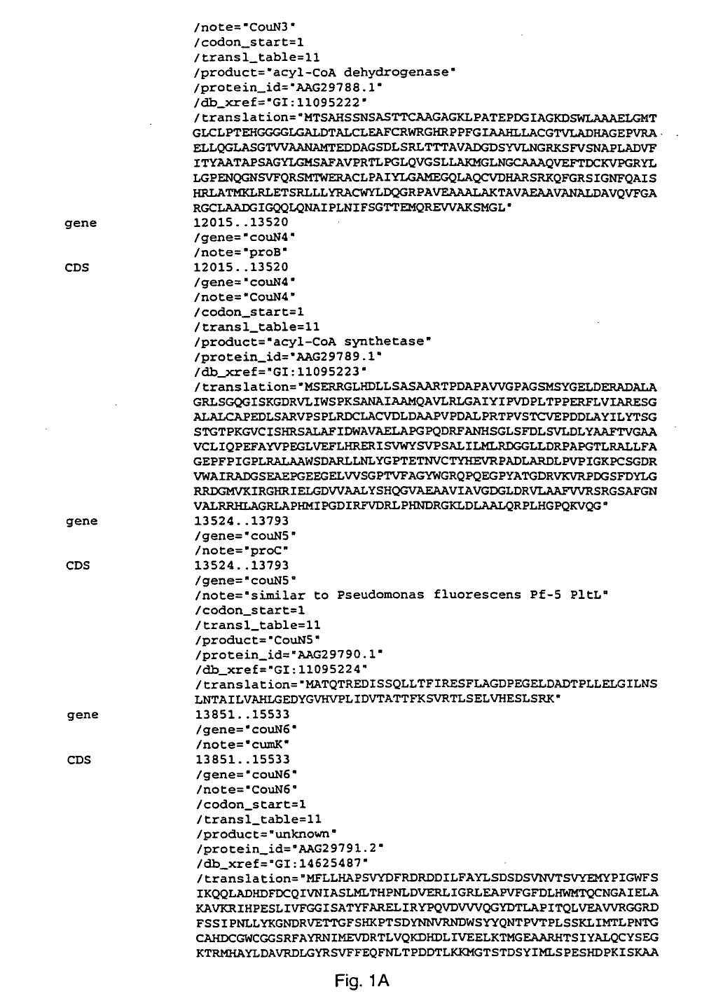

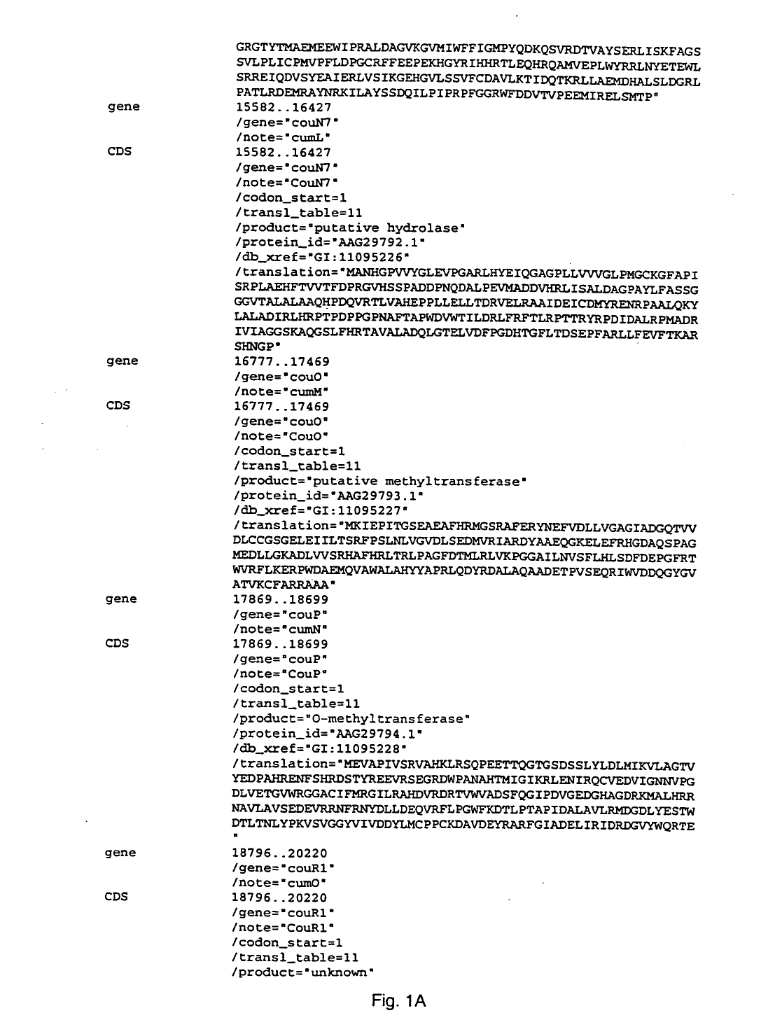

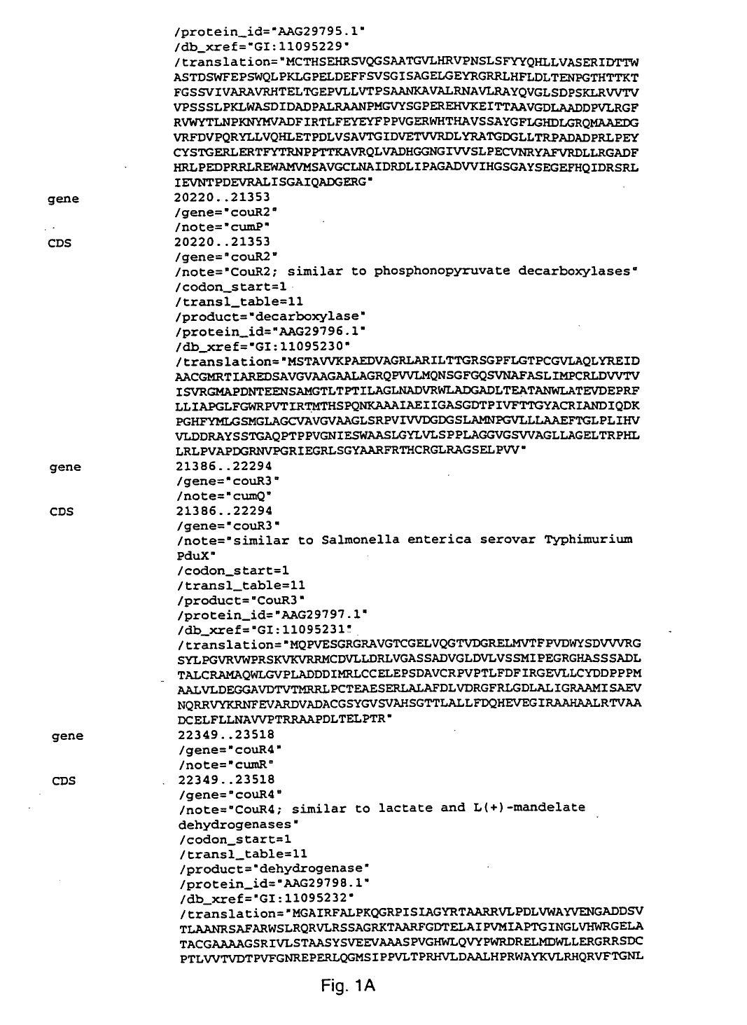

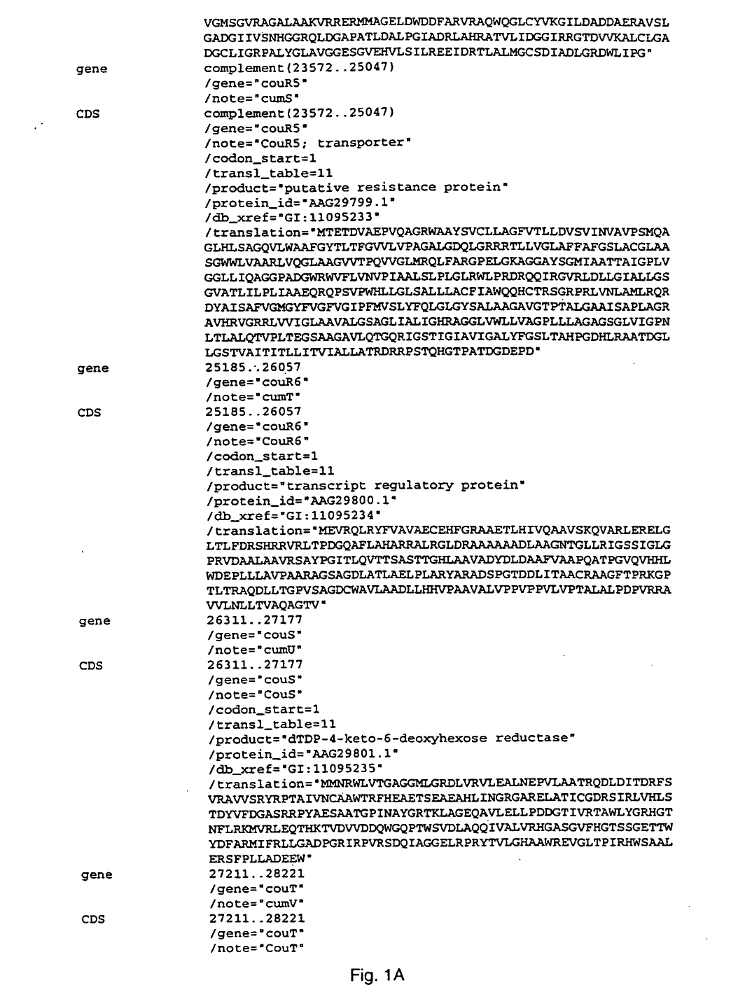

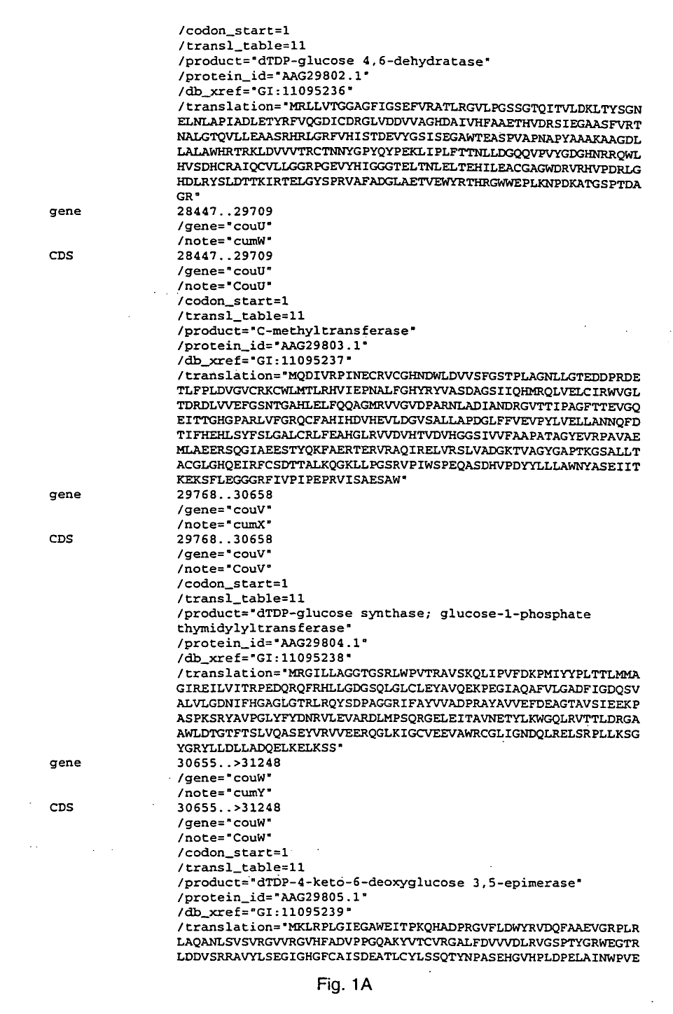

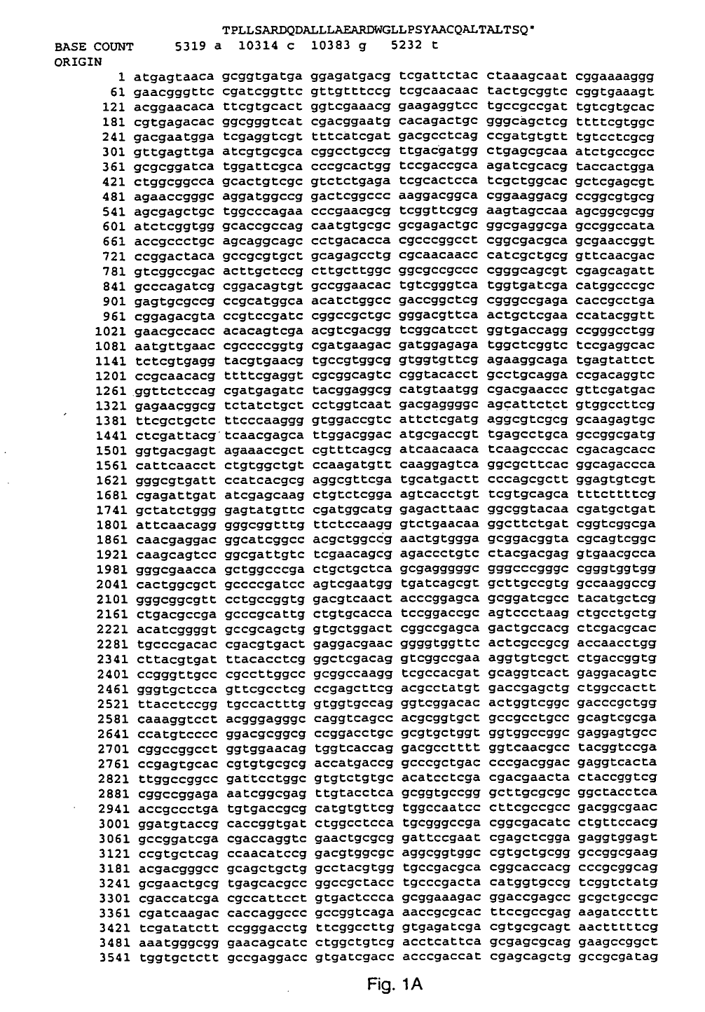

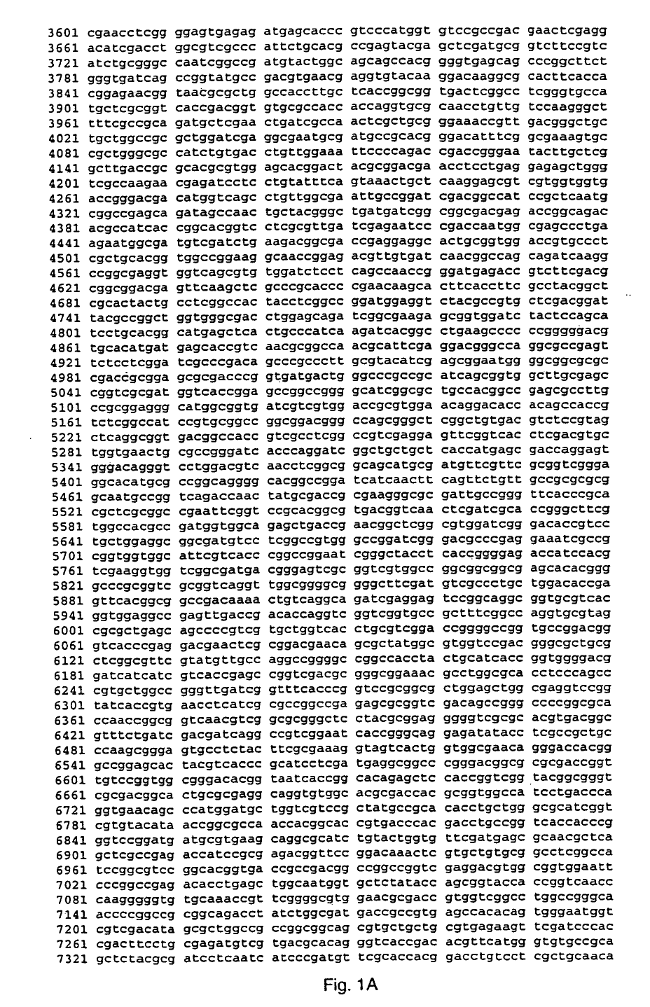

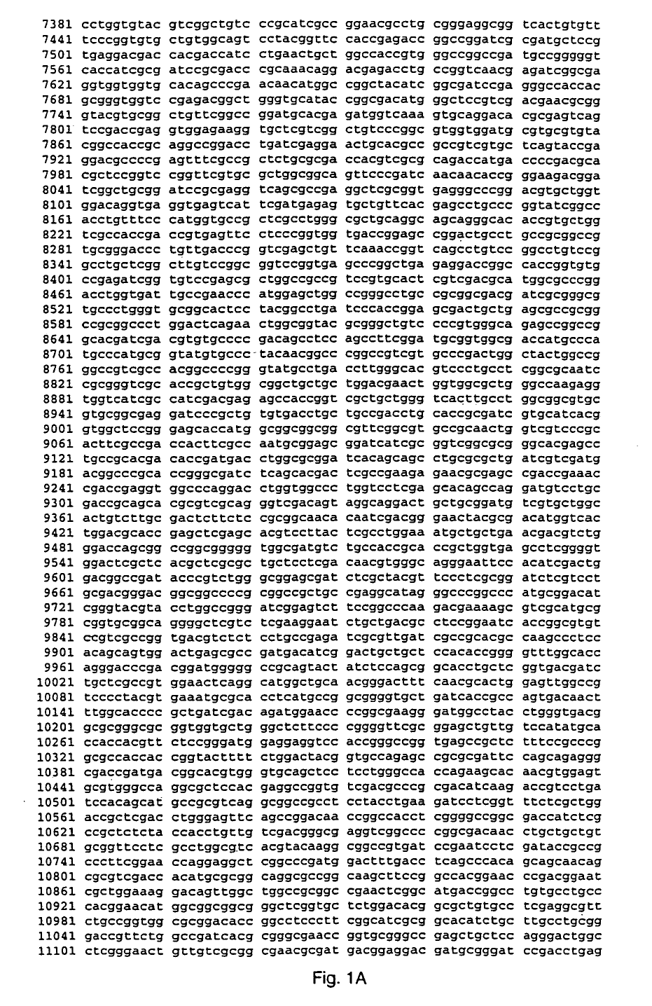

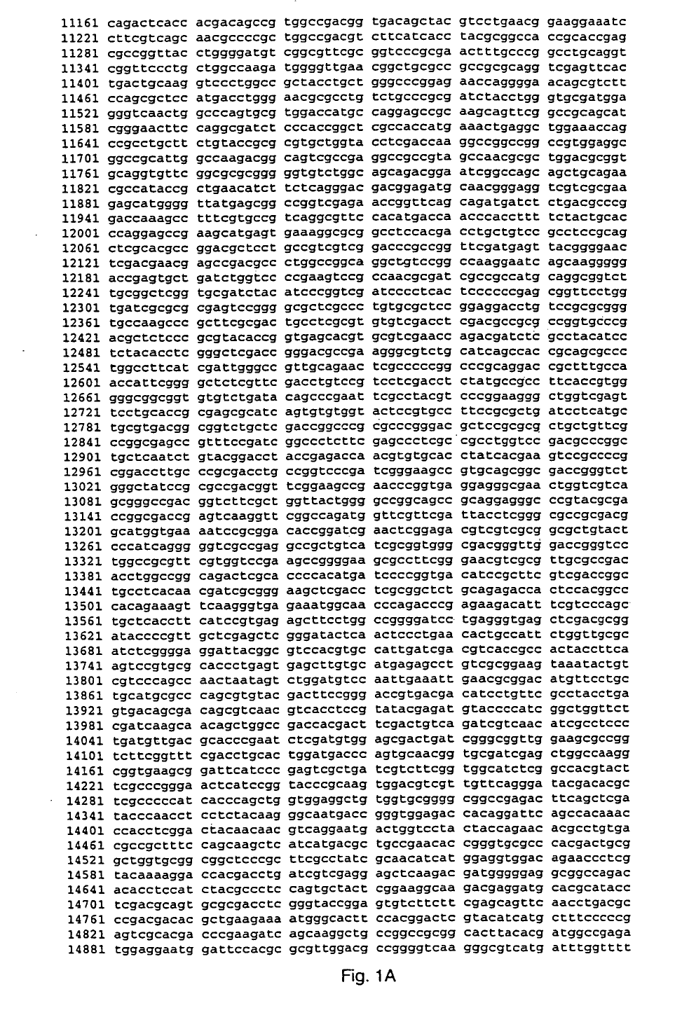

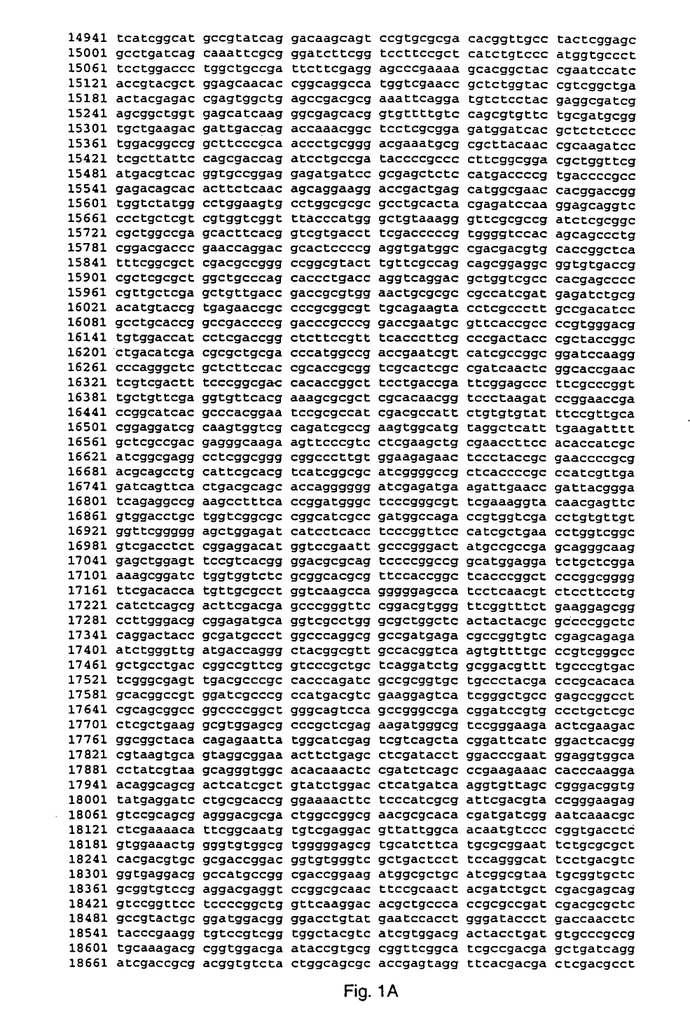

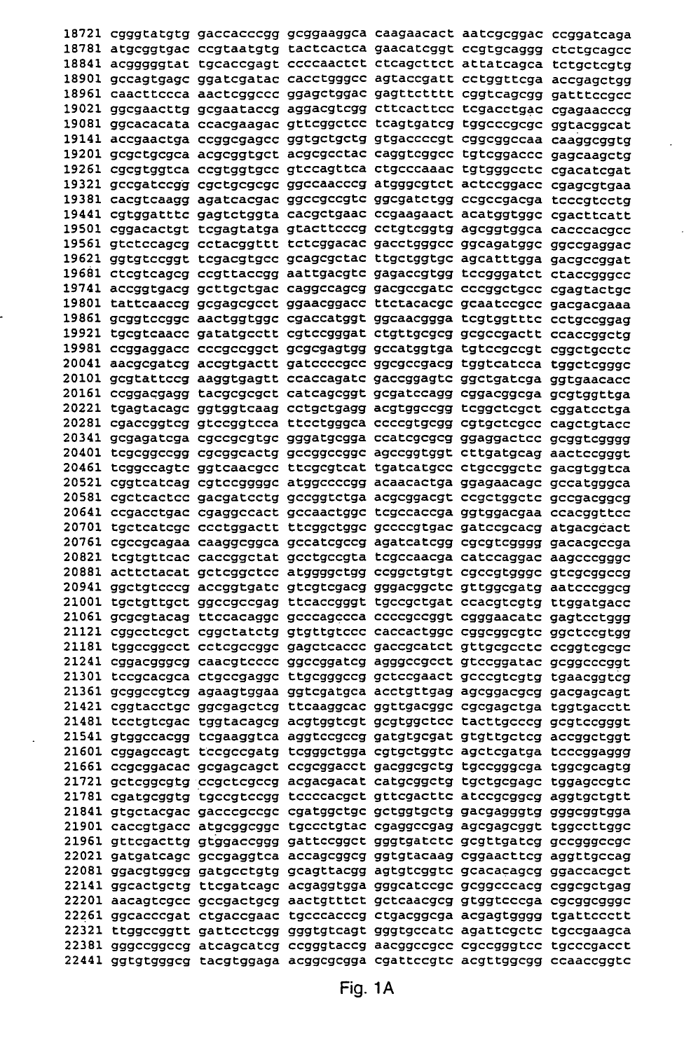

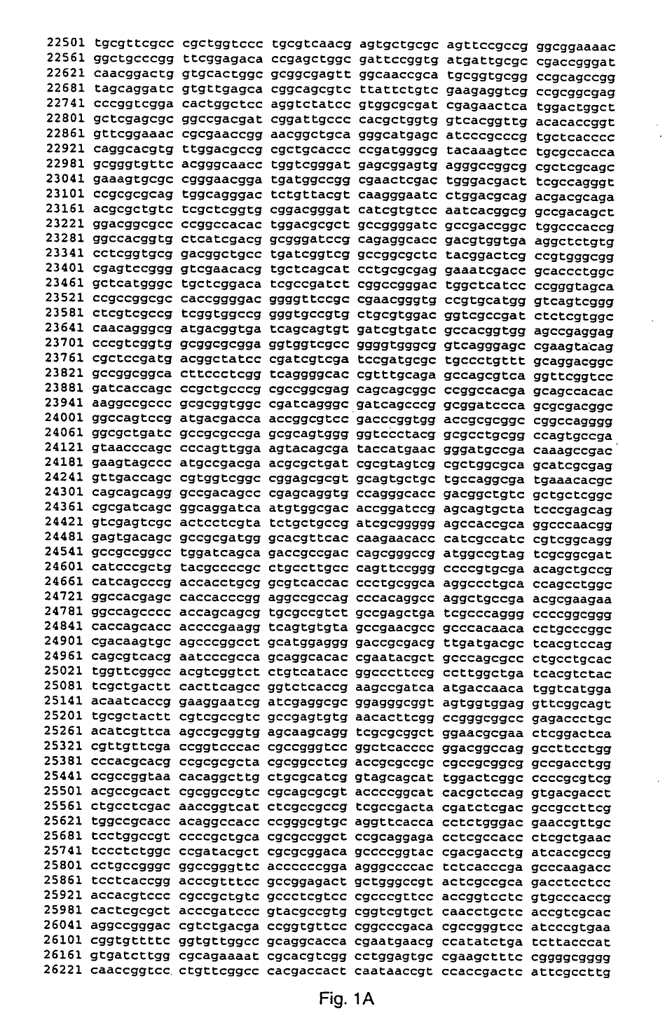

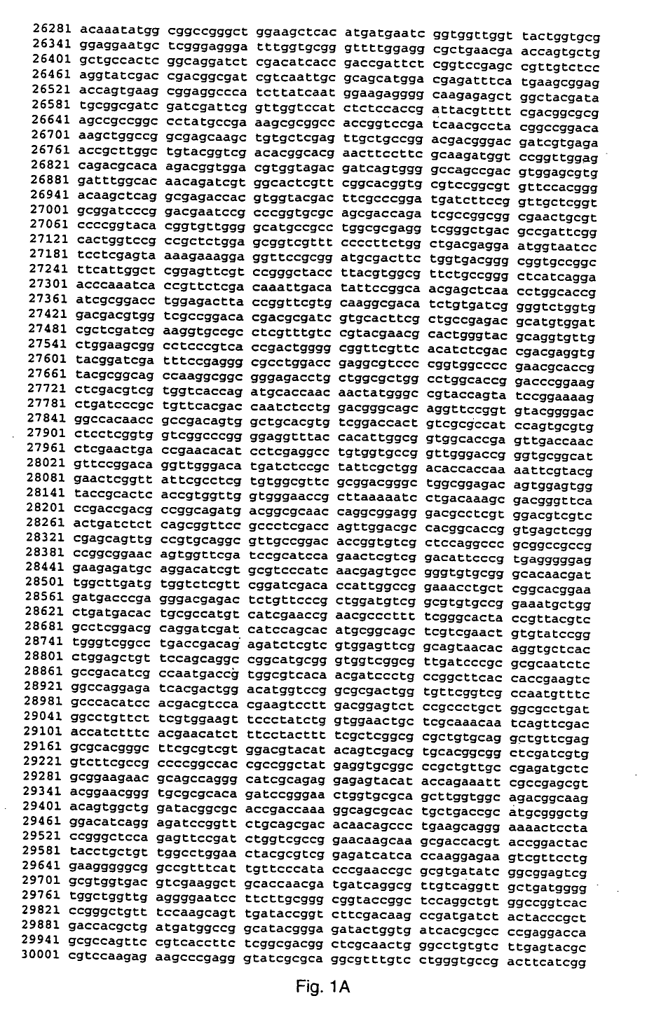

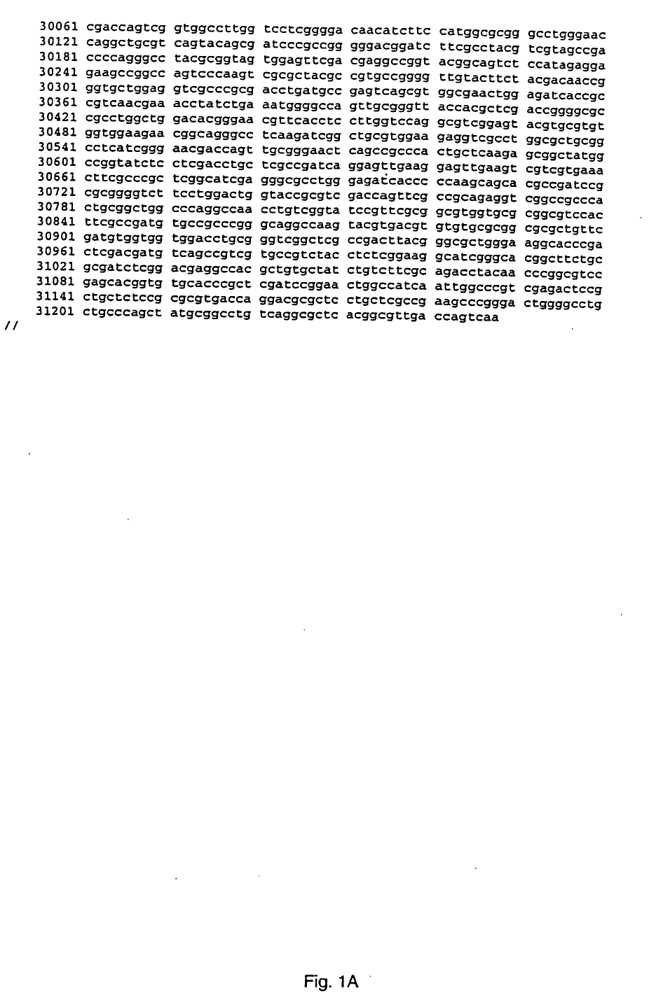

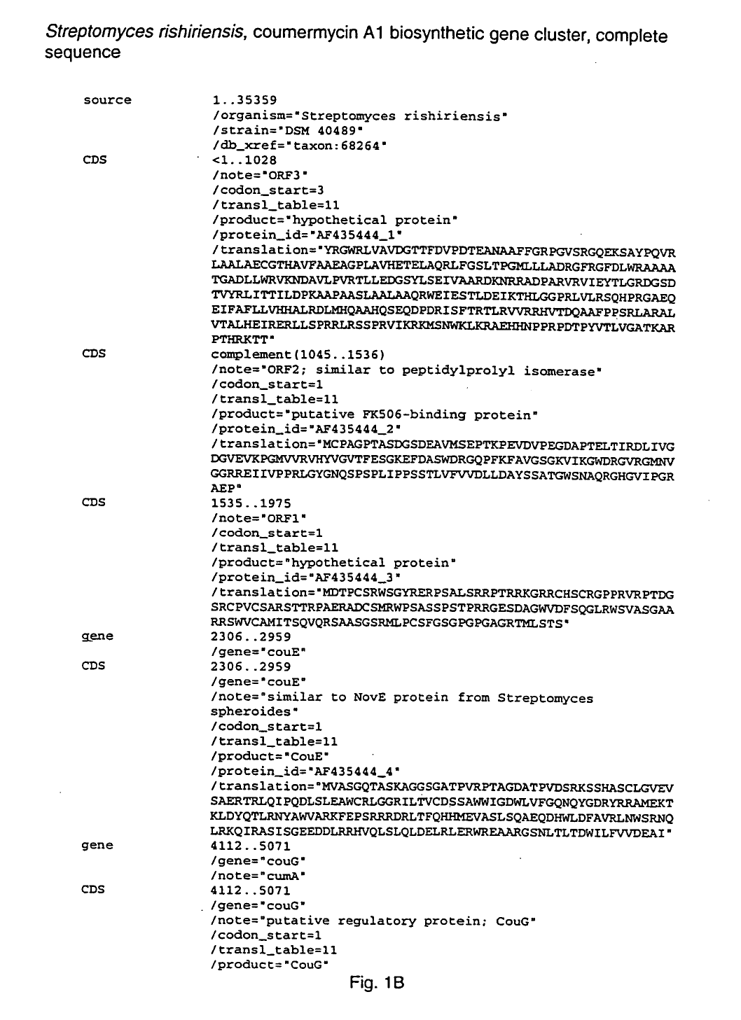

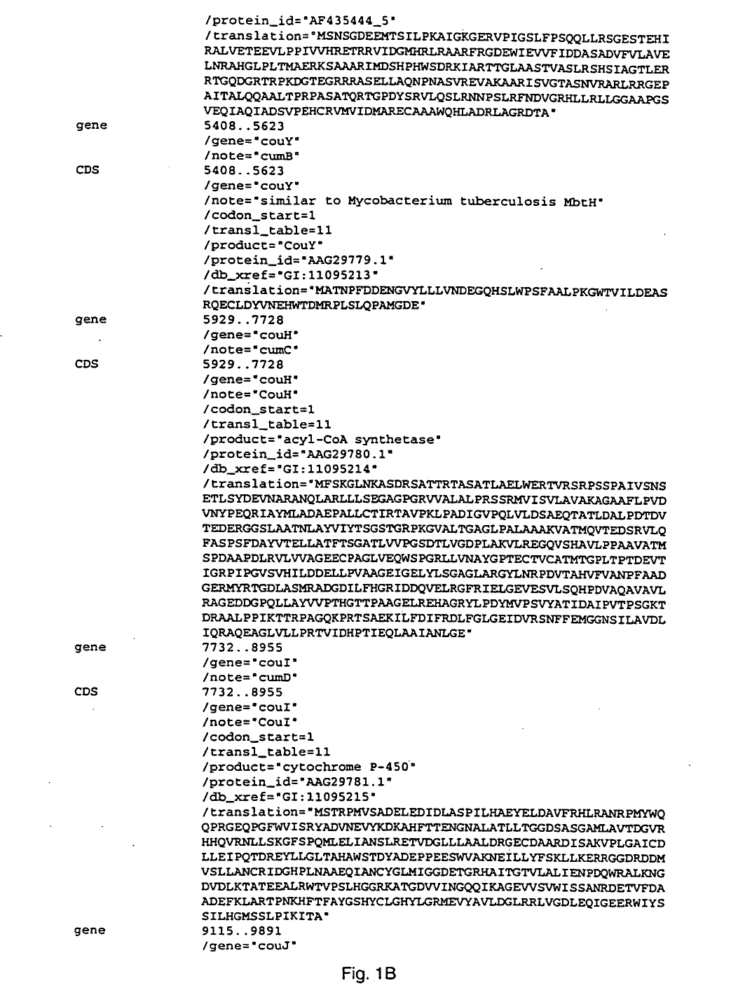

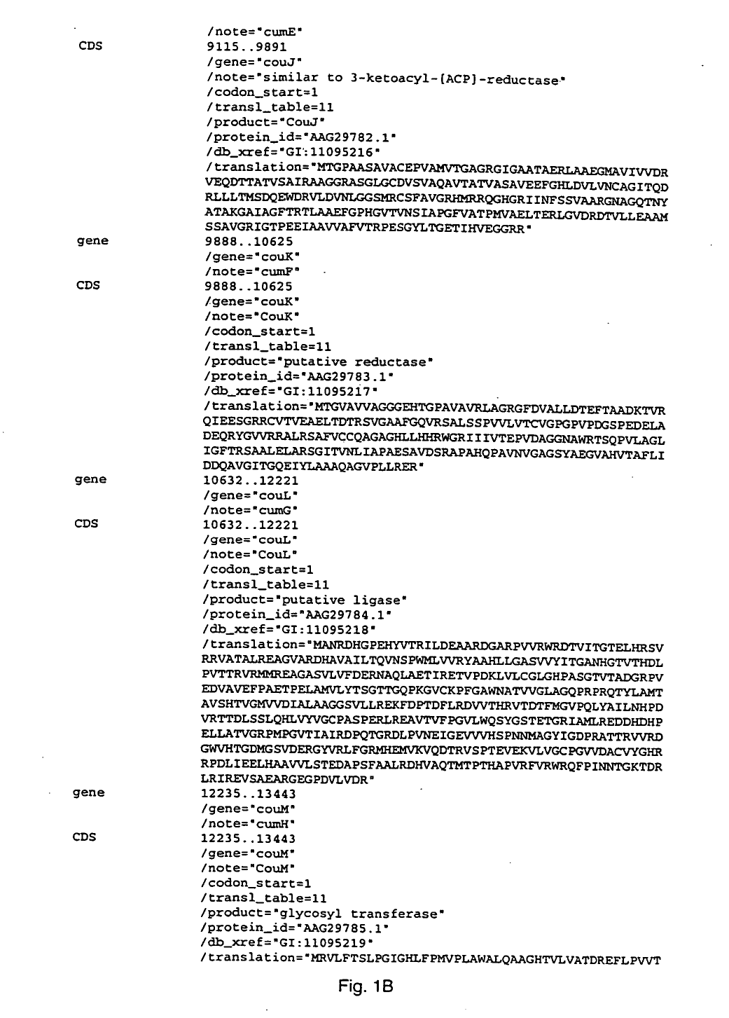

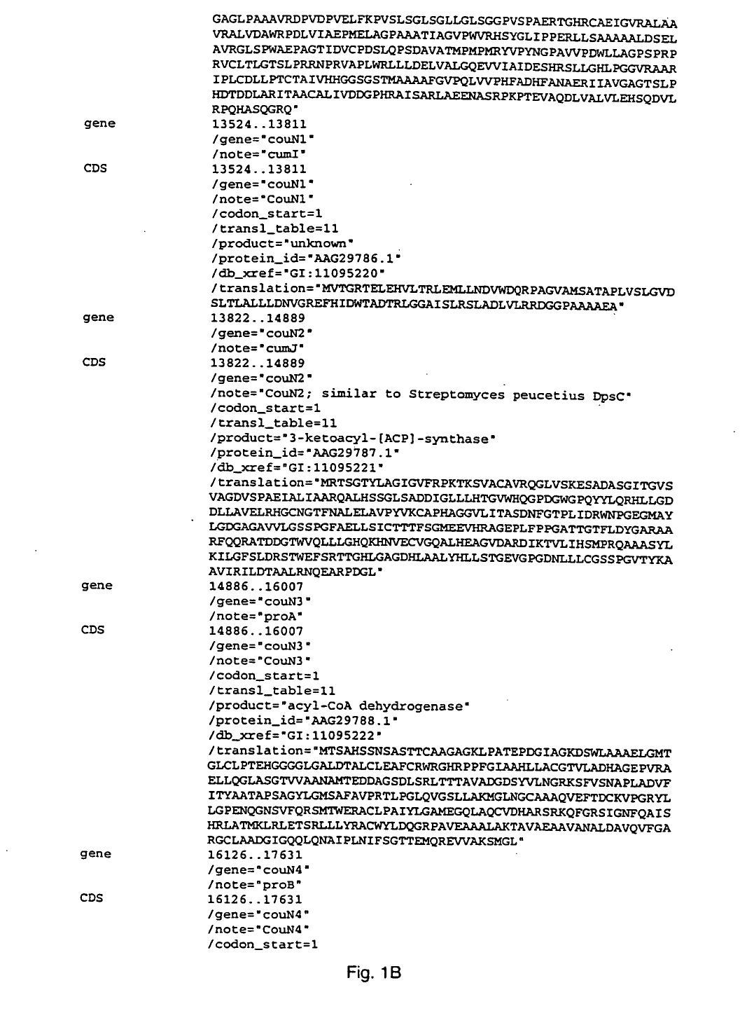

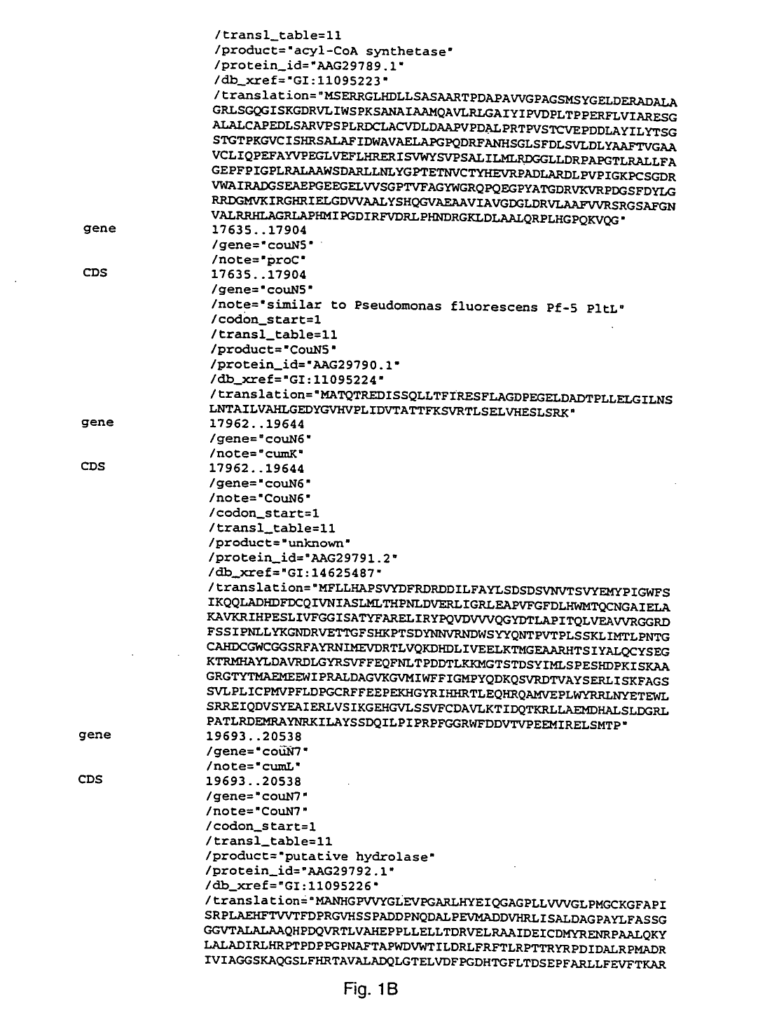

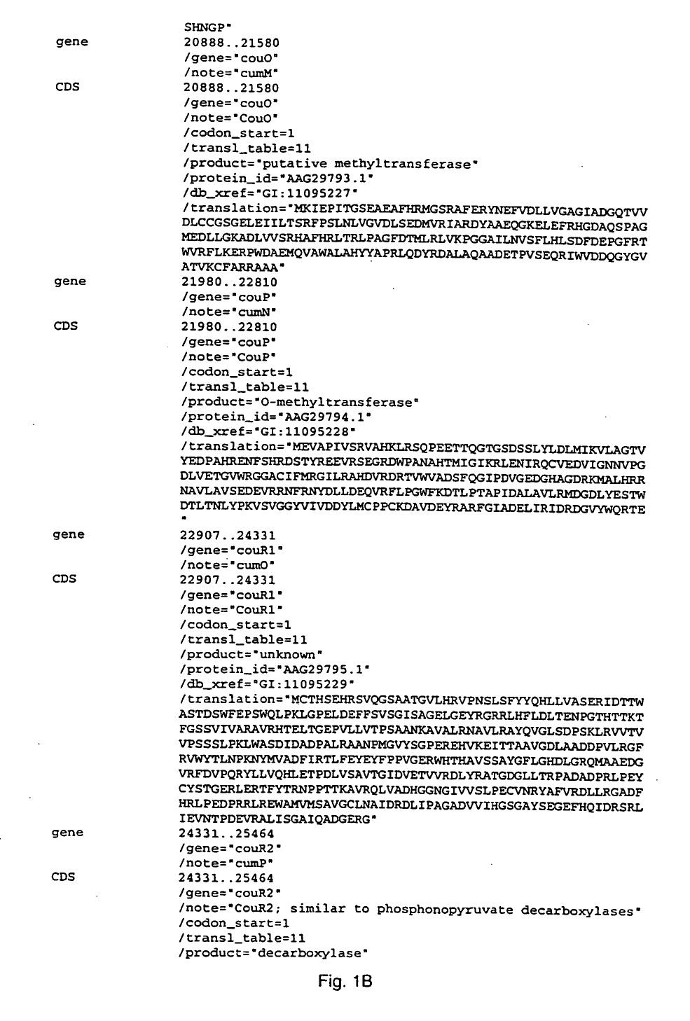

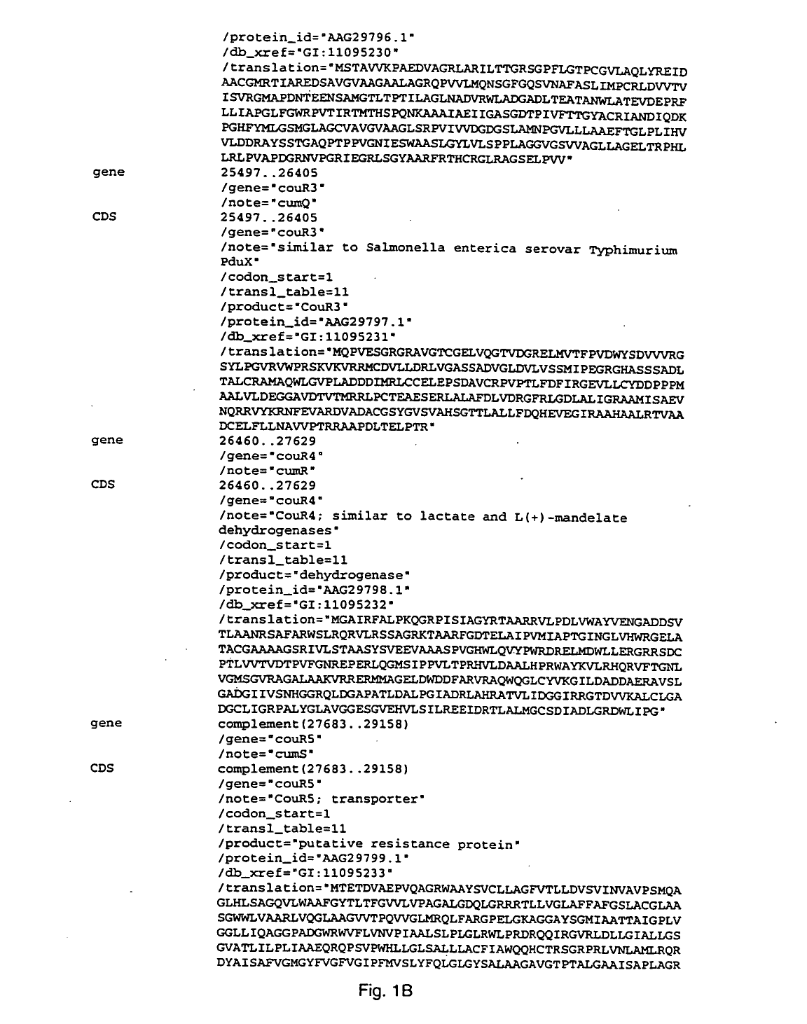

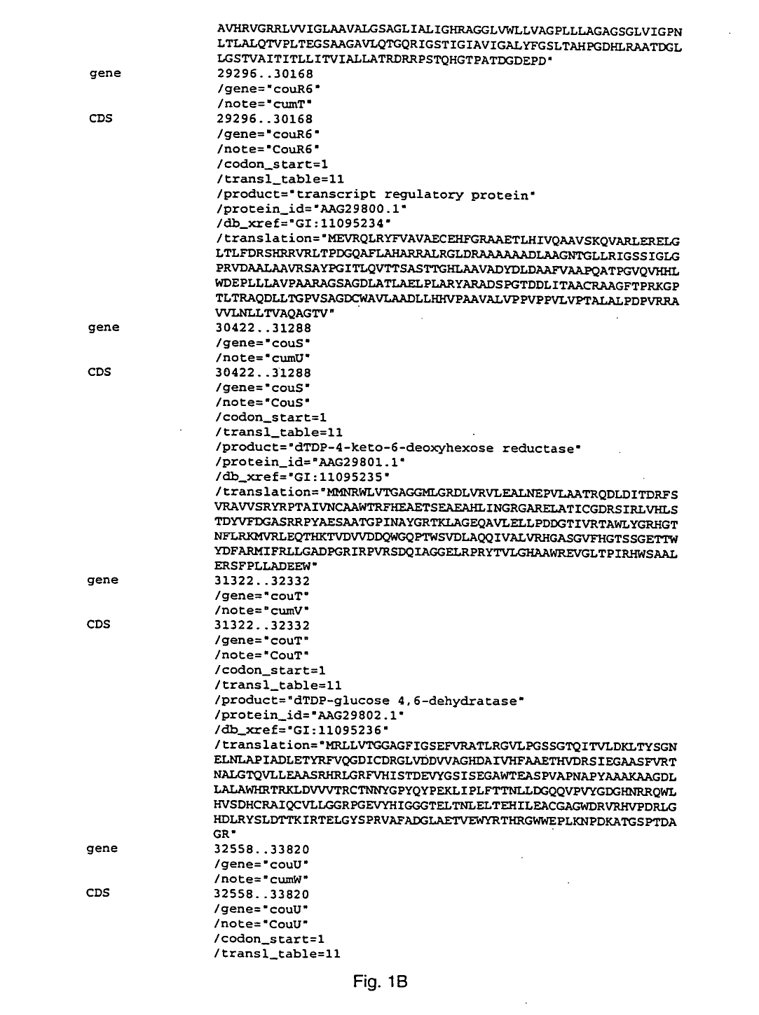

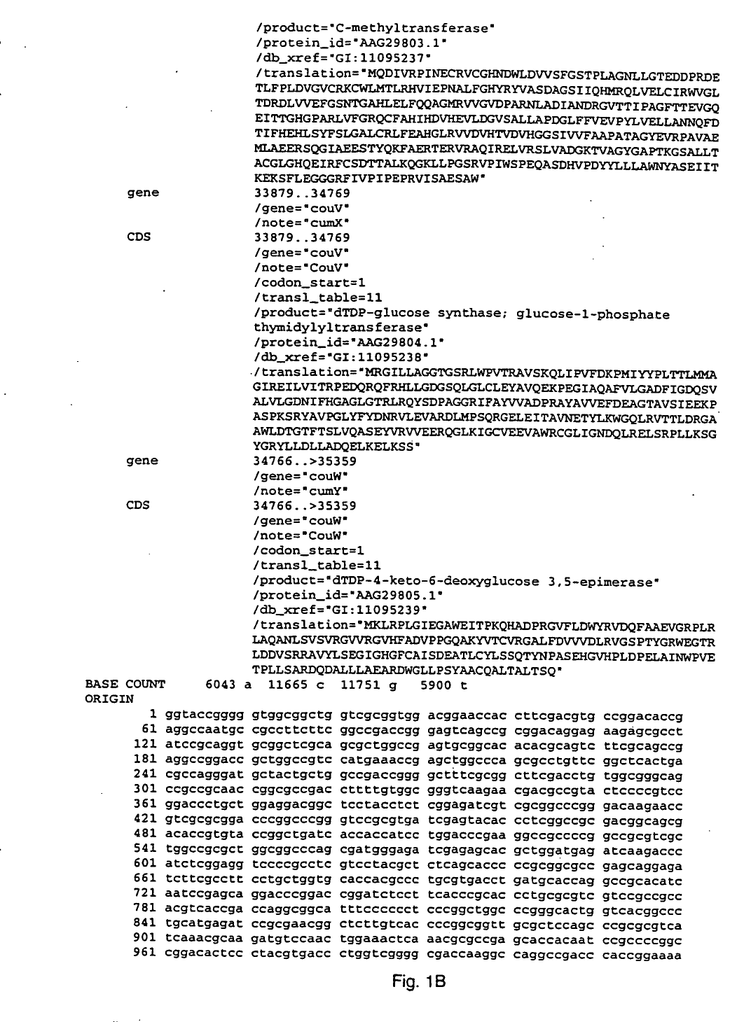

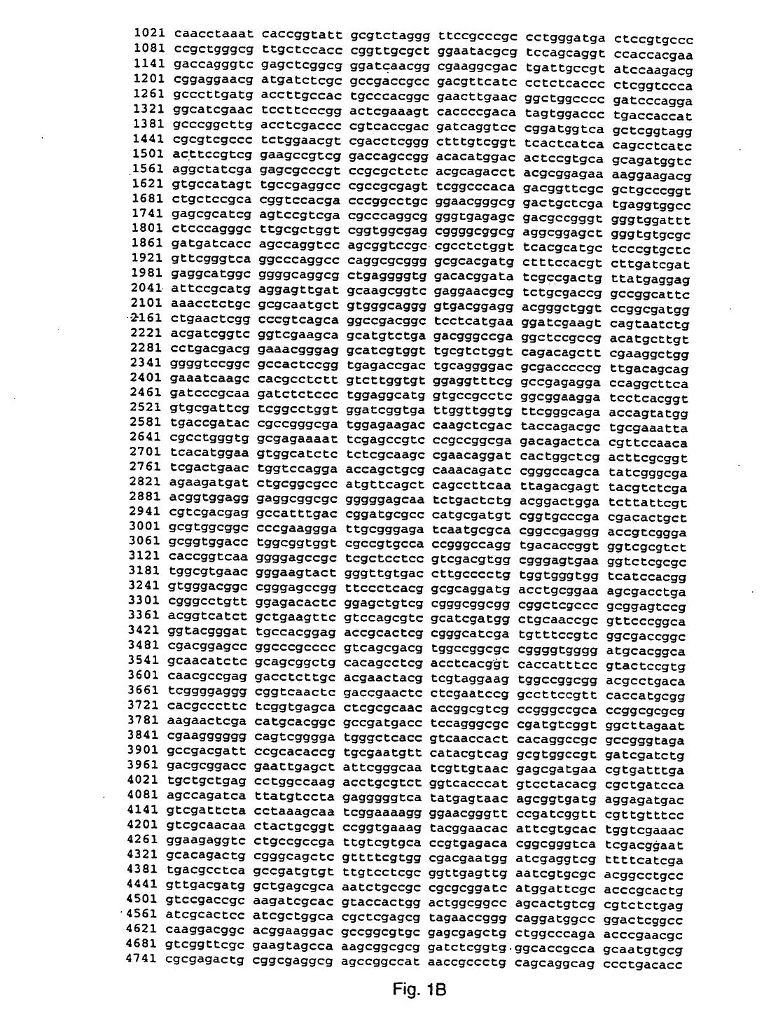

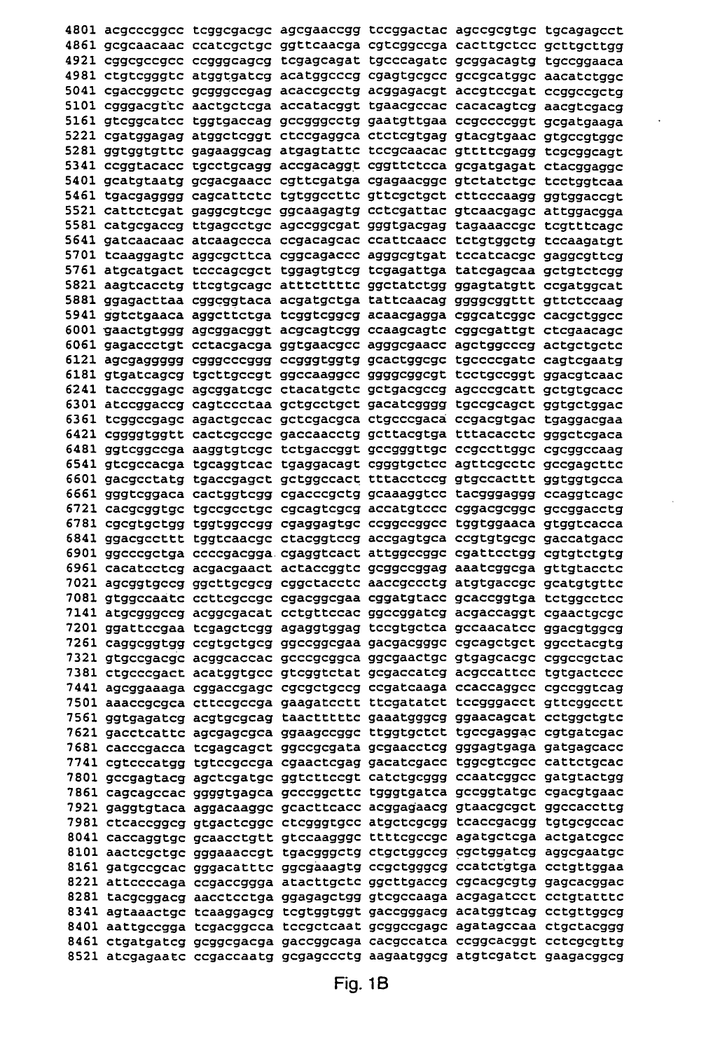

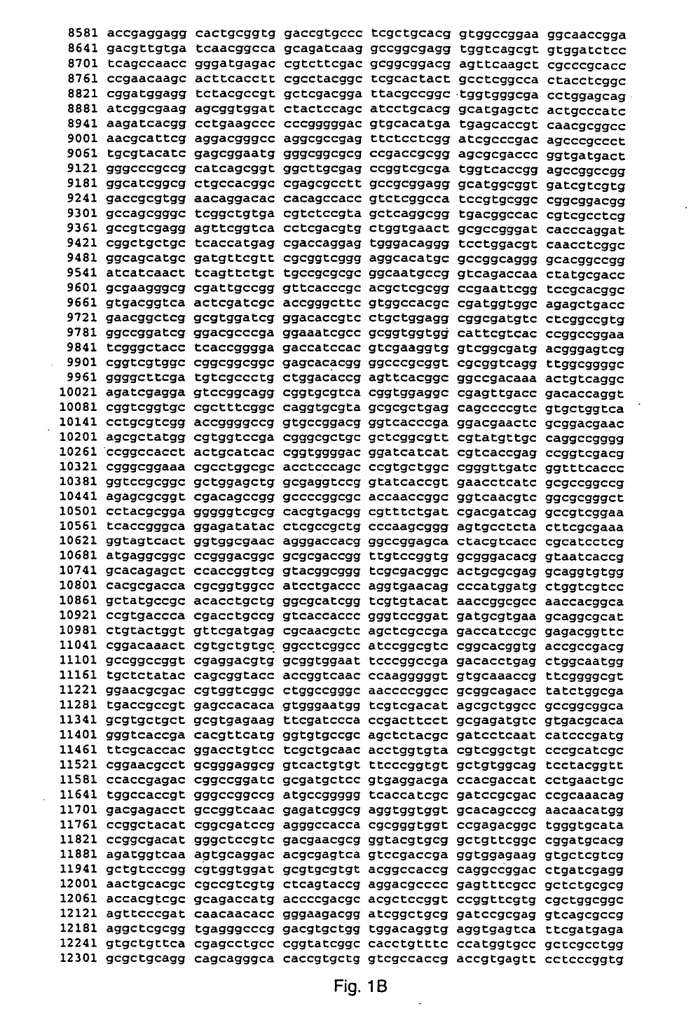

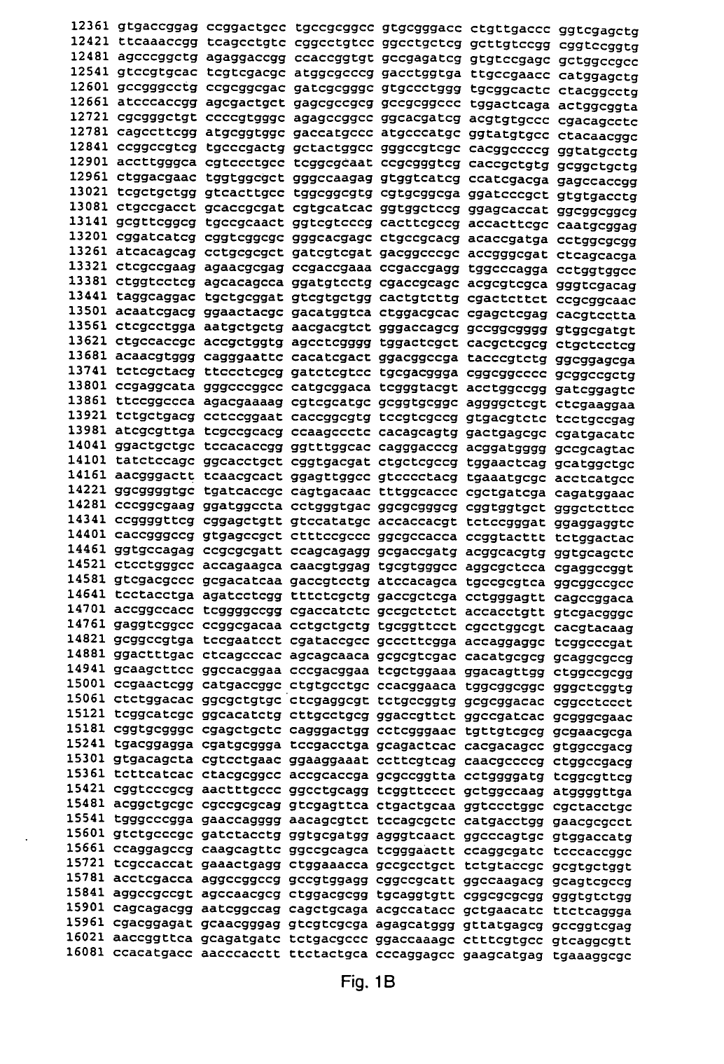

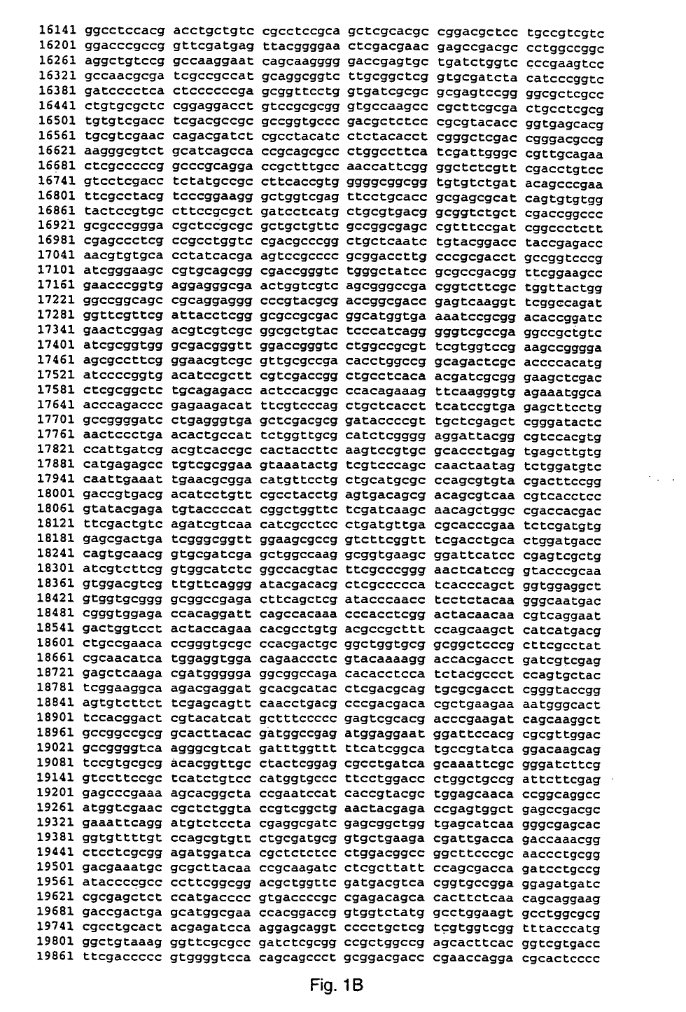

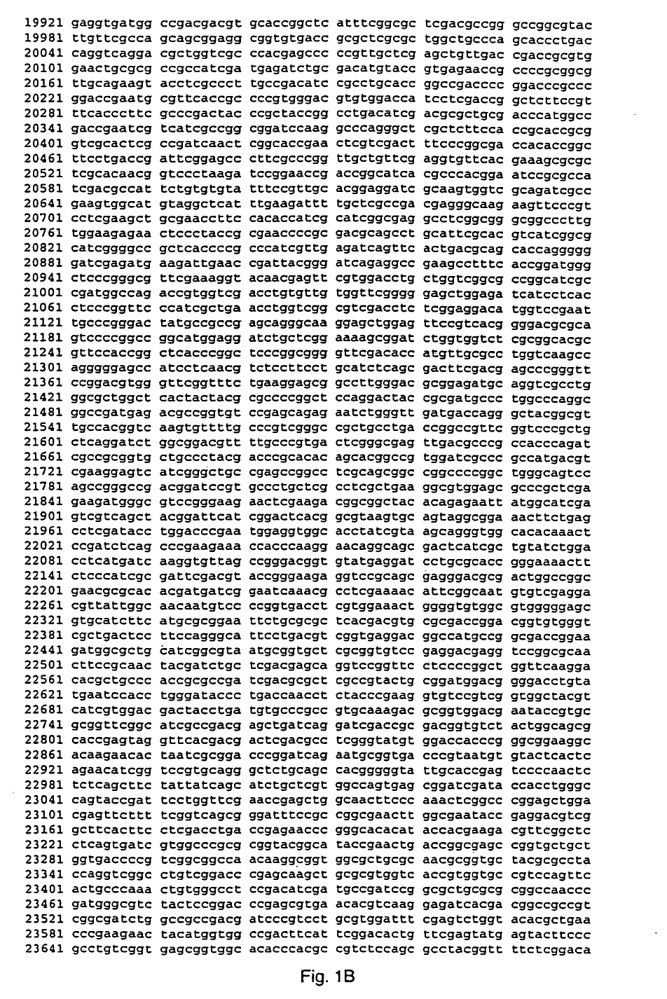

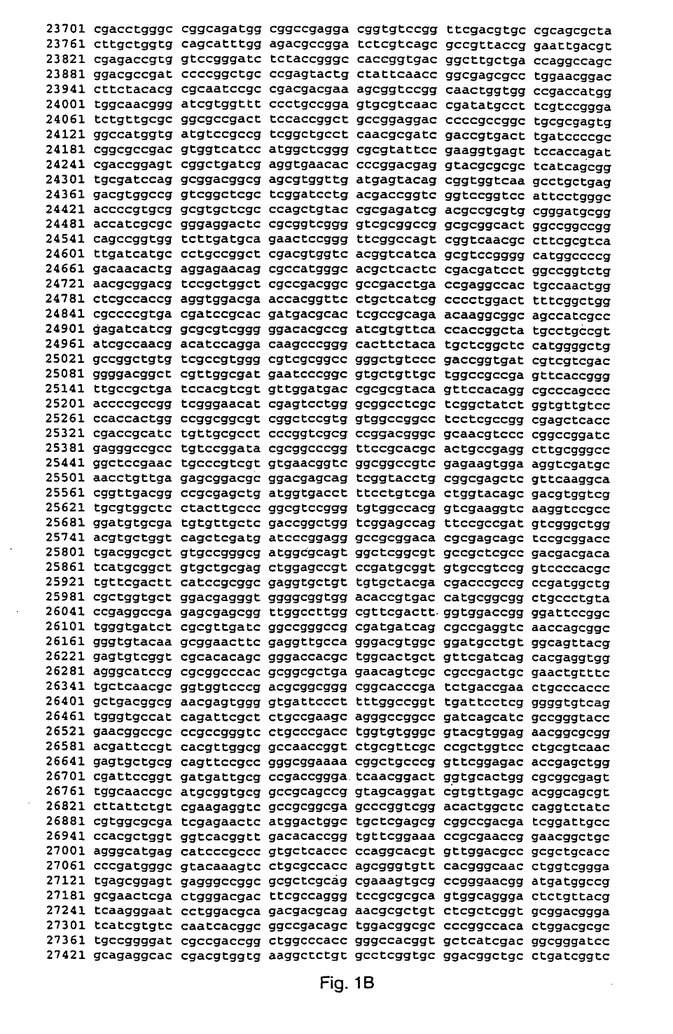

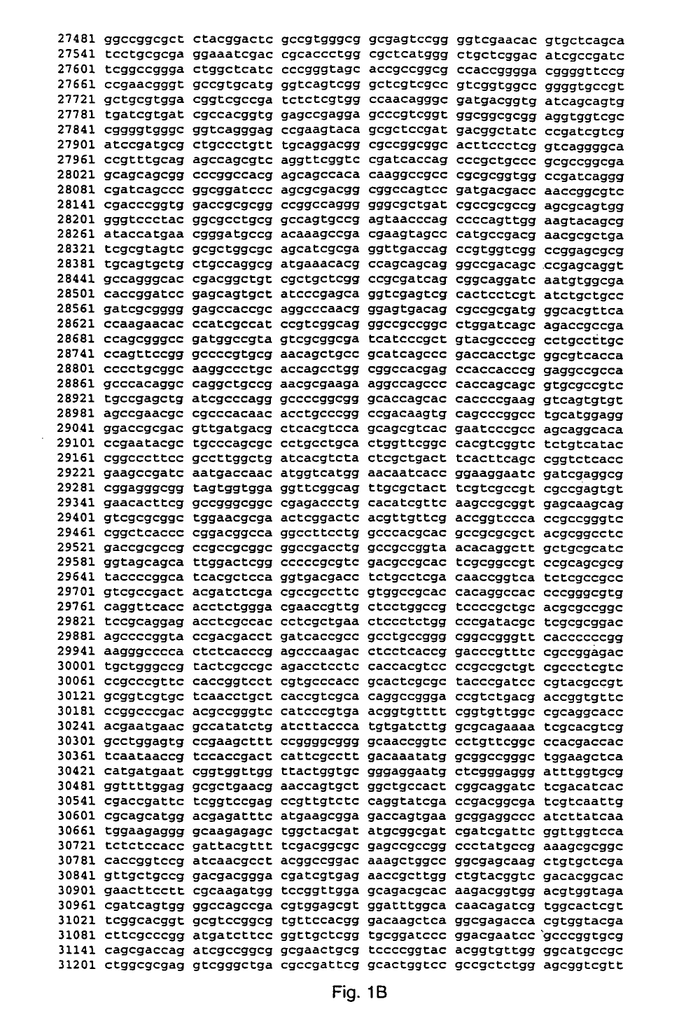

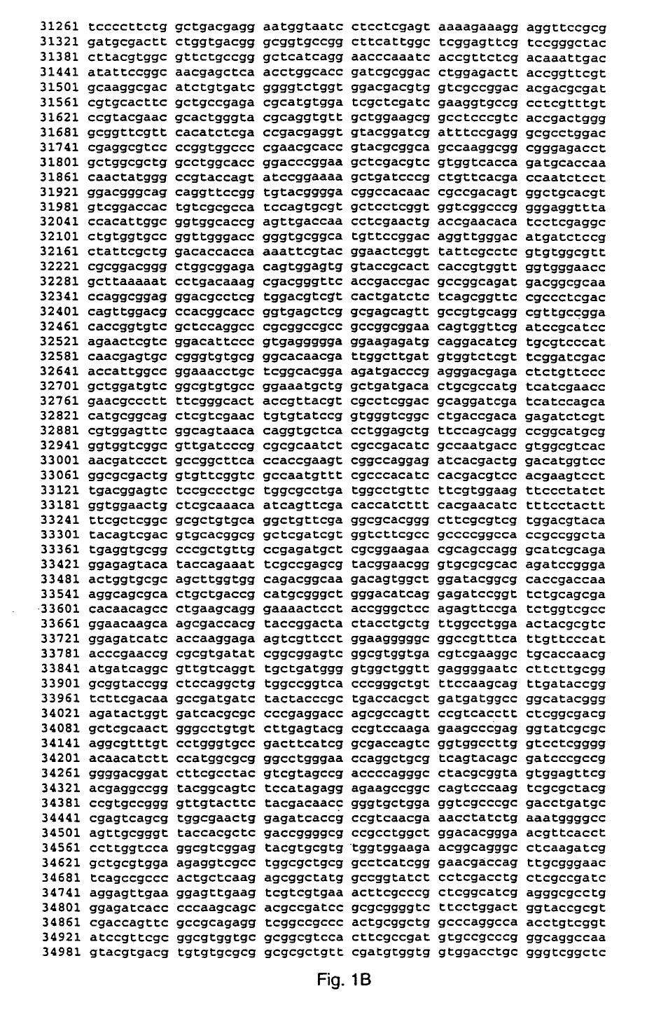

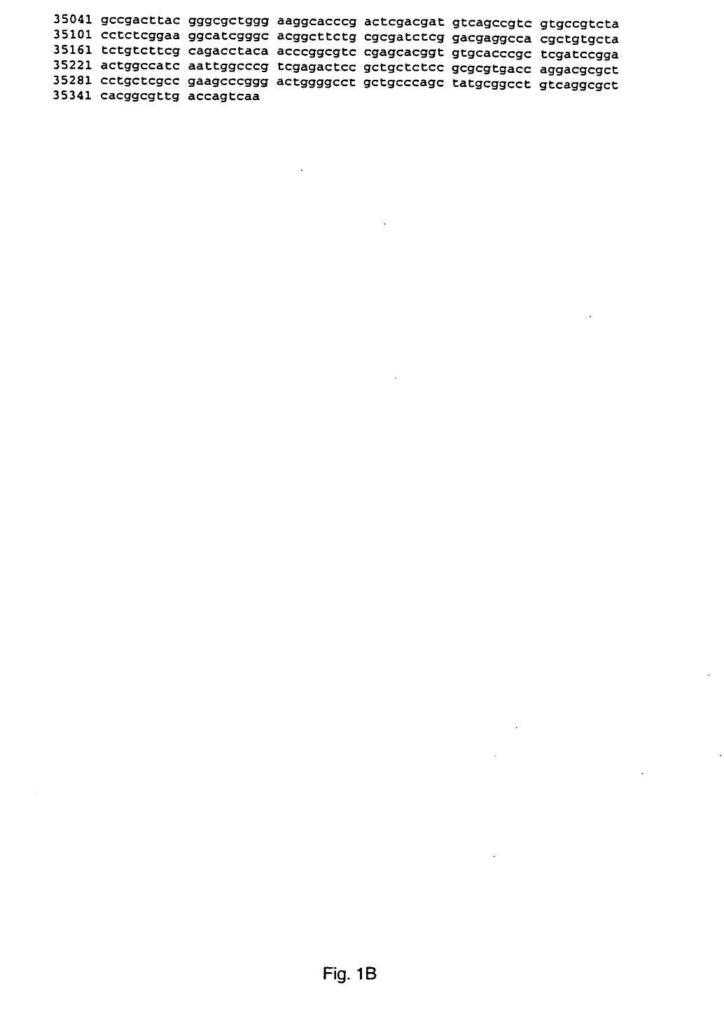

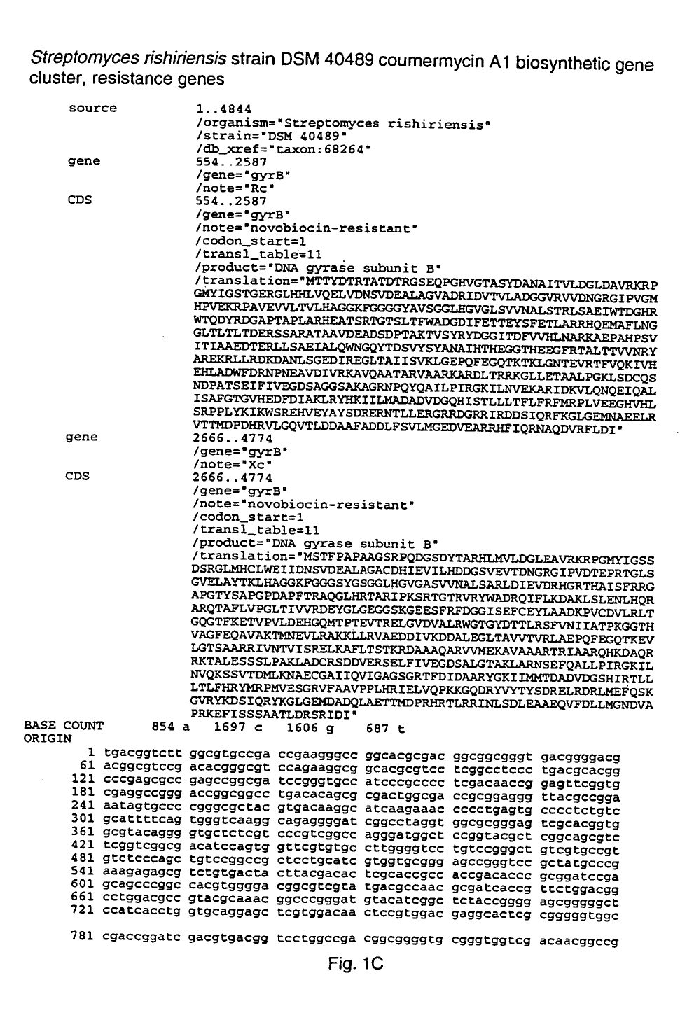

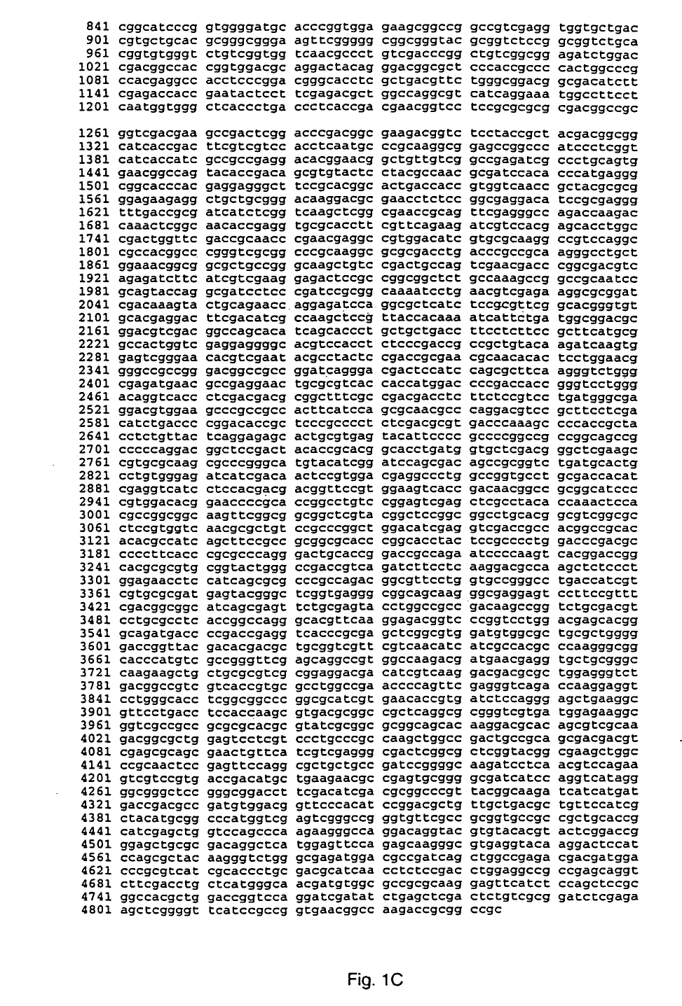

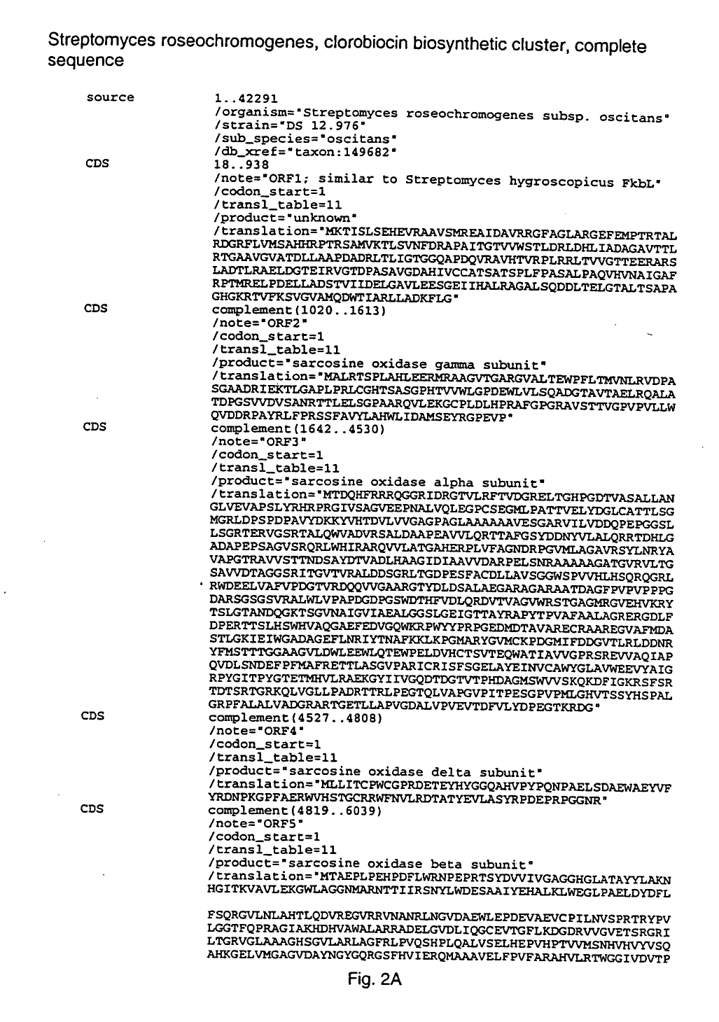

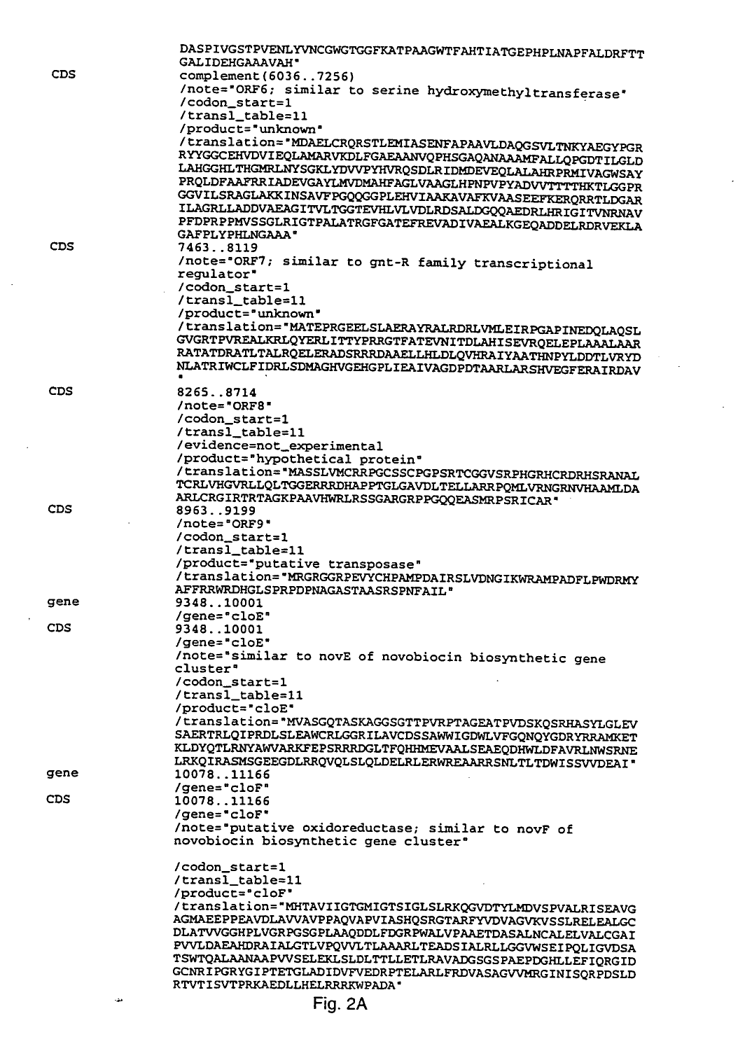

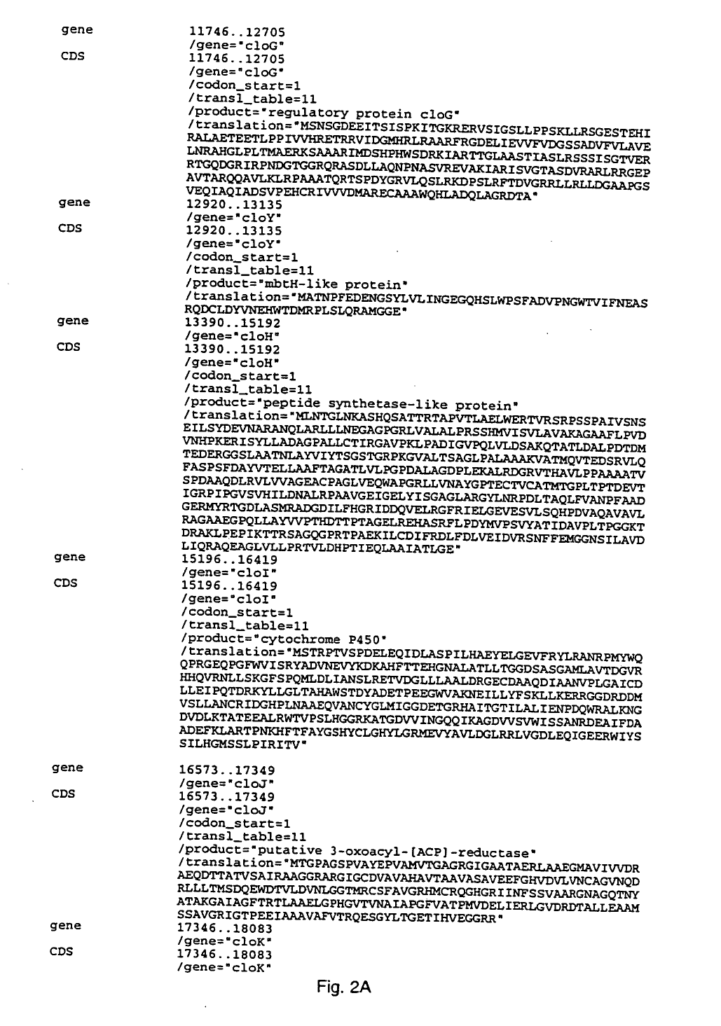

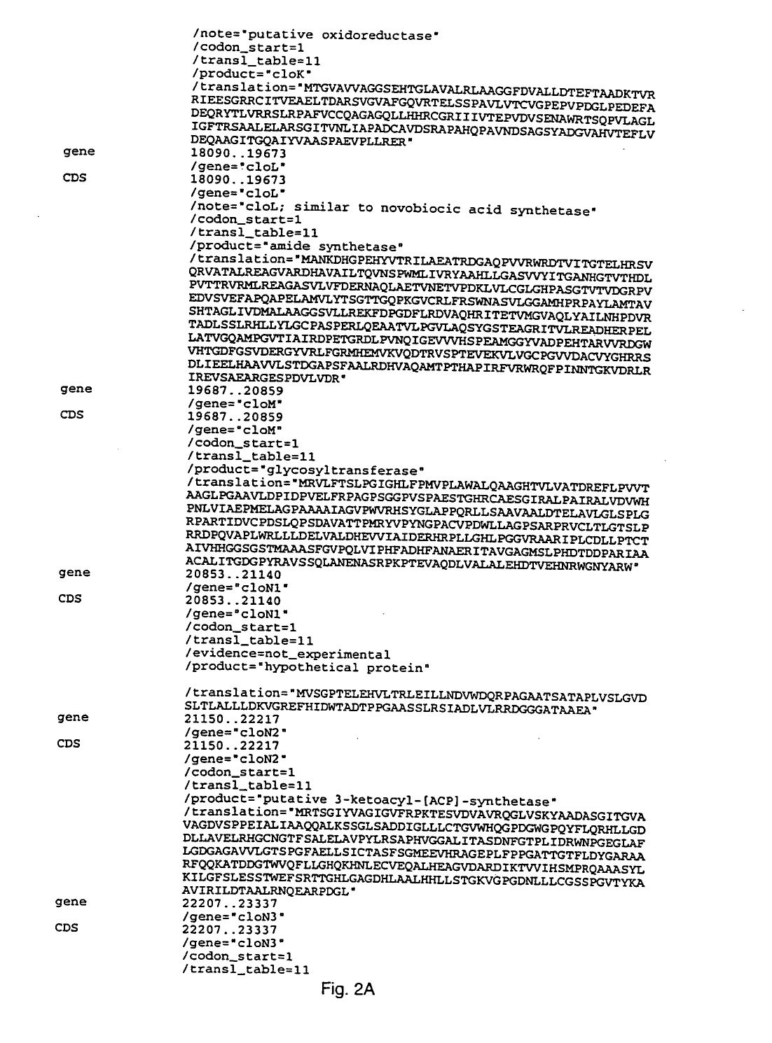

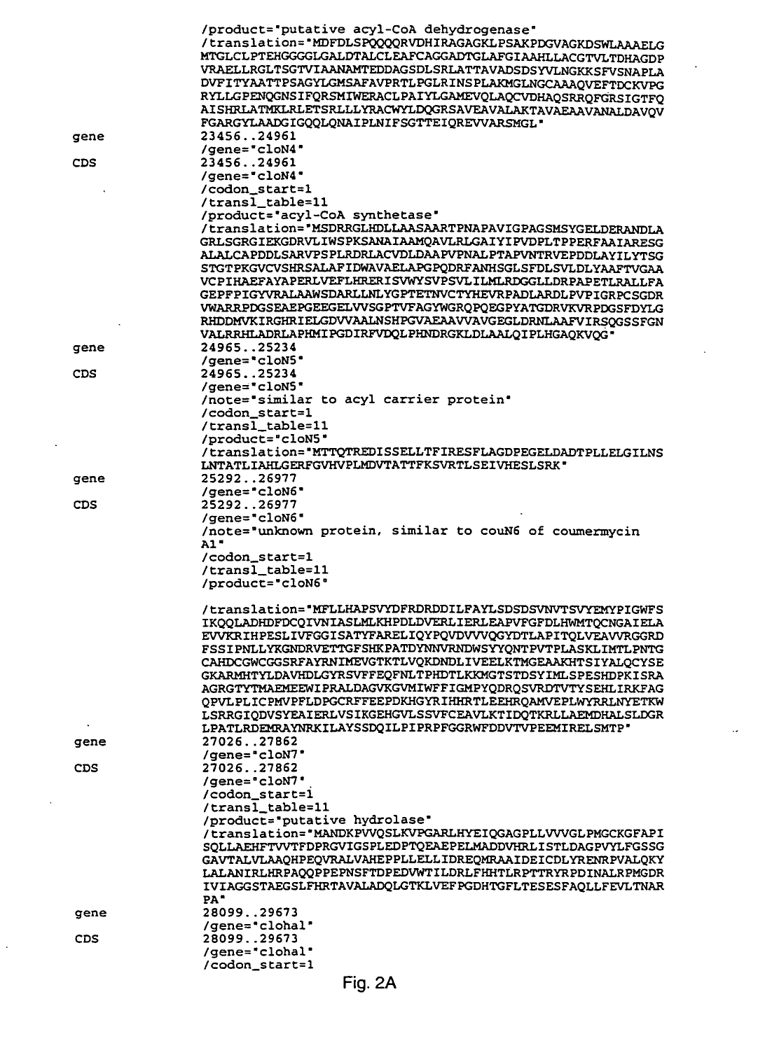

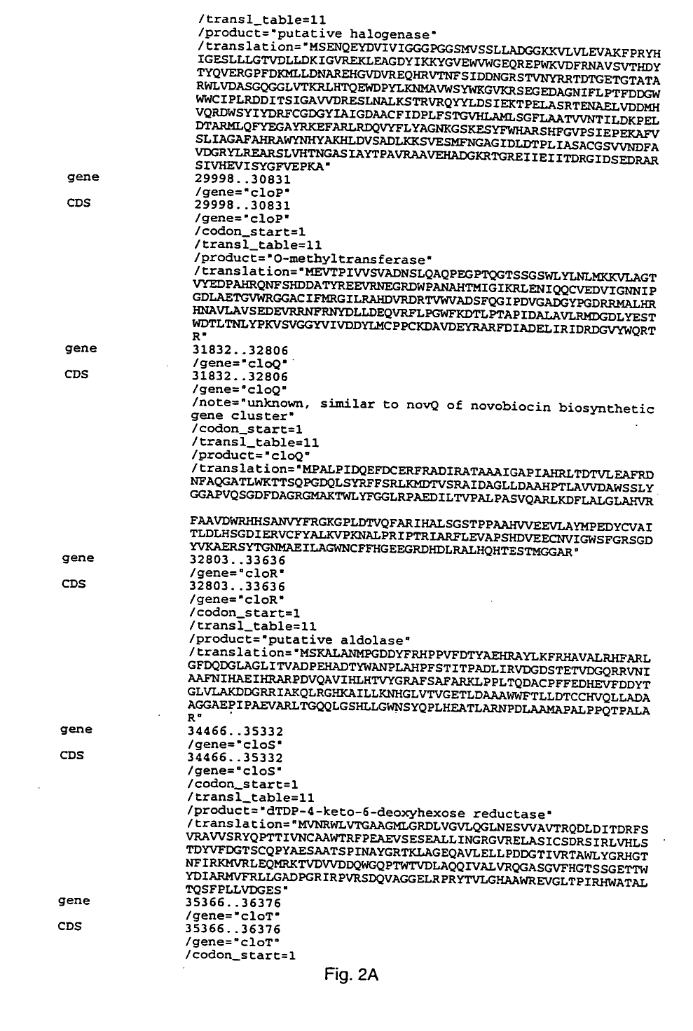

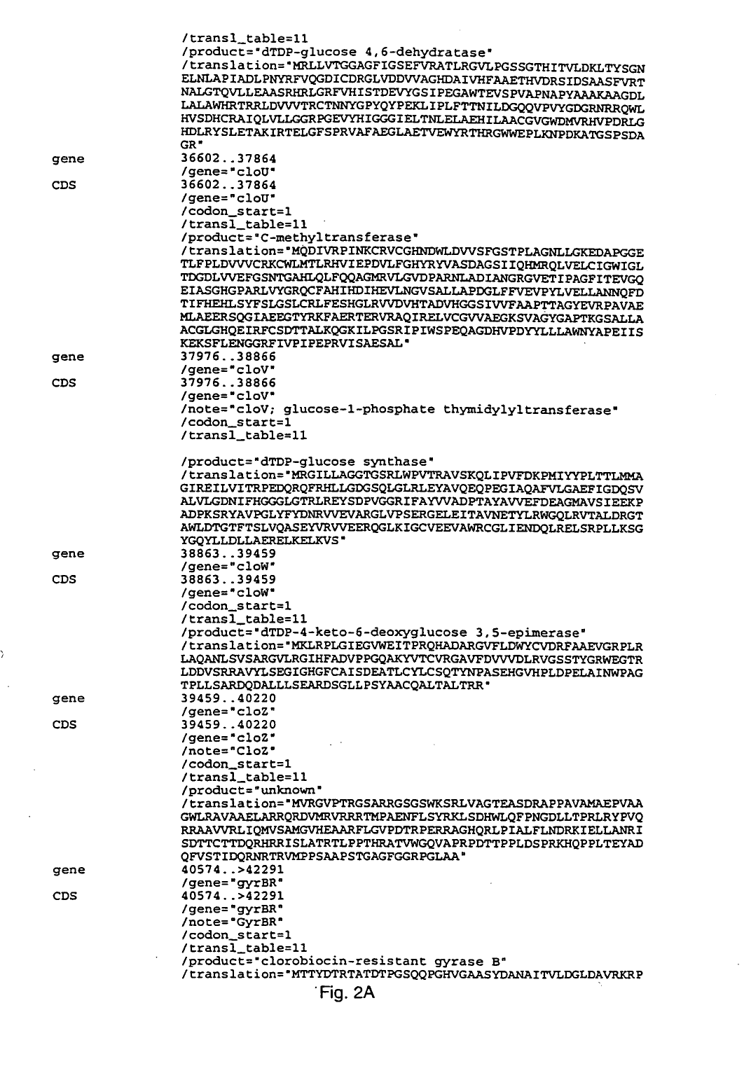

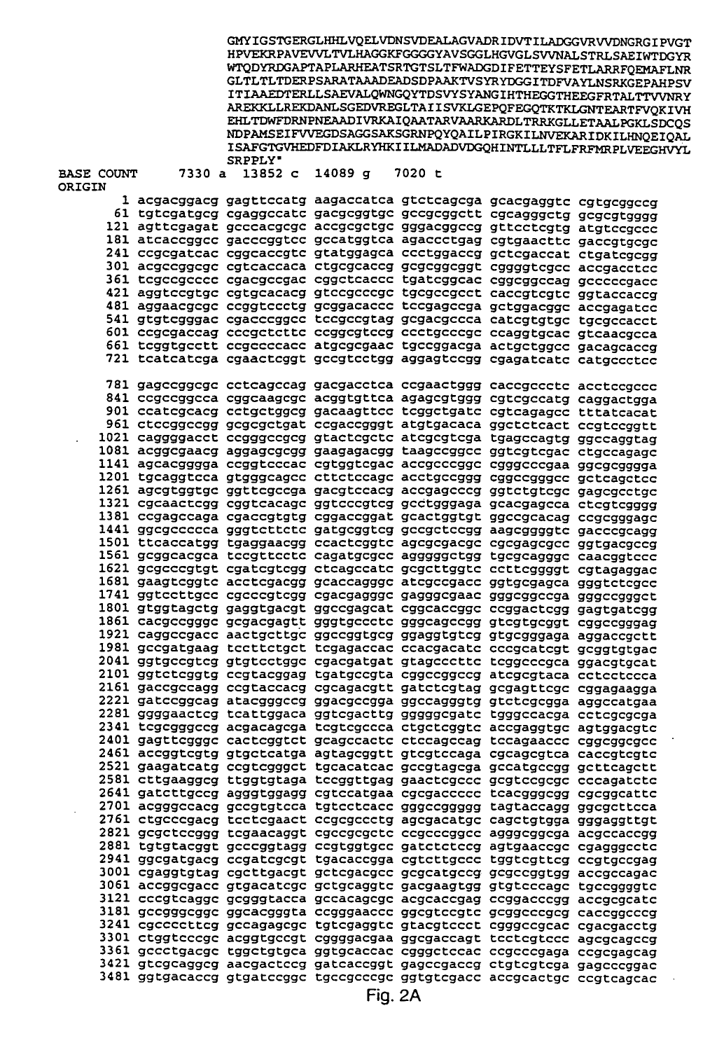

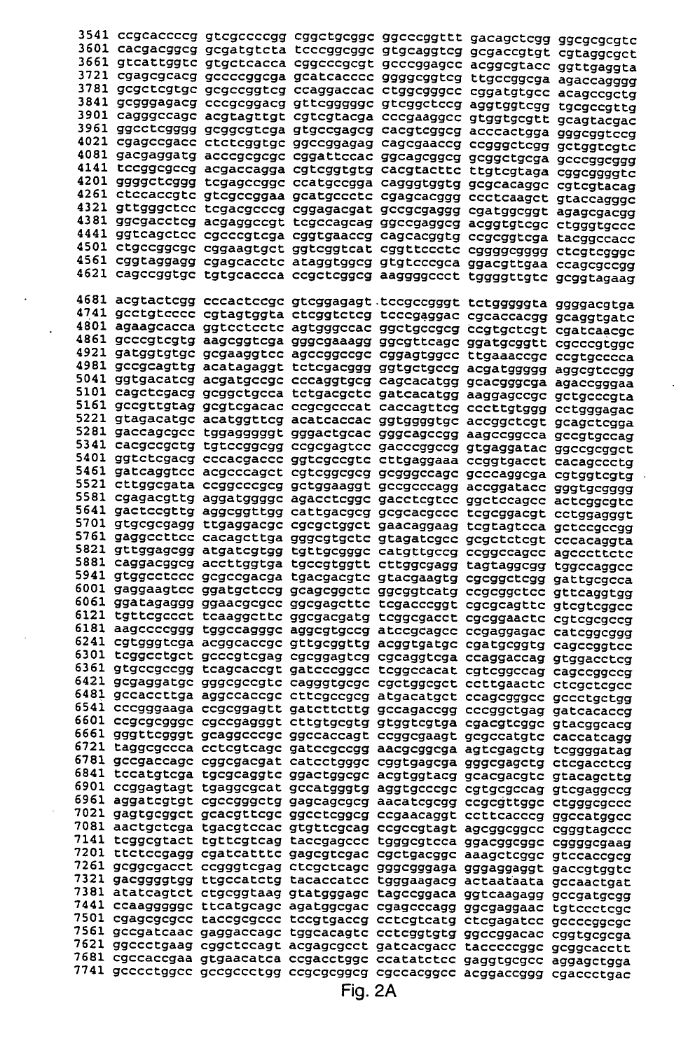

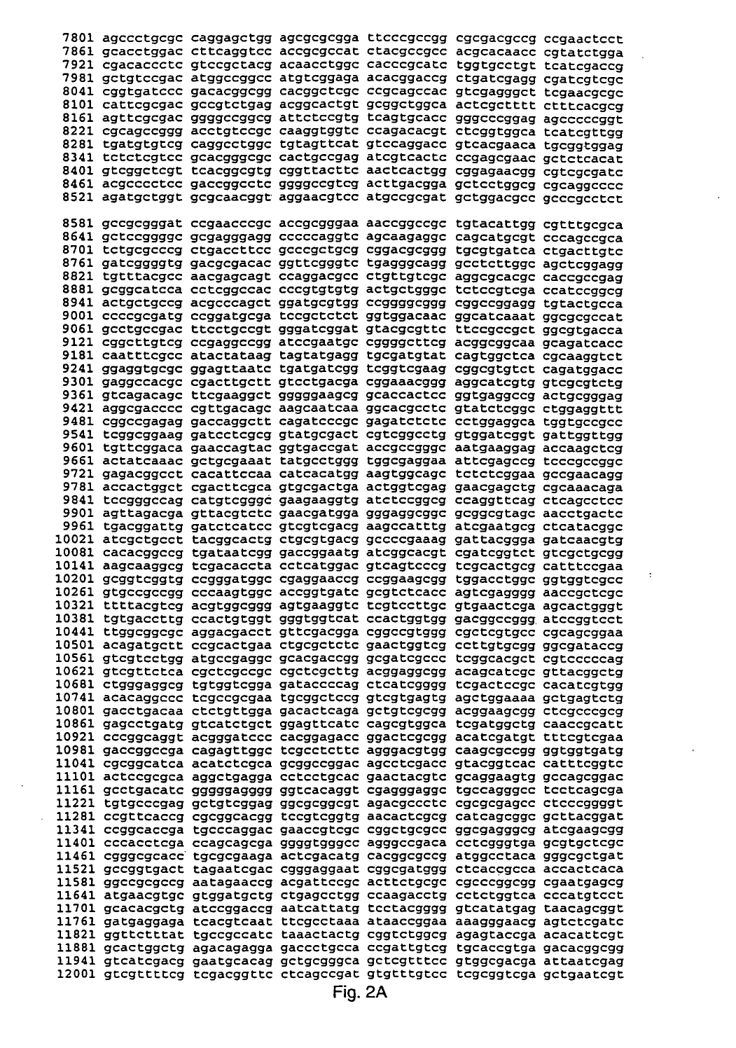

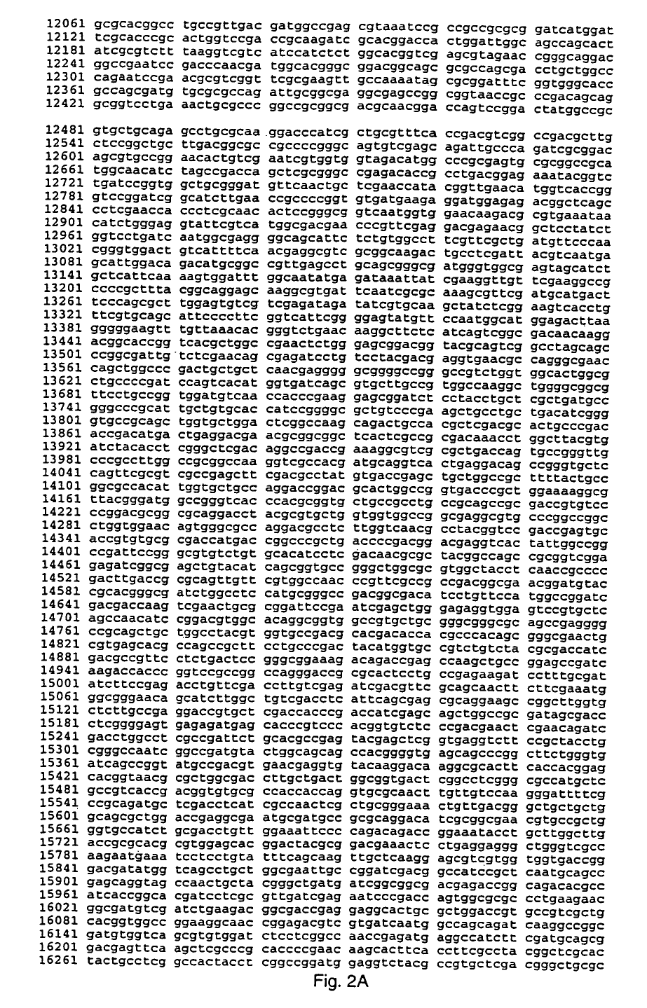

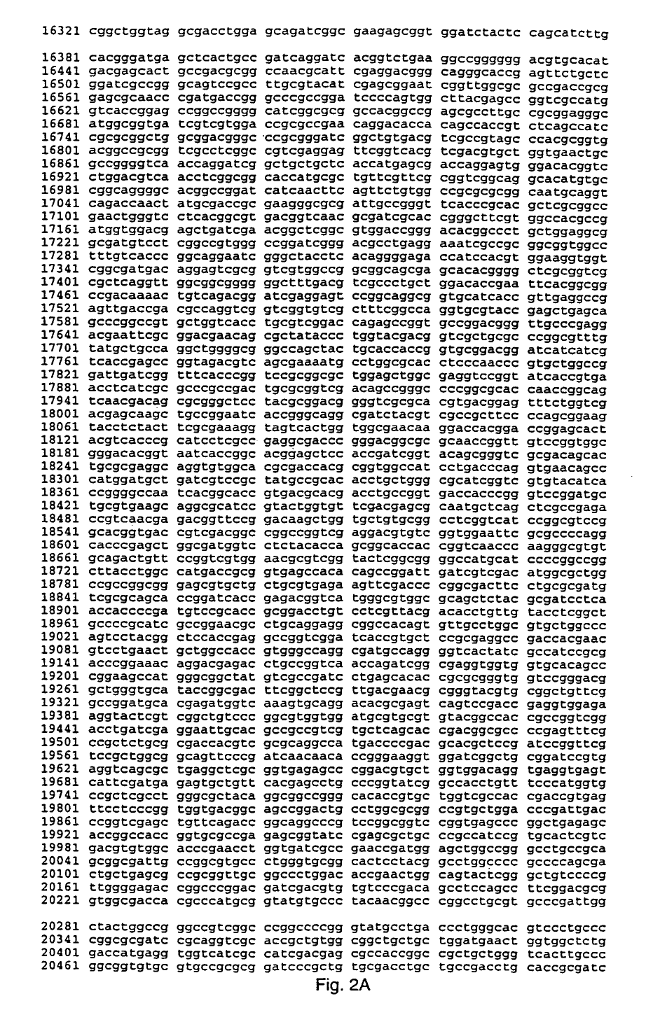

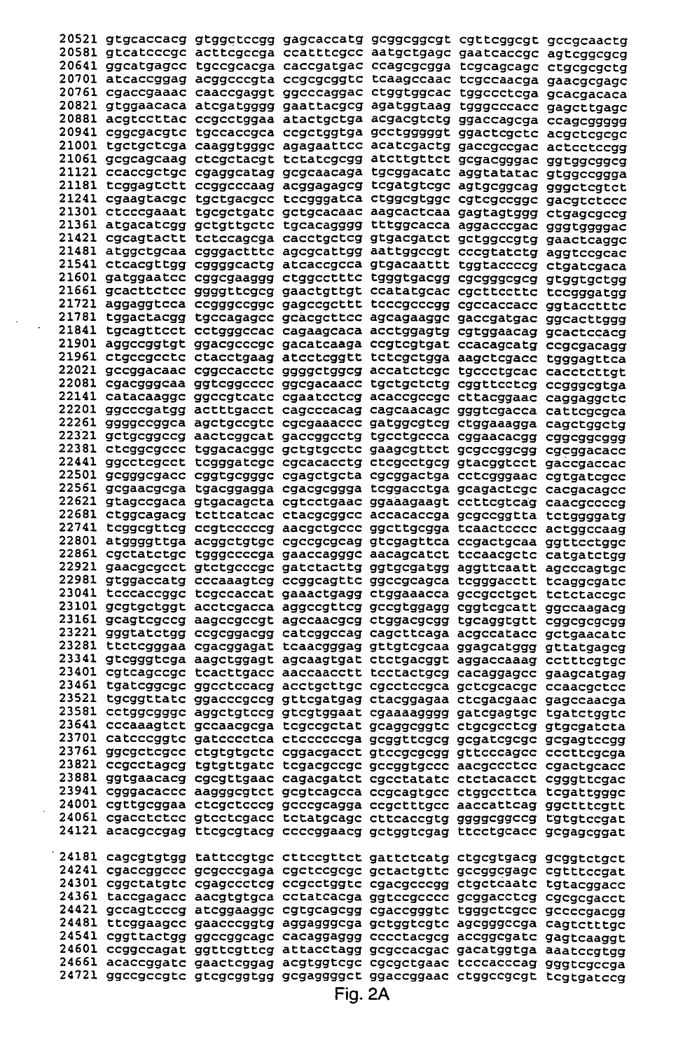

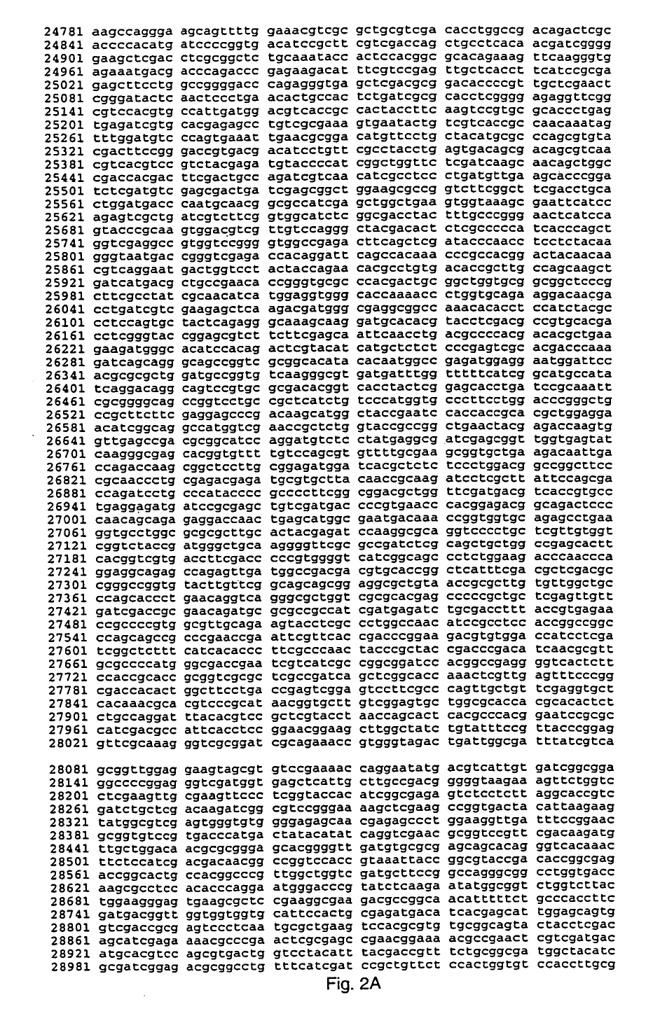

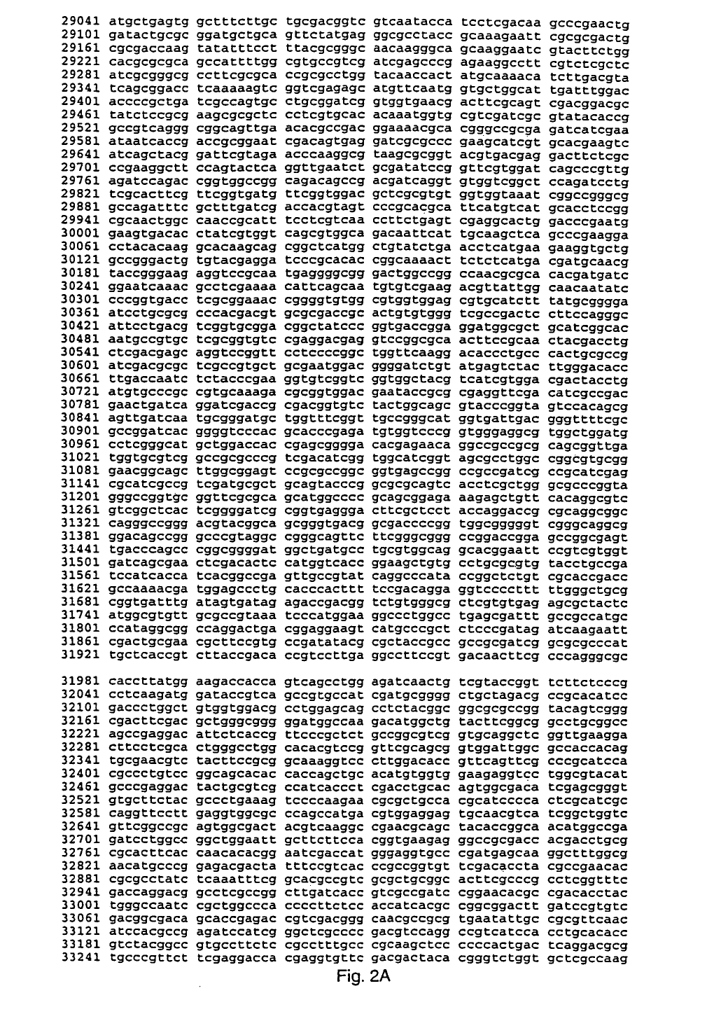

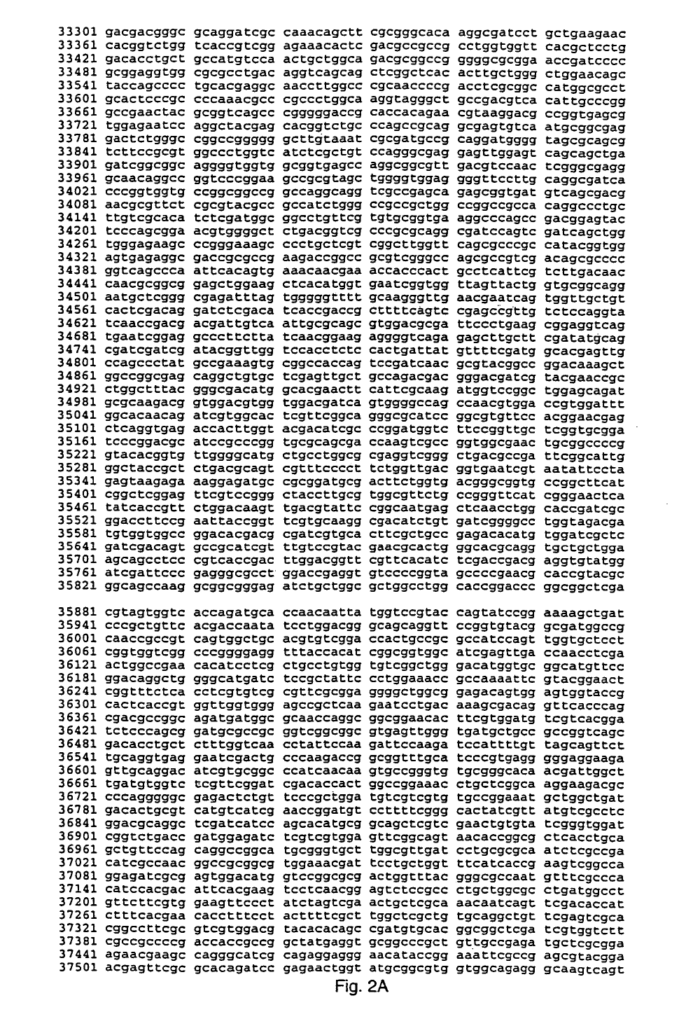

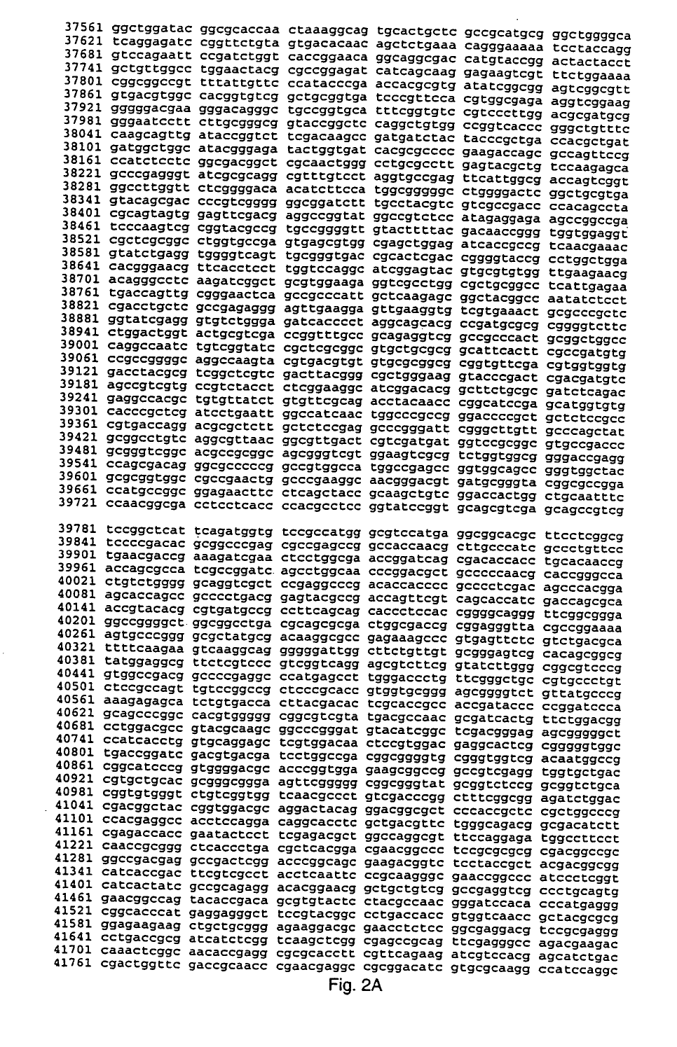

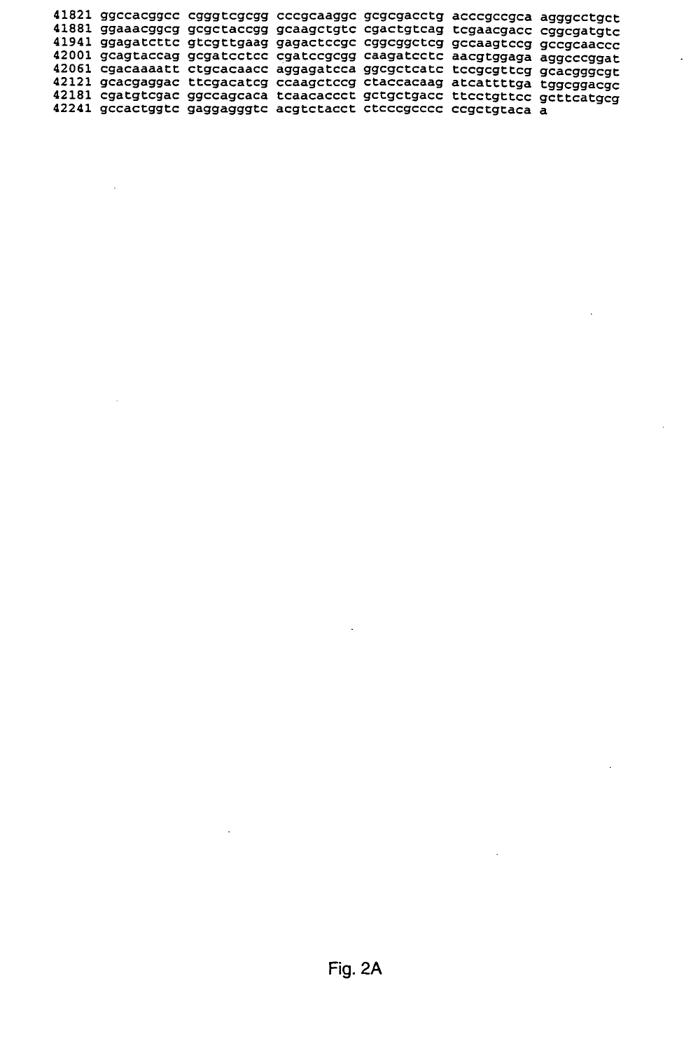

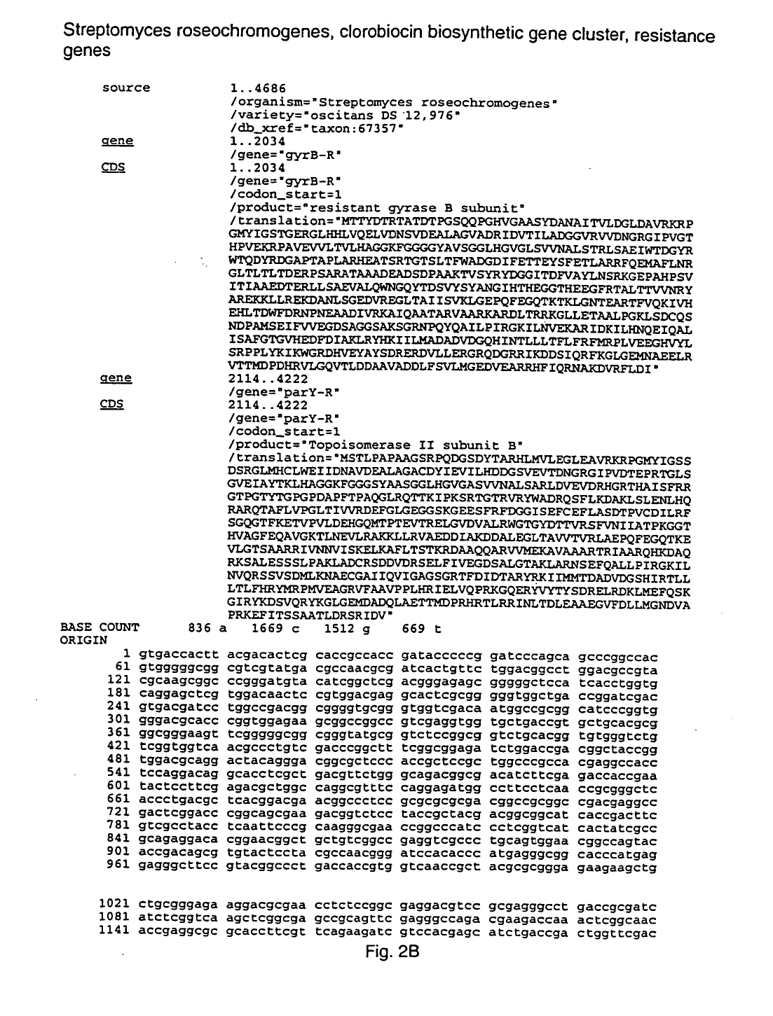

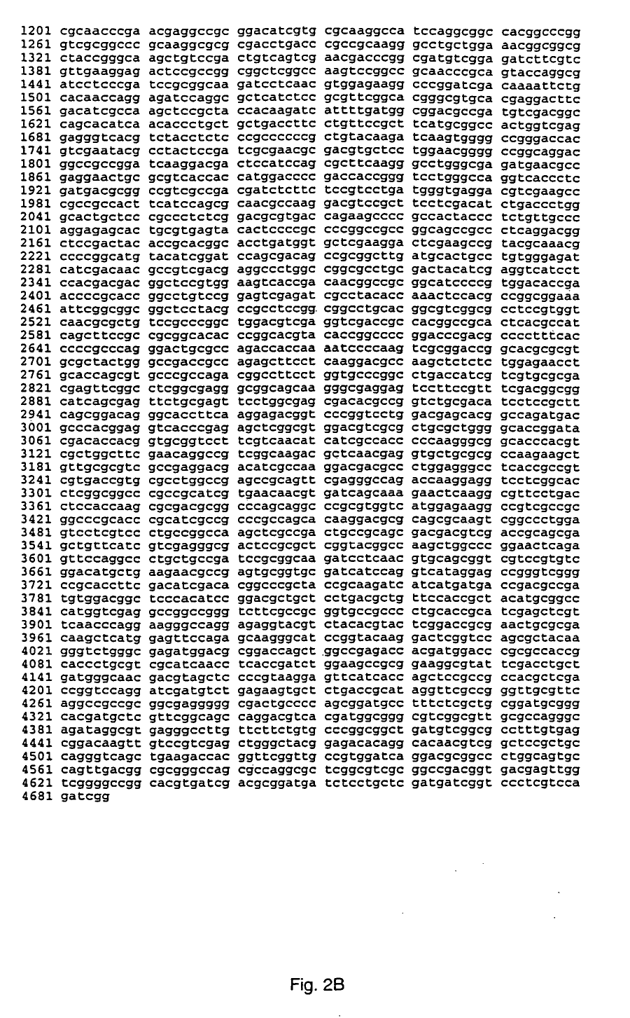

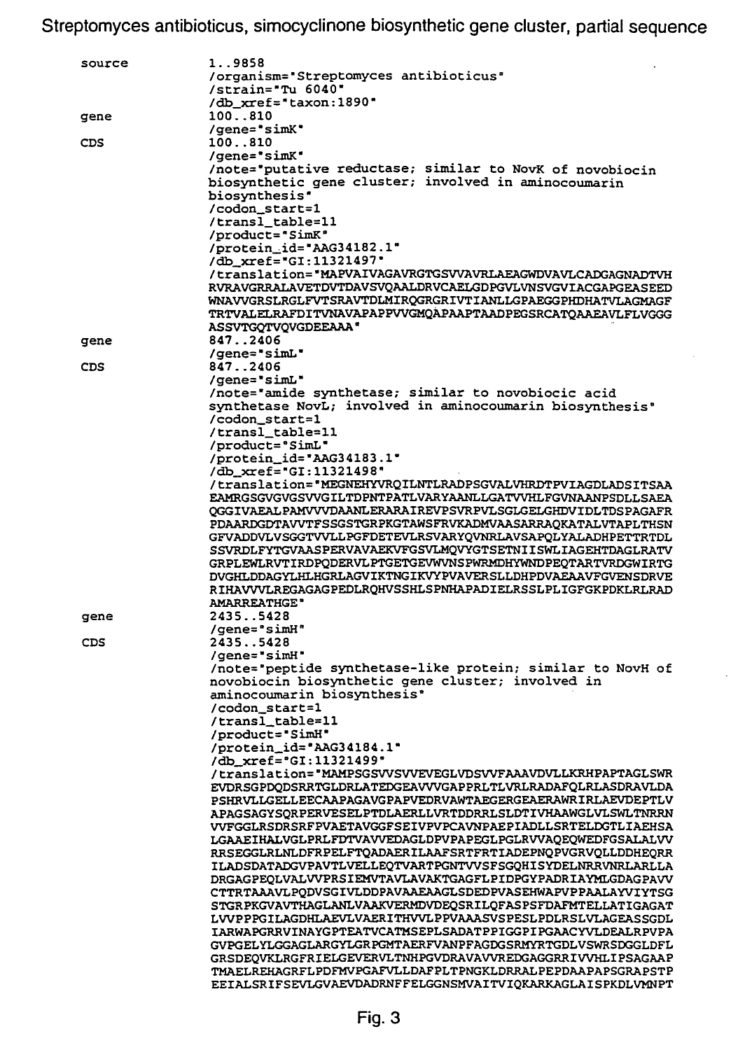

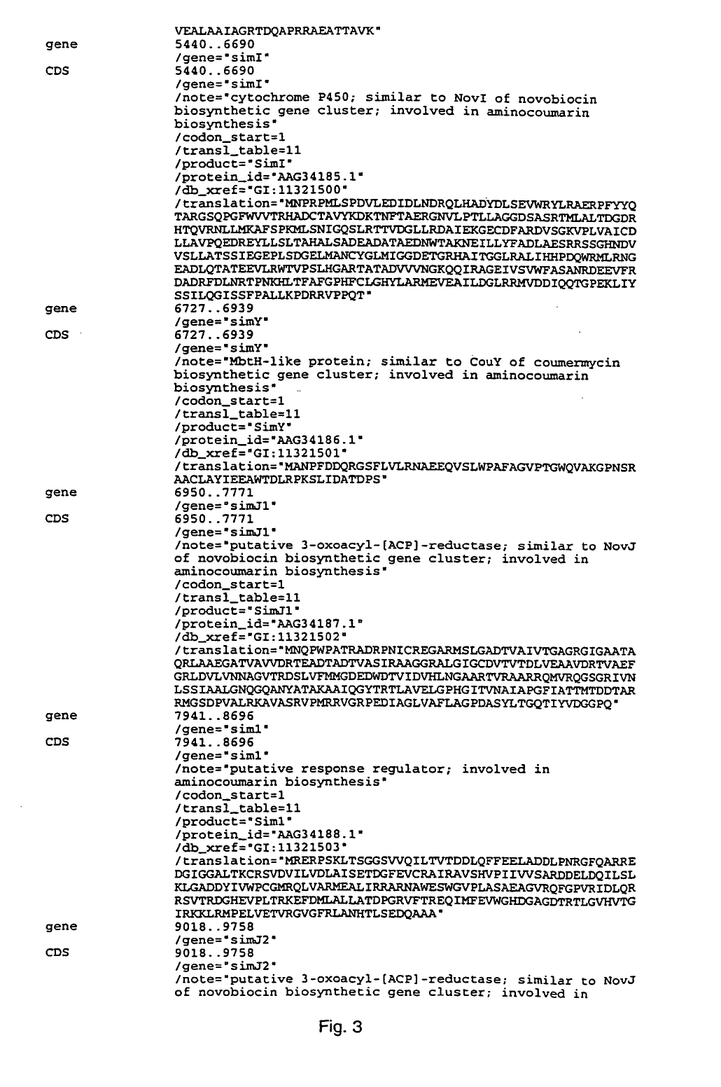

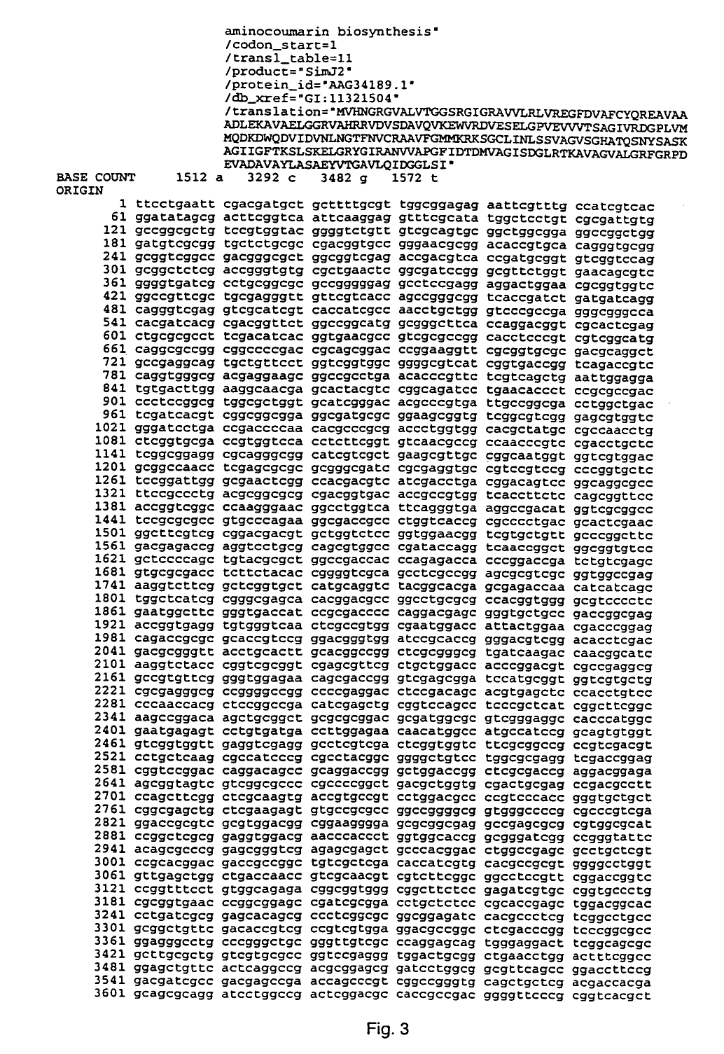

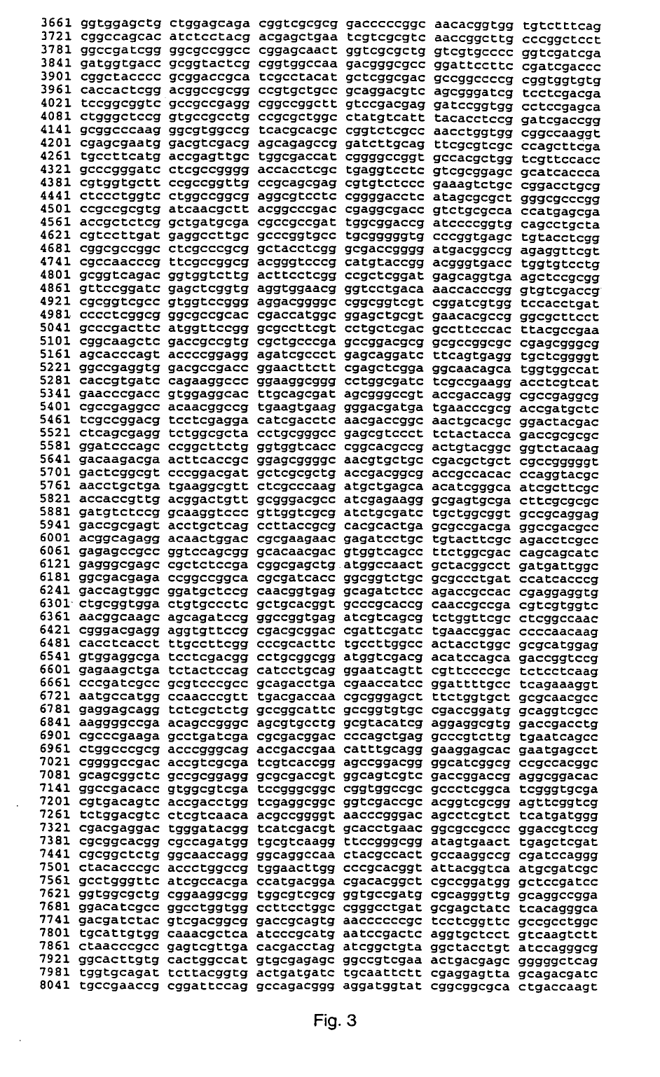

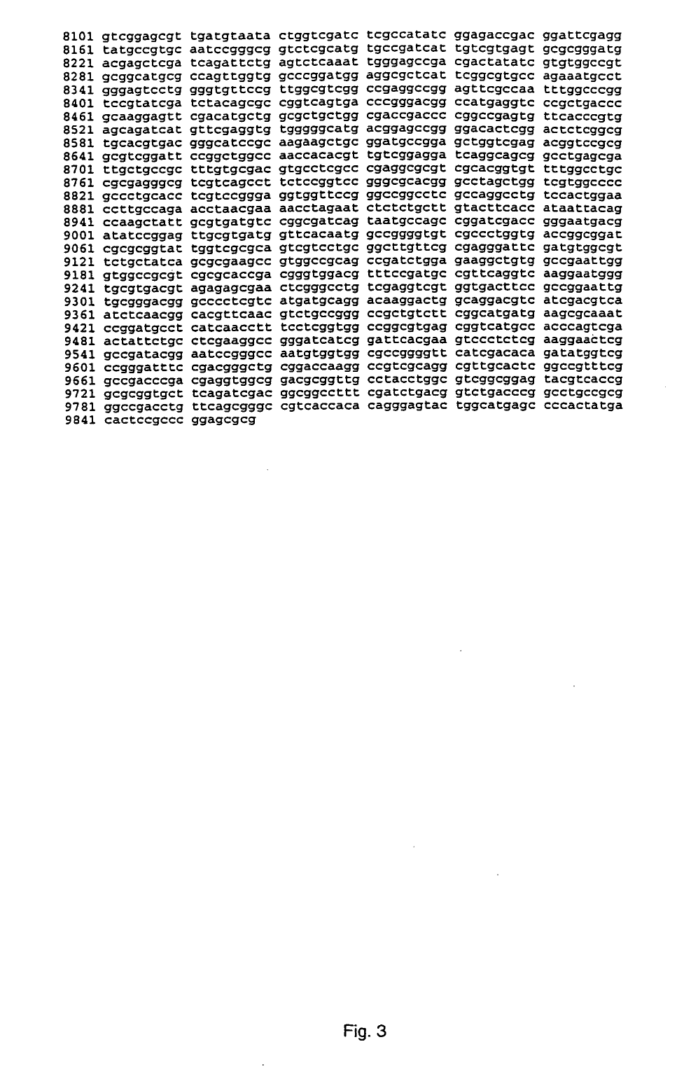

According to a preferred embodiment of the nucleic acid of the present invention, the above nucleotide sequence comprises at least one open reading frame (ORF) contained in the nucleotide sequences shown in FIGS. 1A-1C (coumermycin A₁ biosynthetic gene cluster of Streptomyces rishiriensis), FIGS. 2A and 2B (clorobiocin biosynthetic gene cluster of Streptomyces roseochromogenes) or FIG. 3 (aminocoumarin part of the simocyclinone biosynthetic gene cluster of Streptomyces antibioticus).

The enzyme or functionally active fragment thereof encoded by the nucleotide sequence may also be a mutant enzyme comprising a substitution, addition, insertion and/or deletion of one or more amino acid(s) in comparison to its wild type sequence, thus resulting from a corresponding substitution, addition, insertion and/or deletion of at least one nucleotide in comparison to the wild type nucleotide sequence.

The mutant enzyme may also be a mixture of amino acid sequences encoded by the above aminocoumarin biosynthetic gene clusters.

According to a further preferred embodiment, the nucleotide sequence of the nucleic acid according to the present invention codes for more than one enzyme or functionally active fragment thereof encoded by the above aminocoumarin biosynthetic gene clusters. In particular, it is preferred that the enzymes or functionally active fragments thereof are encoded by different aminocoumarin biosynthetic gene clusters.

A further embodiment of the present invention relates to a vector containing at least the above-defined nucleic acid. The term “vector” refers to a DNA and/or RNA replicon that can be used for the amplification and/or expression of the nucleotide sequence of the nucleic acid or the antisense nucleic acid or the ribozyme as defined above. The vector may contain any useful control units such as promoters, enhancers, or other stretches of sequence within the 5′ and/or 3′ regions of the nucleotide sequence serving for the control of its expression. The vector may additionally contain sequences within the 5′ and/or 3′ region of the nucleotide sequence, that encode amino acid sequences which are useful for the detection and/or isolation of the protein which may be encoded by the nudeotide sequence. Preferably, the vector contains further elements that enable the stable integration of the above-defined nucleic acids into the genome of a host organism and/or the transient expression of the nucleotide sequence of the above-defined nucleic acids. It is also prefered to use vectors containing selectable marker genes which can be easily selected for transformed cells. The necessary operations are well known to the person skilled in the art.

Further subject matter of the present invention relates to a host organism containing the above-defined nucleic acid or the above-defined vector. Examples of suitable host organisms include various eucaryotic and procaryotic cells, such as Bacillus spec. or E. coli, insect cells, plant cells, such as tobacco, potato, or Arabidopsis, animal cells such as vertebrate cell lines, e.g. mammalian cell lines, and fungi such as yeast. Especially preferred bacterial host organisms are, for example, Streptomyces strains such as S. spheroides, S. niveus, S. roseochromogenes, S. rishiriensis, S. antibioticus and S. lividans.

A further embodiment of the present invention relates to a polypeptide encoded by an ORF of an aminocoumarin biosynthetic gene cluster, wherein said aminocoumarin is selected from the group consisting of coumermycin A₁, clorobiocin and simocyclinone. Preferred examples of the polypeptide according to the present invention contain (an) amino acid sequence(s) shown in FIGS. 1 to 3.

The genetic information for the biosynthesis of aminocoumarins such as novobiocin, clorobiocin, coumermycin A₁ and simocyclinone is particularly useful for the production of modified aminocoumarins based on said known molecules. Therefore, the present invention also relates to a method for the production of a modified aminocoumarin selected from the group consisting of novobiocin, clorobiocin, coumermycin A₁ and simocyclinone, said method comprising the steps of:
(a) inactivating at least one gene of the gene cluster for the biosynthesis of one aminocoumarin in an organism containing said gene cluster;
(b) introducing into said organism at least one biosynthetic gene from another organism and/or feeding said organism with an analogue of a structural moiety of an aminocoumarin;
(c) cultivating said organism containing said gene cluster in a suitable medium; and
(d) isolating the aminocoumarin produced by said organism.

Preferrably, said at least one biosynthetic gene from another organism is a gene of the biosynthetic gene cluster of another aminocoumarin.

According to a further preferred embodiment of the above-defined method, the organism containing the aminocoumarin biosynthetic gene cluster is selected from the group of consisiting of S. spheroides, S. niveus, S. roseochromogenes, S. rishiriensis and S. antibioticus.

Therefore, the method according to the present invention preferably provides hybrid antibiotics based on the above aminocoumarins by combination of the various regions of the biosynthetic gene clusters. A “hybrid antibiotic” means that one or more structural elements of the aminocoumarins (see FIG. 5) is/are exchanged. For example, the method according to the present invention may be used to exchange the carbamoyl group of novobiocin with the 5-methyl-pyrol-2-carboxylic acid unit of coumermycin A₁ or of clorobiocin and vice versa.

A further preferred example is the exchange of the chlorine atom of clorobiocin with the corresponding methyl group of coumermycin A₁ or novobiocin and vice versa.

According to the method of the present invention, a defect mutant is produced by inactivating one or more genes of the biosynthetic gene cluster for the biosynthesis of an aminocoumarin, for example by insertional inactivation. Then, the thus obtained defect mutant is transformed with another biosynthetic gene, preferably with one or more corresponding gene(s) derived from another aminocoumarin biosynthetic gene cluster.

The general principle of such combinatorial biosynthesis is known to a person skilled in the art (Hutchinson (1998) Curr. Opin. Micriobiol. 1: 319-329).

A further possibility for the production of novel antibiotics using the genetic information of aminocoumarin biosynthetic gene clusters is to obtain an analogue of a structural moiety of an aminocoumarin by a chemical synthesis, for example a structural analogue of the prenylated 4-hydroxybenzoic acid unit and then feeding a defect mutant, for example a mutant of a clorobiocin-producing Streptomyces strain being blocked in the biosynthesis of the prenylated 4-hydroxy benzoic acid, with said structural analogue.

The novel antibiotics obtained by the method according to the present invention are isolated according to methods well known to a person skilled in the art and may then be tested for affinity to bacterial DNA gyrase. Furthermore, the compounds are tested against multi-resistant strains of, for example, Staphylococcus aureus. Infections with S. aureus represent an important target for the therapeutic application of aminocoumarin antibiotics, since there is a substantial need for novel therapies of infections by S. aureus strains displaying multi-resistancies, for example against vancomycin.

Thus, the present invention further relates to a pharmaceutical composition containing the aminocoumarin compound of the present invention in a pharmaceutically effective amount, optionally in combination with a pharmaceutically acceptable carrier and/or diluent. Suitable carriers and diluents are well known to a person skilled in the art.

The pharmaceutical composition may preferably be used in the treatment of infections with gram-positive bacteria and of malignant diseases. Especially in the treatment of malignancies, it is preferred that the pharmaceutical composition further contains a pharmaceutically effective amount of a cytostatic agent, in particular a podophyllotoxin derivative such as etiposide, teniposide and mitopodozid.

Furthermore, the present invention provides a method for treating a patient comprising the step of administering to said patient a pharmaceutically effective amount of the above-defined aminocoumarin compound.

The Figures show:

FIG. 1 shows (A) the genetic information contained in the coumermycin A₁ biosynthetic gene cluster; partial sequence (SEQ-ID-No. 1); (B) the genetic information contained in the coumermycin A₁ biosynthetic gene cluster; complete sequence (SEQ-ID-No. 2); and (C) the genetic information contained in the coumermycin A₁ biosynthetic gene cluster, resistence genes (SEQ-ID-No. 3);

FIG. 2 shows (A) the genetic information contained in the clorobiocin biosynthetic gene cluster; complete sequence (SEQ-ID-No.4); and (B) the genetic information contained in the clorobiocin biosynthetic gene cluster; resistence genes (SEQ-ID-No.5);

FIG. 3 shows the genetic information contained in the aminocoumarin part of the simocyclinone biosynthetic gene cluster (SEQ-ID-No.6);

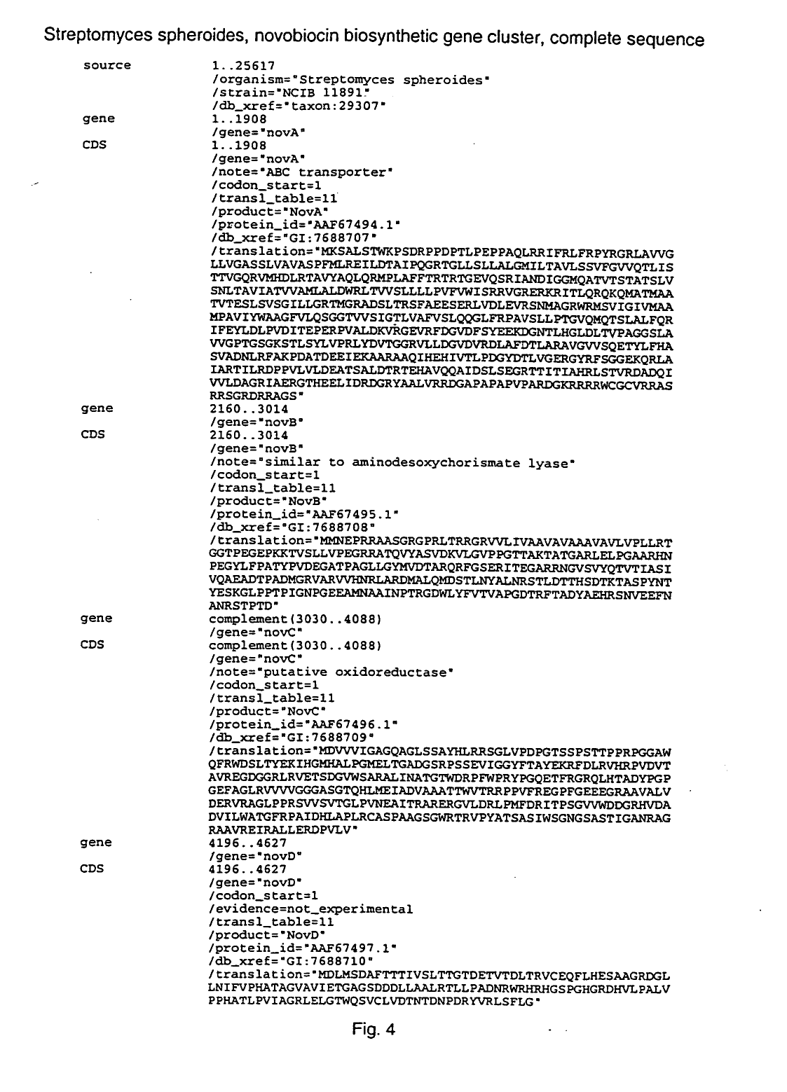

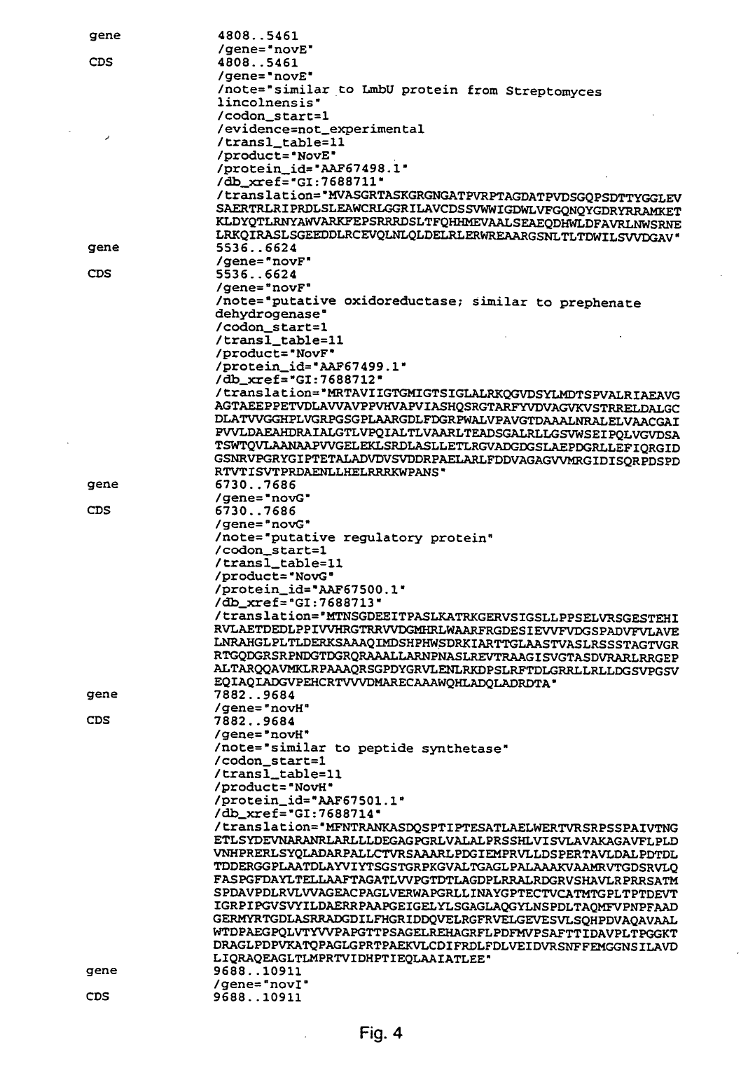

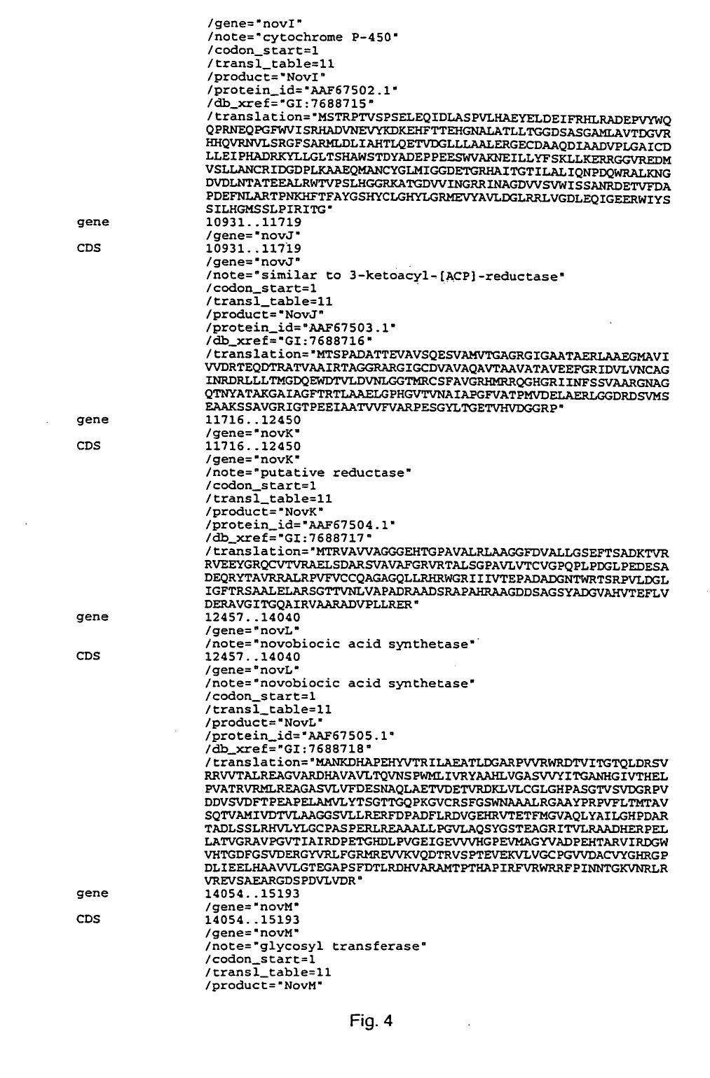

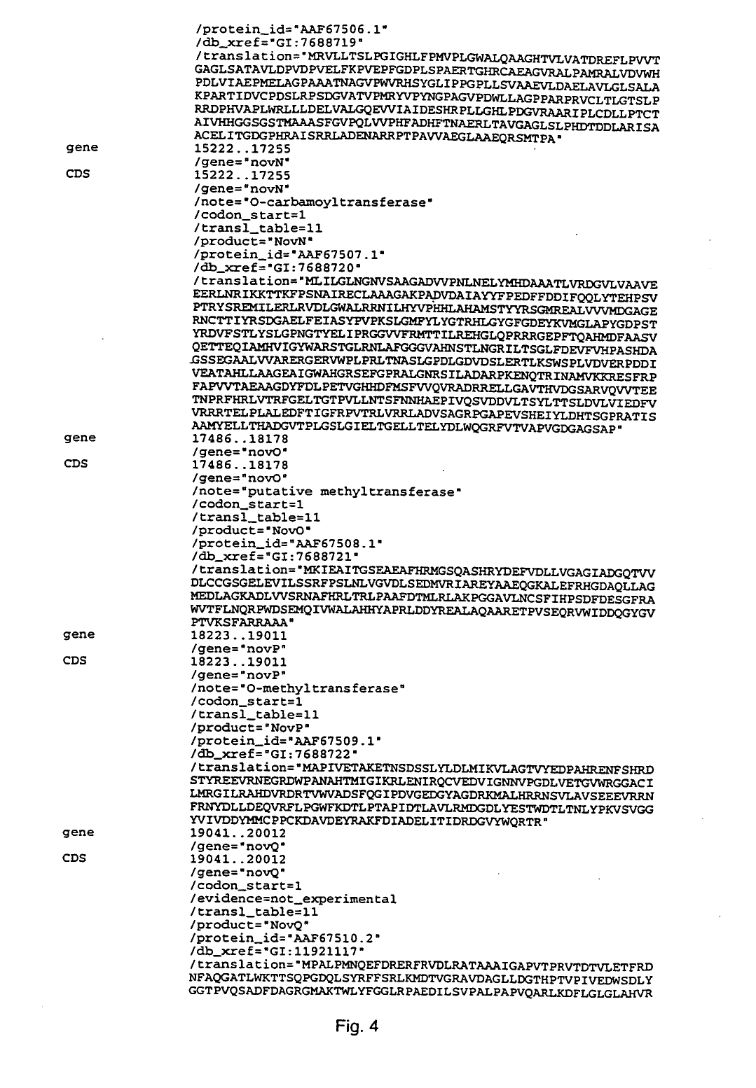

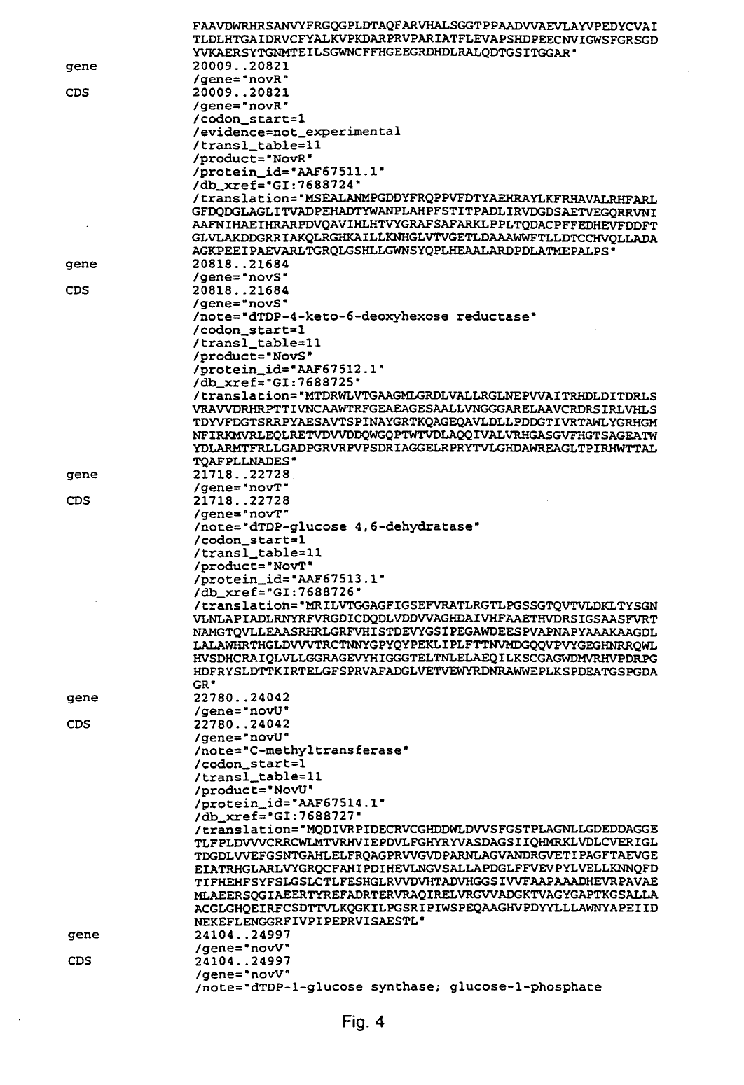

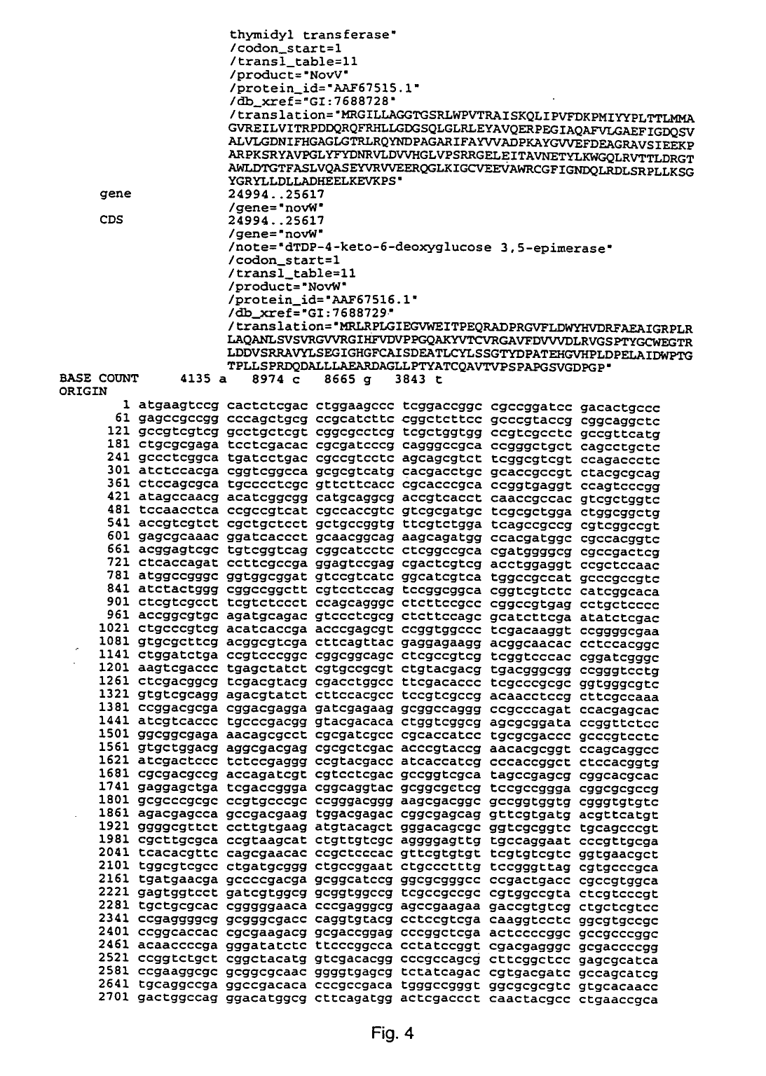

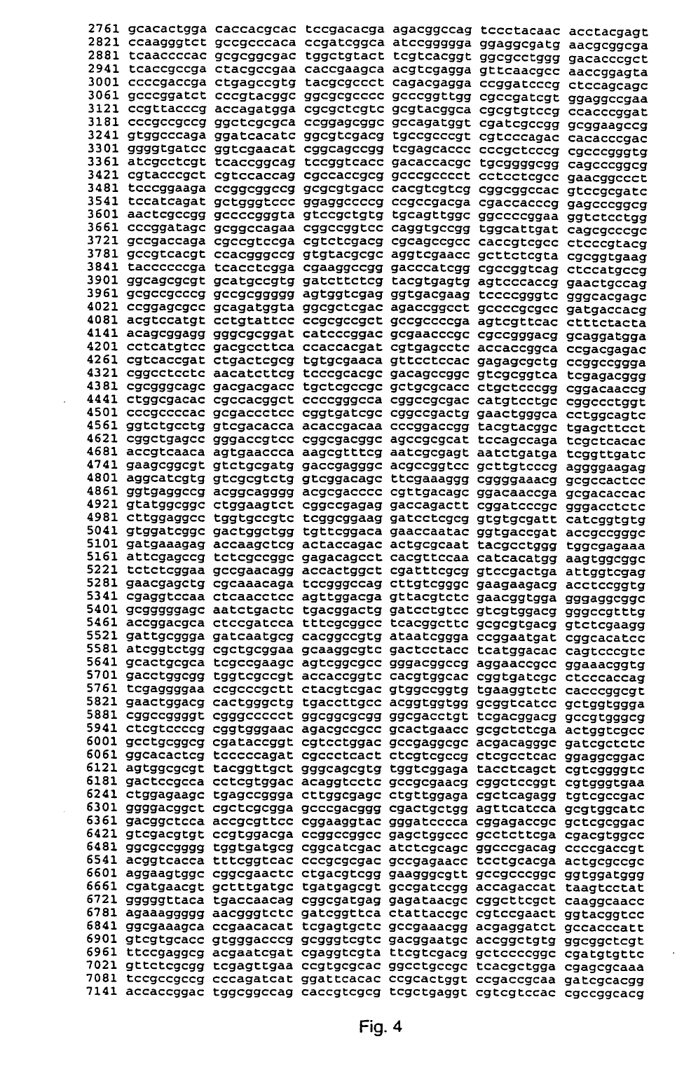

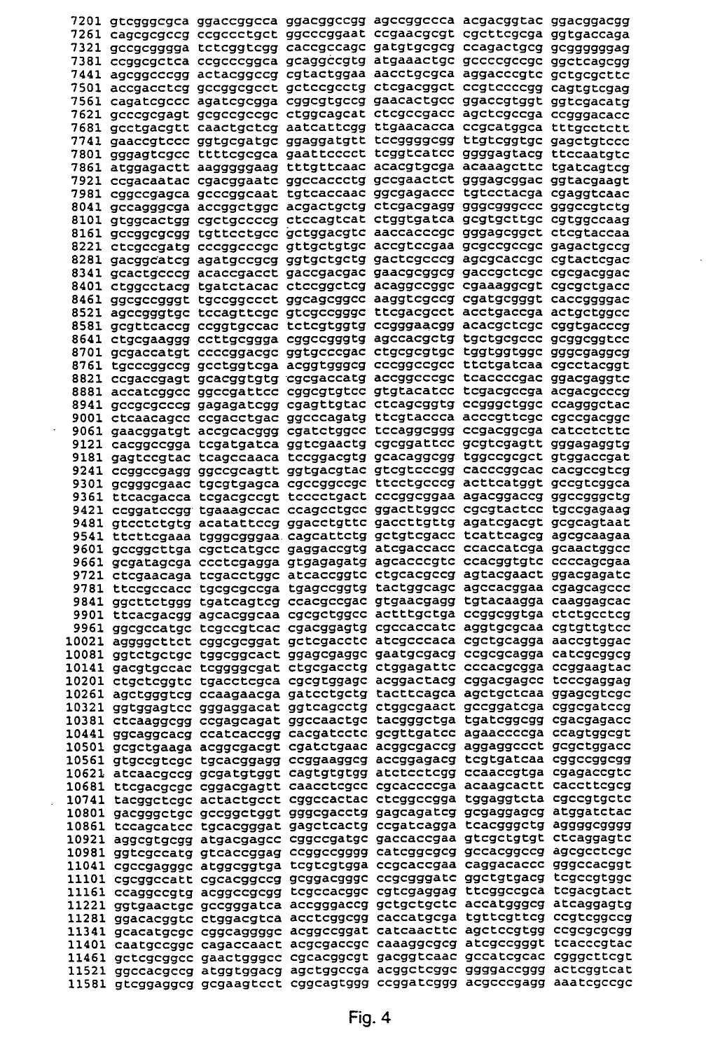

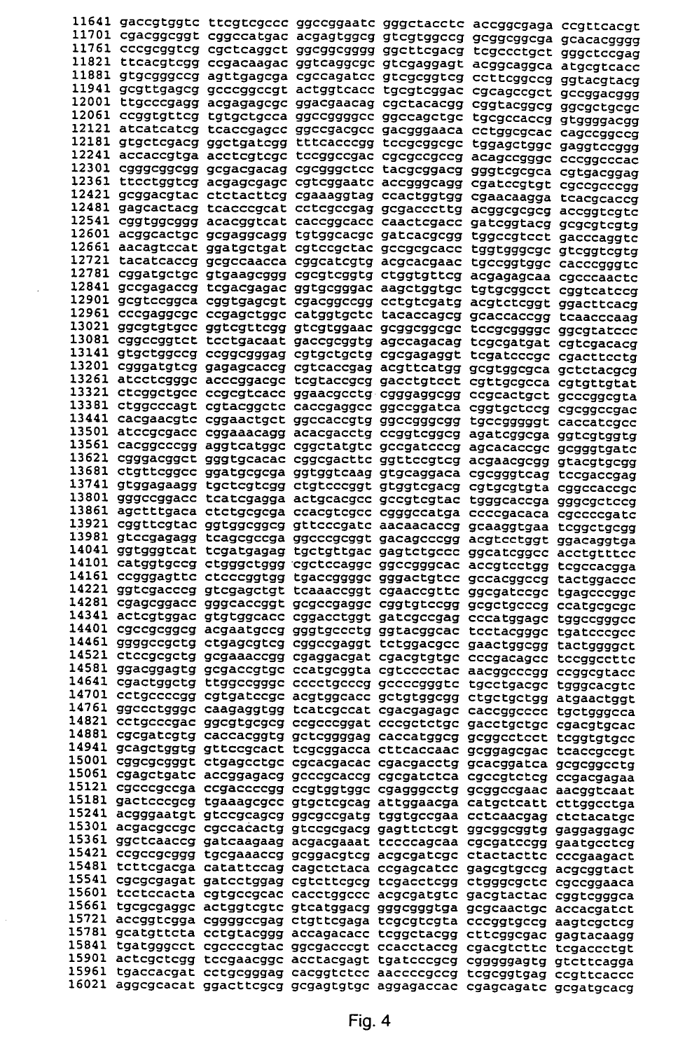

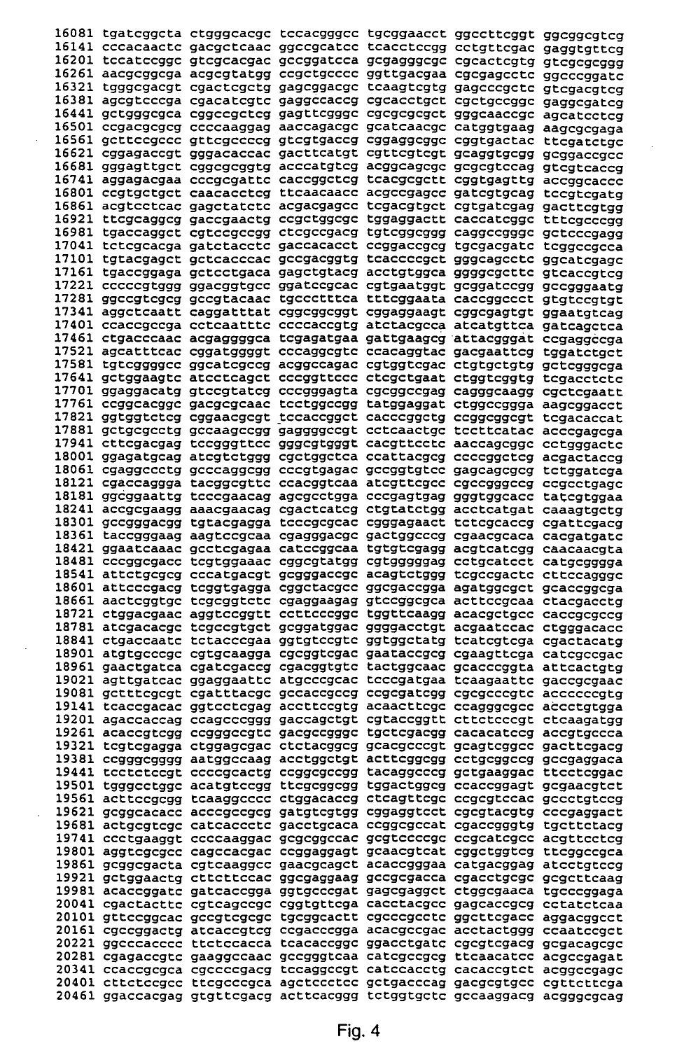

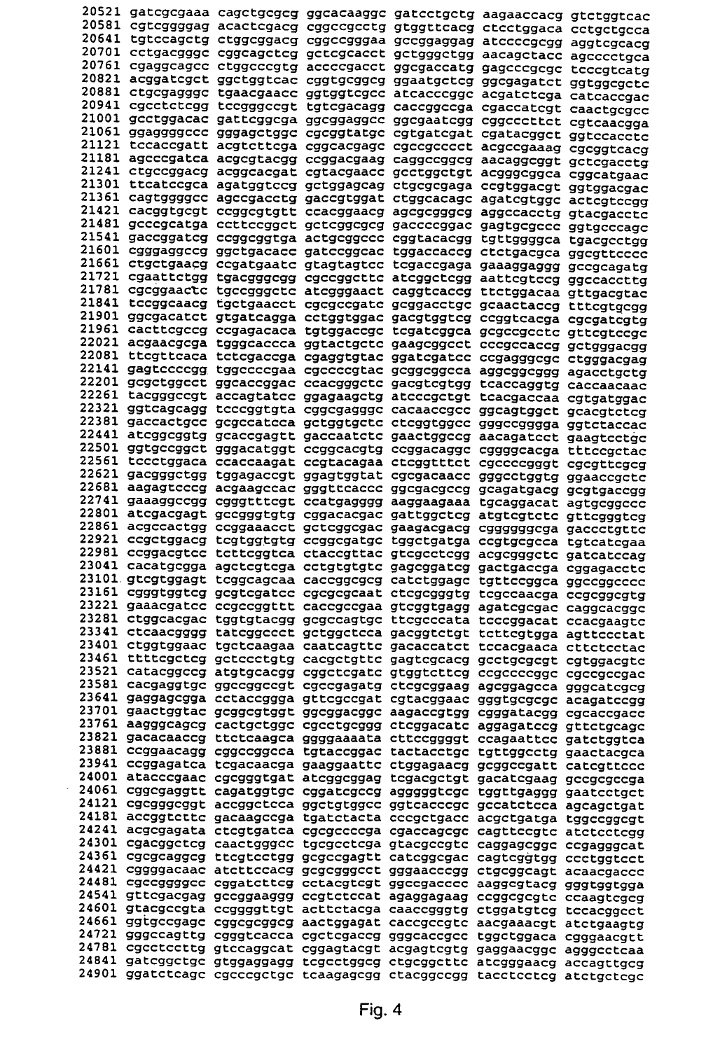

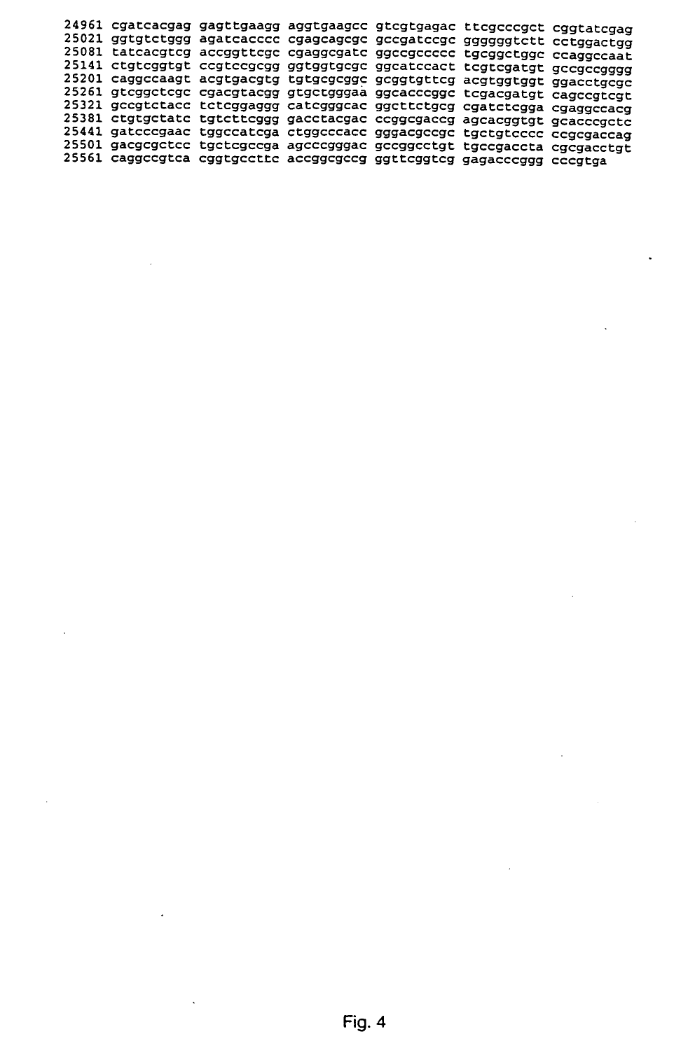

FIG. 4 shows the genetic information contained in the novobiocin biosynthetic gene cluster as published in Steffensky et al. (2000) (SEQ-ID-No.7);

FIG. 5 shows the structures of novobiocin, coumermycin A₁, clorobiocin and simocyclinone;

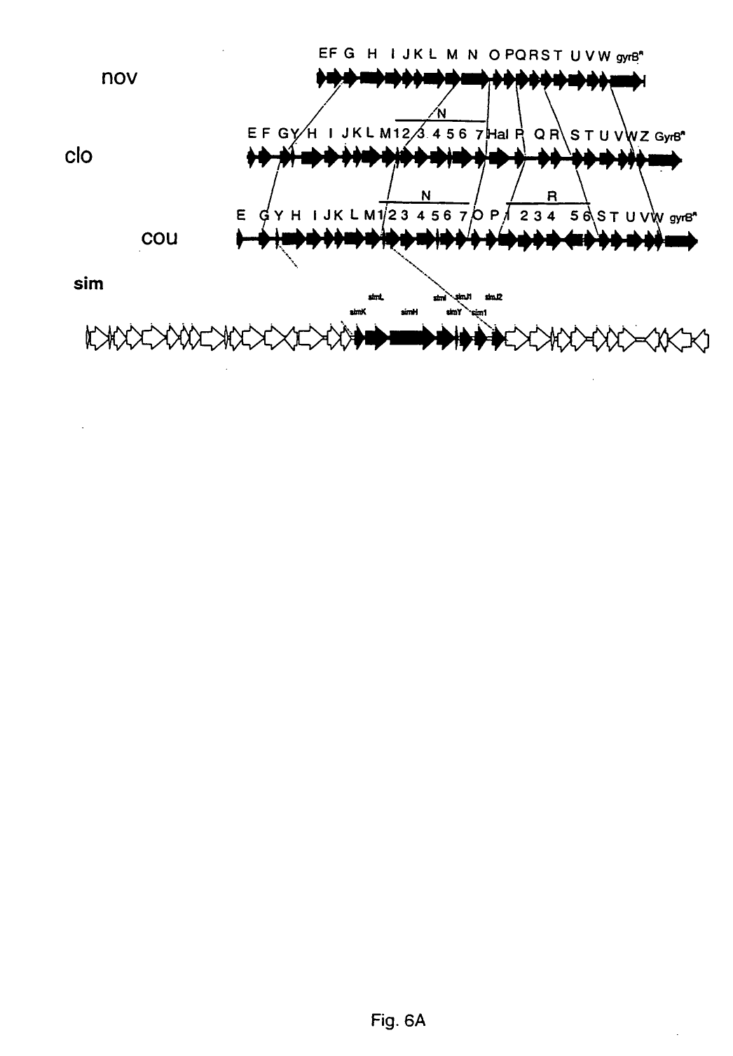

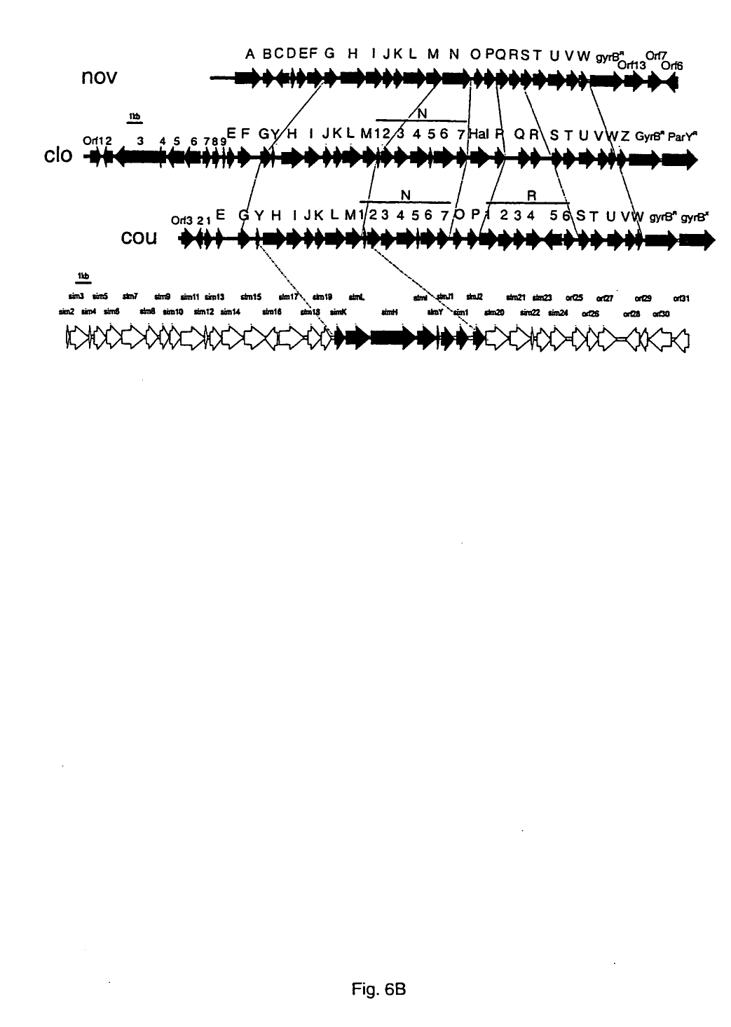

FIG. 6 shows (A) a schematic representation of the biosynthetic gene clusters of novobiocin (nov), clorobiocin (clo), coumermycin A₁ (cou) and simocyclinone (sim; aminocoumarin part); partial sequence; and (B) a schematic representation of the biosynthetic gene clusters of novobiocin (nov), clorobiocin (clo), coumermycin A1 (cou) and simocyclinone (sim; aminocoumarin part), complete sequence;

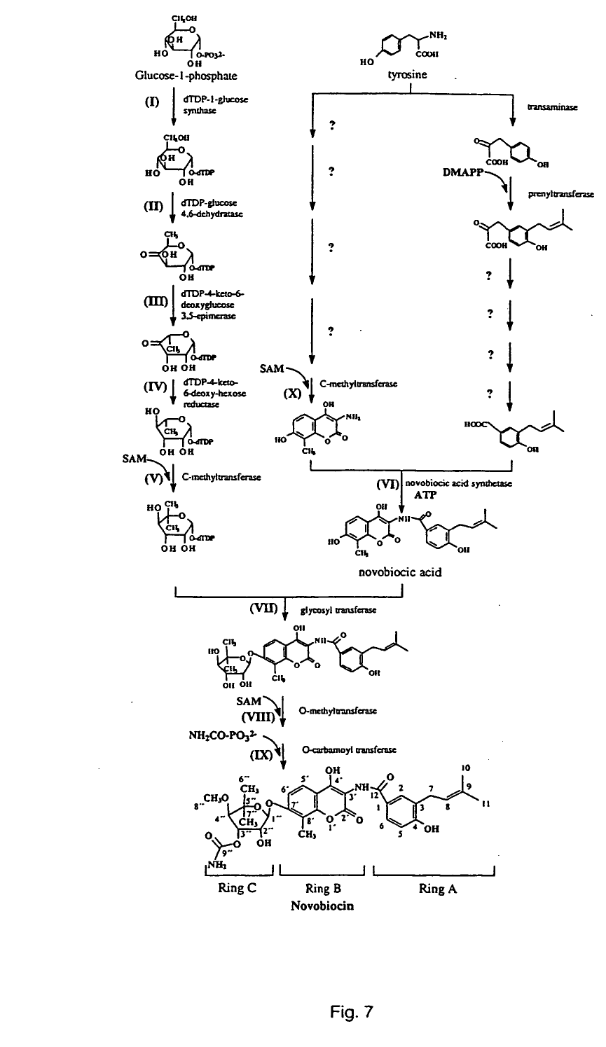

FIG. 7 is a scheme showing the (hypothetical) biosynthetic pathway of novobiocin in Streptomyces spheroides (see Steffensky et al. (2000);

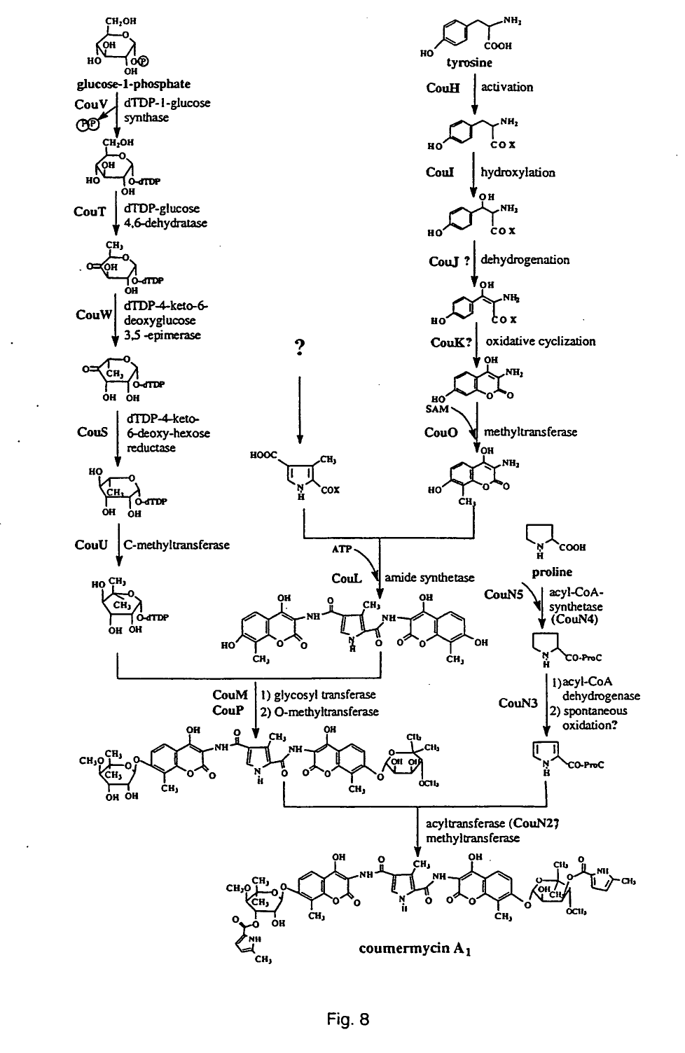

FIG. 8 is a scheme showing the (hypothetical) biosynthetic pathway of coumermycin A₁ in Streptomyces richeriensis;

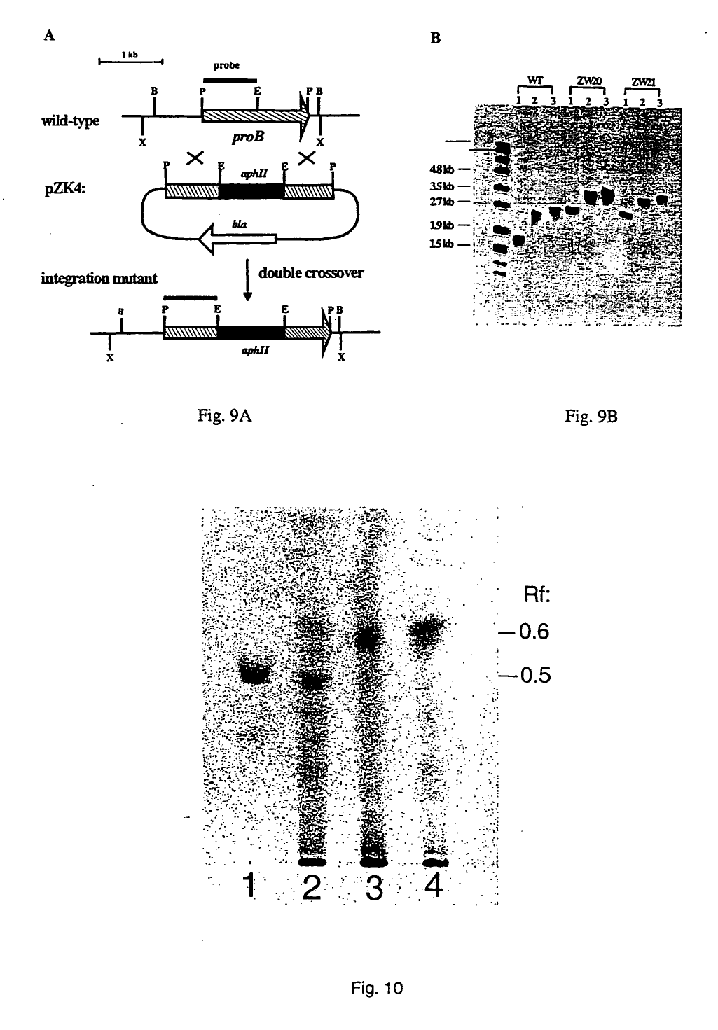

FIG. 9 illustrates an insertional gene inactivation experiment in the coumermycin A₁ biosynthetic gene cluster. (A) Schematic representation of the gene replacment; the 0.87 kB DNA fragment used is indicated as a black line; relevant restriction sides: P, Pstl; B, BamHI; X, Xhol; E, EcoRI. aphll, neomycin resistance gene. (B) Southern blot analysis of the couN4 (proB) mutants. Lanes 1, Pstl; lanes 2, BamHI; lanes 3, Xhol. Expected Bands: wild-type 1.66 kb (Pstl), 2.45 kb (BamHI), 2.66 kb (Xhol); mutants resulting from gene replacement: 2.65 kb (Pstl), 3.44 kb (BamHI), 3.65 kb (Xhol).

FIG. 10 is a photographic reproduction of a thin-layer chromatographic analysis of secondary metabolites in S. risheriensis strains; Lane 1, coumermycin A₁ standard; lane 2, extract of DSM 40489 (wild type); lanes 3 and 4, extracts of couN4 (proB)-mutants ZW20 and ZW21. The plate (silica gel 60 F₂₅₄) was developed with dichloromethane-methanol-formic acid (45:2:1). Blue spots on yellow background were observed under daylight after spraying with fresh 10% ferric chloride-potassium ferricyanide (1:1).

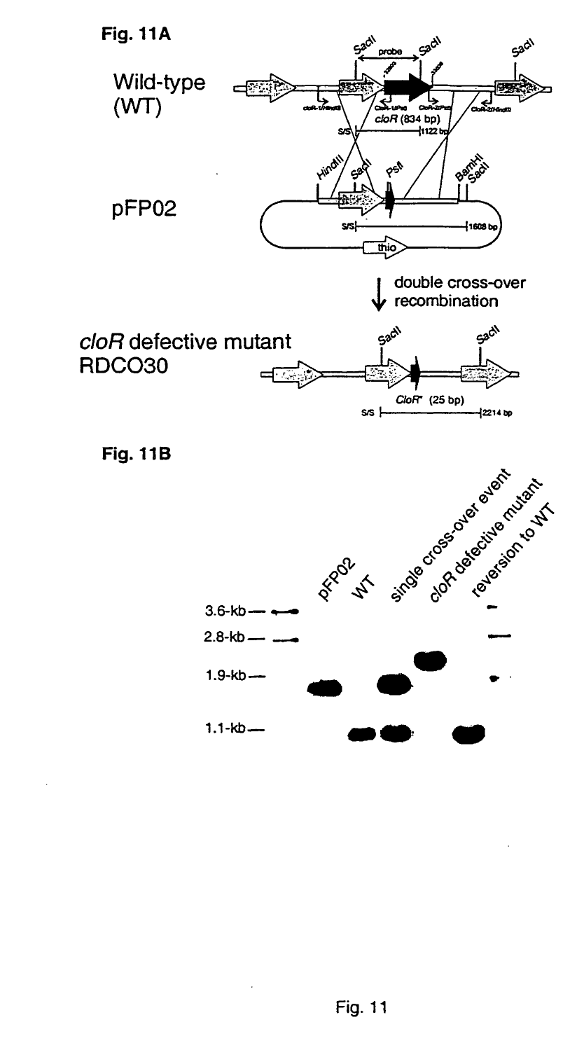

FIG. 11 shows an inactivation of the gene cloR of the clorobiocin biosynthetic gene cluster. (A) Schematic presentation of the gene inactivation experiment. thio, thiostreptone resistance gene. (B) Southern blot analysis of wild-type and mutants. Genomic DNA was restricted by SacII.

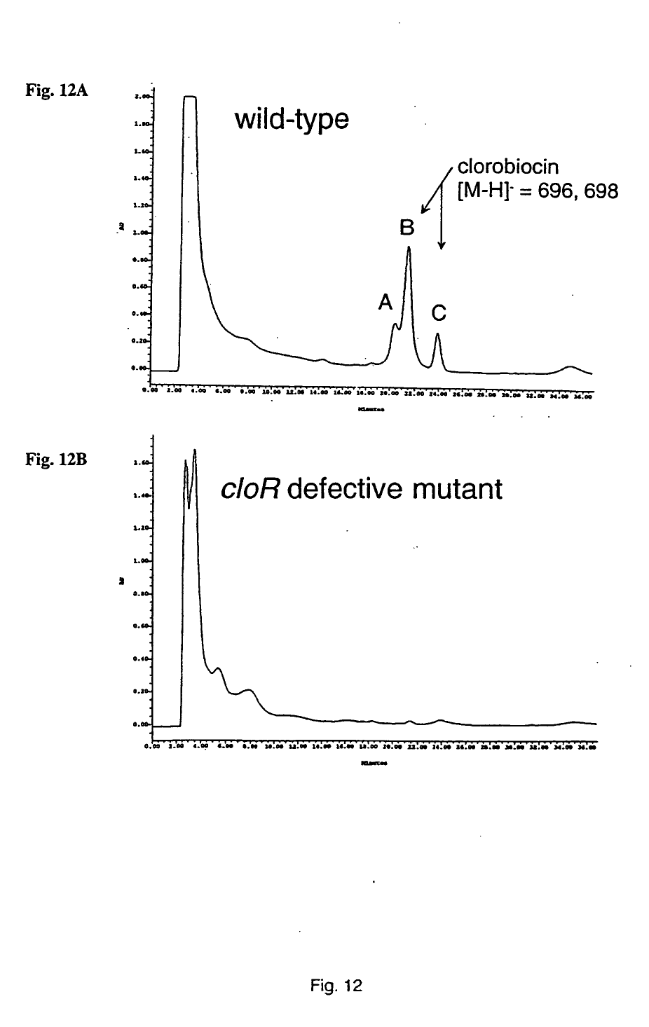

FIG. 12 shows the HPLC analysis of secondary metabolites produced by Streptomyces roseochromogenes DS 12.976 wild-type (A) and cloR defective mutant (B).

The following non-limiting examples further illustrate the present invention.

### EXAMPLES

### Example 1

### Coumermycin Biosynthetic Gene Cluster

Materials and Methods

Bacterial Strains, Plasmids and Culture Conditions

The employed bacterial strains and plasmids used for coumermycin biosynthetic gene cluster experiments are listed in Table 1. Streptomyces rishiriensis DSM 40489 was cultivated at 28° C. and 175 rpm for 24 days in baffled flasks. For isolation of chromosomal DNA, the organism was grown in liquid medium containing 1.0% malt extract, 0.4% yeast extract, 0.4% glucose, and 1.0 mM CaCl₂ (pH 7.3). For the preparation of protoplasts, Streptomyces rishiriensis was grown for 44-48 hrs in CRM medium, containing 10.3% sucrose, 2.0% tryptic soy broth, 1.0% MgCl₂×6 H₂O, 1.0% yeast extract, and 0.4% glycine (pH 7.0). Protoplasts were prepared as described by Steffensky et al. (2000) and regenerated on R2YE medium (Hopwood et al. (1985) Genetic manipulation of Streptomyces—a laboratory manual. The John Innes Foundation, Norwich, UK).

For production of coumermycin and other secondary metabolites, wild-type and mutant strains of Streptomyces rishiriensis were cultured in 500 ml baffled flasks containing 100 ml of production medium (Kawaguchi et al. (1965) J. Antibiotics, Ser A 18: 1-10) containing 3.5% Pharma Media (Hartge Ingredients GmbH & Co. KG, Hamburg, Germany), 3.0% glucose, 0.8% CaCO₃, 1.0% KH₂PO₄, 0.2% yeast extract, 0.2% KCl and 0.4% glycerol, at 28° C. and 175 rpm for 7 to 10 days. For the cultivation of integration mutants the medium was supplemented with 10 μg/ml neomycin.

Escherichia coli XL1 Blue MRF′ and ET 12567 were grown in liquid Luria-Bertani (LB) medium or on solid LB medium (1.5% agar) at 37° C. (Sambrook et al. (1989) Molecular cloning: a laboratory manual. 2^{nd} ed., Cold Spring Harbor Laboratory Press, Cold Spring Harbor, N.Y., USA).

Apramycin (100 μg/ml), carbenicillin (50 μg/ml) or neomycin (10 μg/ml for liquid media and 20 μg/ml for solid media, respectively) were used for selection of recombinant strains.

DNA Isolation and Manipulation

Standard methods for DNA isolation and manipulation were performed as described by Hopwood et al. (1985) and Sambrook et al. (1989). DNA fragments were isolated from agarose gel using the Qiagen QIAEX II Gel Extraction Kit (Qiagen, Hilden, Germany). Isolation of cosmids and plasmids was carried out with ion exchange columns (Nucleobond AX Kits, Macherey-Nagel, Duren, Germany) according to the manufacturer's protocol.

Construction and Screening of the Cosmid Library

Chromsomal DNA of Streptomyces rishiriensis DSM 40489 was partially digested with Sau3A I, dephosphorylated and ligated into cosmid vector pOJ446, which had been digested with Hpal, dephosphorylated and restricted with BamHI. The ligation products were packaged with Gigapack III XL (Stratagene, Heidelberg, Germany) and transduced into E. coli XL1 Blue MRF′.

A probe containing part of the dTDP-glucose 4,6-dehydratase gene novT from the novobiocin producer Streptomyces spheroides was prepared. An additional probe (271 bp) was prepared by PCR from the novobiocin resistance gene gyrB^{R} of Streptomyces spheroides, using primers R1 (GACGGCTCCATCTTCGAGAC) (SEQ-ID-No.8) and R2 (CGTCGGCGGCGATGGTGAC) (SEQ-ID-No.9). For hybridization experiments, the probes were labeled with DIG high prime DNA labeling and detection start kit II (Roche Molecular Biochemicals, Mannheim, Germany).

DNA Sequencing and Computer Assisted Sequence Analysis

Restriction fragments of approximately 300-3000 bp from cosmids 4-2H and 4-7D were subcloned into pBluescript SK (−). Sequencing was performed by the dideoxynucleotide chain termination method on a LI-COR automatic sequencer (MWG-Biotech AG, Ebersberg, Germany).

The DNASIS software package (version 2.1, 1995; Hitachi Software Engineering, San Bruno, Calif., USA) and the BLAST program (release 2.0) were used for sequence analysis and for homology searches in the GenBank database, respectively.

Construction of the Vector for Insertional Gene Inactivition

The vector pZK4 for couN4 (proB) disruption was constructed by insertion of the neomycin resistance gene aphll into the sequence of couN4 (proB) as follows: The aphll gene was obtained as a 0.99 kb EcoRI-HindIII fragment from plasmid pNeo4 (Table 1) and ligated into same sites of the pZW331 (Table 1), which contained the 5′ region of couN4 (proB), to give plasmid pZW2. The 3′ region of couN4 (proB) was obtained as a 0.96 kb HindIII-Xhol fragment from pZW32 (Table 1) and ligated into same sites of the pZW2, resulting in plasmid pZW3. A 2.65 kb Pstl fragment of the pZW3, containing the disrupted couN4 (proB) gene, was cloned into the same sites of pBluescript SK (−) to give the inactivation vector pZK4. In pZK4, the aphll gene fragment had the same orientation as the couN4 (proB) gene and the opposite orientation as the bla resistance gene of the vector.

Transformation of Streptomyces rishiriensis DSM 40489

Transformation of S. rishiriensis with pZK4 was carried out by polyethylene glycol (PEG)-mediated protoplast transformation. 2 g mycelia of S. rishiriensis were incubated in 7 ml P-buffer (Hopwood et al., 1985) containing 1 mg/ml lysozyme for 20-40 min at 30° C. For transformation, pZK4 was propagated in E. coli ET12567 (MacNeil et al. (1992) Gene 111: 61-68), and the resulting double-stranded plasmid DNA was denaturated by alkaline treatment (Oh et al. (1997) J. Bacteriol. 179: 122-127). The denaturated DNA (10-20 μg) was mixed with 10⁹ S. rishiriensis protoplasts (200 μl) under addition of 500 μl T buffer (Hopwood et al., 1985) containing 25% [w/v] polyethylene glycol 1000 (Roth, Karlsruhe, Germany). The resulting suspension was plated on R2YE plates. After 16-20 h at 25° C., plates were overlaid with 3 ml of soft nutrient agar containing neomycin (33.3 μg/ml) for selection of integration mutants.

Determination of the Production of Coumermycin A₁ and Other Secondary Metabolites in Streptomyces rishiriensis

Metabolites of Streptomyces rishiriensis were analyzed by a modified method of Kawaguchi et al. (1965) and Berger et al. (J. Chromatogr. Library. (Antibiotics isol., prep. and purif.) 15:101-158, 1978).

100 ml of bacterial cultures in coumermycin production medium (see above) were adjusted to pH 5 by addition of formic acid and centrifuged. The pellet was extracted with 50 ml of a mixture of acetone and 1,4-dioxane (10:1) at room temperature for 2 hrs under stirring. After filtration, the supernatant was evaporated, and the residue was dissolved in 20 ml 1 N ammonium hydroxide (pH 9). The solution was washed twice with an equal volume of ethyl acetate. The aqueous phase was adjusted to pH 5 by addition of formic acid and extracted twice with an equal volume of ethyl acetate. The ethyl acetate phase was evaporated and the residue was dissolved in 0.5 ml 4 N ammonium hydroxide in methanol. 6 μl of this solution were applied to a TLC plate (silica gel 60 F₂₅₄, E. Merck, Darmstadt, Germany). The plate was developed with dichloromethane-methanol-formic acid (45:2:1). Spots were visualized by spraying with 10% of fresh ferric chloride-potassium ferricyanide (1:1).

TABLE 1
Bacterial strains and plasmids

| Strain/plasmid | Relevant characteristics | Source/reference |
|---|---|---|
| Strains | | |
| S. rishiriensis | Coumermycin A₁ producer | DSM, |
| DSM 40489 | | Braunschweig, |
| | | Germany |
| S. rishiriensis | couN4 (proB) disrupted mutant of S. rishiriensis | this invention |
| ZW20 | DSM 40489 | |
| S. rishiriensis | couN4 (proB) disrupted mutant of S. rishiriensis | this invention |
| ZW21 | DSM 40489 | |
| E. coli XL1 | | Stratagene, |
| Blue MRF′ | | Heidelberg, |
| | | Germany |
| E. coli | DNA methylase negative strain | MacNeil et al, |
| ET12567 | | 1992 |
| Plasmids | | |
| pOJ446 | Cosmid vector | Bierman et al. |
| | | (1992) Gene 116: |
| | | 43-49 |
| pBluescript | Cloning vector | Stratagene, |
| SK(−) | | Heidelberg, |
| | | Germany |
| pWHM249 | aphll neomycin resistance gene, with the | Lomovskaya et al. |
| | Xhol site eliminated | (1997) Microbiol. |
| | | 143: 875-883 |
| pNeo4 | aphll gene; the Pstl and BamHI sites were | This invention |
| | eliminated by restriction of pWHM249 with | |
| | the respective enzyme, blunting with DNA | |
| | polymerase I and religation. | |
| pZW331 | 0.988 kb Notl-EcoRI fragment (position | This invention |
| | 11700 to 12687 in sequence AF235050), | |
| | cloned into the same sites of pBluescript | |
| | SK (−). The insert contains the 5′ region of | |
| | couN4 (proB) | |
| pZW32 | 2.844 kb EcoRI-Bg/II fragment (position | This invention |
| | 12687 to 15530 in sequence AF235050), | |
| | cloned into pBluescript SK (−), which had | |
| | been digested with EcoRI and BamHI. | |
| | The insert contains the 3′ region of couN4 | |
| | (proB). | |
| pZW2 | A 0.99 kb EcoRI-HindIII fragment, | This invention |
| | containing aphII gene from pNeo4, was | |
| | cloned in same sites of pZW331. | |
| pZW3 | A 0.96 kb HindIII-Xhol fragment from | This invention |
| | pZW32 was cloned in same sites of | |
| | pZW2. | |
| pZK4 | A 2.65 kb Pstl fragment from pZW3 was | This invention |
| | cloned in pBluescript SK(−). The | |
| | orientation of couN4 (proB) and aphll was | |
| | identical, but opposite to that of bla. | |

Cloning of the Coumermycin A₁ Biosynthetic Gene Cluster

In order to clone the coumermycin A₁ biosynthetic gene cluster, genomic DNA of the coumermycin producer, Streptomyces rishiriensis DSM 40489, to Southern hybridization with a probe of a dTDP-glucose 4,6-dehydratase gene. A single hybridizing band was detected. Likewise, hybridization with a probe from the novobiocin resistance gene gyrB^{R} encoding a novobiocin-insensitive gyrase B subunit (Thiara et al. (1993) Mol. Microbiol. 8: 496-506), resulted in a single hybridization band. Therefore, a cosmid library of the coumermycin producer was established in vector pOJ446 and screened with both probes.

The hybridizing cosmids were mapped by conventional restriction mapping as well as by hybridization of partial digests of the cosmids to pOJ446 vector sequences flanking the cosmid insert (Redenbach et al. (1998) J. Bacteriol. 180: 2796-2799).

In total, four different but overlapping cosmids were identified which extended over a continuous 89 kb region of the Streptomyces rishiriensis DSM 40489 chromosome.

Sequencing of the Cosmids and Identification of Open Reading Frames

From a core region of 30.8 kb and 35.4 kb, respectively, both strands were sequenced. This revealed the presence of 28 and 32, respectively, complete open reading frames upstream of the aminocoumarin resistance gene gyrB^{R} (FIGS. 1A and 1B, respectively).

The cluster showed striking similarity to the novobiocin biosynthetic gene cluster 15 of the identified ORFs were found to have, on average, 84% identity to corresponding ORFs of the novobiocin cluster on the amino acid level (see Table 2 and FIGS. 6A and 6B, respectively), and all of these ORFs were arranged in both clusters in identical order (see FIGS. 6A and 6B). Table 2 shows the homologies found between the genes of the coumermycin A₁ and the novobiocin cluster, as well as homologies to other GenBank entries.

For the genes contained in the novobiocin cluster, a detailed discussion of their sequence homologies and their deduced function is given in Steffensky et al. (2000). couN3, N4 and N5 (proA, proB and proC), for which no corresponding genes exist in the novobiocin cluster, show sequence similarities to pltE, pltF and pltL, respectively. These genes are supposedly involved in the conversion of proline to pyrrole-2-carboxylic acid in pyoluterorin biosynthesis in Pseudomonas fluorescens Pf-5 (Nowak-Thompson et al. (1999) J. Bacteriol. 181: 2166-2174). CouN4 (ProB), like PltF, belongs to the superfamily of the adenylate-forming enzymes and has been suggested to convert proline into an activated intermediate via an adenylation reaction. PltE, like CouN3 (ProA), shows homologies to flavine-dependent acyl-coenzyme. A dehydrogenases and is thought to catalyze the Δ^{2,3}-dehydrogenation of an activated derivative of proline. The small open reading frames couN5 (proC) and pltL (encoding 89 and 88 amino acids, respectively) show similarity to genes for acyl carrier proteins. Since coumermycin A₁ contains two pyrrole-2-carboxylic acid moieties (see FIG. 5) similar to the one found in pyoluteorin, it appears that the genes couN3, N4 and N5 (proA, proB and proC), are involved in the biosynthesis of this unit. couN2 (cumJ), immediately upstream of couN3 (proA), shares homology with dpsC, which encodes an enzyme with acyltransferase activity (Bao et al. (1999) Biochemistry 38: 9752-9757). Therefore couN2 (cumj) was assigned to the transfer of the postulated activated pyrrole-2 carboxylic acid moieties to the 3-OH of the deoxysugar moieties (see FIG. 8).

10 further open reading frames were identified in the coumermycin A₁ cluster for which no corresponding genes had been found in the novobiocin cluster. Table 2 shows the homologies between these genes and other GenBank entries. CumS, which differed from all other genes by its orientation in the cluster, showed homology to pur8, which confers resistance to puromycin (Tercero et al. (1993) Eur. J. Biochem. 218: 963-971). Therefore, cumS may be involved in resistance mechanisms.

TABLE 2
Identified open reading frames in the biosynthetic gene cluster of
coumermycin A₁ (see FIGS. 1B and 1C). nov-genes are
from the novobiocin biosynthetic gene cluster of Streptomyces
spheroides (Steffensky et al., 2000; cf. also FIG. 4).

| | | | | | Nucleotide |
|---|---|---|---|---|---|
| | Size | | | | sequence |
| | (amino | | % Identity | | accession |
| ORF | acids) | Similar to | of product | Origin | no. |
| couG | 319 | novG (318 aa) | 77 | | |
| (cumA) | | Regulator protein | 44 | Streptomyces | P08076 |
| | | (StrR) | | griseus | |
| cou Y | 71 | MbtH | 61 | Mycobacterium | O05821 |
| (cum B) | | | | tubercolosus | |
| couH | 599 | novH (600 aa) | 80 | | |
| (cumC) | | Peptide synthetase | 46 | Amycolatopsis | AJ223998 |
| | | | | orientalis | |
| coul | 407 | novl (407 aa) | 88 | | |
| (cumD) | | Cytochrome P-450 | 39 | Streptomyces | AJ250199 |
| | | (NikQ) | | tendae TU901 | |
| CouJ | 258 | novJ (262 aa) | 84 | | |
| (cumE) | | 3-Ketoacyl-[ACP] | 45 | Vibrio harveyi | P55336 |
| | | reductase | | | |
| couK | 245 | novK (244 aa) | 76 | | |
| (cumF) | | Reductase | 32 | Klebsiella | Q04520 |
| | | | | terrigena | |
| couL | 529 | novL (527 aa) | 80 | | |
| (cumG) | | Acyl-CoA | 39 | Streptomyces | AL049763 |
| | | synthetase | | coelicolor | |
| couM | 402 | novM (379 aa) | 79 | | |
| (cumH) | | Glycosyl | 44 | Streptomyces | AF077869 |
| | | transferase | | argillaceus | |
| couN1 | 95 | Hypothetical | | | |
| (cumI) | | protein | | | |
| couN2 | 355 | Acyltransferase | 38 | Streptomyces | L35560 |
| (cumJ) | | (DpsC) | | peucetius | |
| couN3 | 373 | Acyt-CoA | 46 | Pseudomonas | AF081920 |
| (proA) | | dehydrogenase | | fluorescens Pf-5 | |
| | | (PltE) | | | |
| couN4 | 501 | Acyl-CoA | 51 | Pseudomonas | AF081920 |
| (proB) | | synthetase (PltF) | | fluorescens Pf-5 | |
| couN5 | 89 | PltL | 38 | Pseudomonas | AF081920 |
| (proC) | | | | fluorescens Pf-5 | |
| couN6 | 399 | Hypothetical | | | |
| (cumK) | | protein | | | |
| couN7 | 281 | Hydrolase | 31 | Pseudomonas | AF090329 |
| (cumL) | | | | fluorescens DSM | |
| | | | | 50106 | |
| couO | 230 | novO (230 aa) | 84 | | |
| (cumM) | | Methyltransferase | 27 | Streptomyces | S44970 |
| | | (LmbW) | | linconensis | |
| couP | 276 | novP (262 aa) | 88 | | |
| (cumN) | | O-Methyl- | 53 | Micromonospora | D16097 |
| | | transferase | | griseorubida | |
| couR1 | 474 | Hypothetical | | | |
| (cumO) | | protein | | | |
| couR2 | 377 | Decarboxylase | 36 | Streptomyces | Y14337 |
| (cumP) | | | | viridochromogenes | |
| couR3 | 302 | PduX | 34 | Salmonella | AF026270 |
| (cumQ) | | | | enterica, | |
| | | | | Salmonella | |
| | | | | typhimurium | |
| couR4 | 389 | Dehydrogenase | 39 | Neisseria | U58911 |
| (cumR) | | | | meningitidis | |
| couR5 | 491 | Resistance protein | 39 | Streptomyces | P42670 |
| (cumS) | | | | alboniger | |
| couR6 | 290 | Regulator protein | 39 | Alcaligenes | S80112 |
| (cumT) | | (TfdR) | | eutrophus | |
| couS | 288 | novS (288 aa) | 81 | | |
| (cumU) | | dTDP-4-keto-6- | 51 | Streptomyces | P29781 |
| | | deoxyhexose | | griseus | |
| | | reductase | | | |
| couT | 336 | novT (336 aa) | 89 | | |
| (cumV) | | dTDP-glucose 4,6- | 59 | Saccharopoly- | L37354 |
| | | dehydratase | | spora erythraea | |
| couU | 420 | novU (420 aa) | 85 | | |
| (cumW) | | C-Methyl- | 37 | Saccharopoly- | X60379 |
| | | transferase | | spora erythraea | |
| couV | 296 | novV (297 aa) | 90 | | |
| (cumX) | | dTDP-1-glucose | 61 | Mycobacterium | U55242 |
| | | synthase | | tuberculosis | |
| CouW | 198 | novW (207 aa) | 82 | | |
| (cumY) | | dTDP-4-keto-6- | 50 | Streptomyces | P29783 |
| | | deoxyglucose 3,5- | | griseus | |
| | | epimerase | | | |
| gyrB^{Rcou} | 677 | gyrB^{R}-nov (677 aa) | 91 | | |
| (gyrB^{R}- | | DNA gyrase B | 77 | Streptomyces | P35886 |
| cum | | | | coelicolor | |

Insertional Inactivation of the couN4 (proB) Gene in S. rishiriensis

The striking similarity between the gene cluster between the gene clusters for coumermycin and novobiocin biosynthesis made it very likely that we had indeed cloned the coumermycin biosynthetic gene cluster. Functional proof for this hypothesis was provided by an insertional gene inactivation experiment. The gene couN4 (proB), located in the central region of the cluster and possibly involved in pyrrole biosynthesis (see above) was chosen for this experiment.

An inactivation vector, pZK4, was constructed in which the structural gene couN4 (proB) was disrupted by insertion of a neomycin resistance gene (aphll, 0.99 kb; see FIG. 9). The gene was introduced into S. rishiriensis by homologous recombination. After selection for the neomycin resistant phenotype, mutant strains were analyzed by Southern hybridization. Two mutant strains, ZW20 and ZW21, were identified which showed the desired gene replacement resulting from a double crossover (FIG. 9).

Culture extracts from the wild-type strain of S. rishiriensis and from the mutant strains, ZW20 and ZW21, were analyzed by thin-layer chromatography in comparison with a coumermycin A₁ standard. While coumermycin A₁ production in the wild-type strain S. rishiriensis could be clearly detected, coumermycin A₁ production in both mutants was completely abolished. The thin-layer chromatogram showed the accumulation of a new secondary metabolite in the couN4 (proB) disrupted strains, clearly different from coumermycin A₁ (FIG. 10).

### Example 2

### Clorobiocin Biosynthetic Gene Cluster

Materials and Methods

Bacterial Strains, Plasmids, and Culture Conditions.

S. roseochromogenes var. oscitans DS 12.976 was kindly provided by Aventis and routinely cultivated at 28° C. for 2 days in HA medium containing 1.0% malt extract, 0.4% yeast extract, 0.4% glucose, and 1.0 mM CaCl₂ (pH 7.3). For production of clorobiocin and other secondary metabolites, wild-type and mutant strains of S. roseochromogenes were pre-cultured in 500 ml baffled flasks containing 50 ml medium. After growth for 48 h at 33° C. and 210 rpm, 5 ml of this pre-culture was inoculated into 500 ml baffled flasks containing 50 ml production medium (Mancy et al., 1974). In this medium, cells were cultured at 33° C. and 210 rpm for 7 days. Escherichia coli XL1 Blue MRF′ (Stratagene, Heidelberg; Germany) was grown in liquid or solid Luria-Bertani medium at 37° C. (Sambrook and Russel, 2001). SuperCos-1 was purchased from Stratagene (Heidelberg; Germany). pBSKT, an integrative vector carrying carbenicillin and thiostreptone resistances, was described by Lombo et al. (1997).

Carbenicillin (50 μg/ml) and thiostreptone (50 μg/ml) were used for selection of recombinant plasmids and strains.

Genetic Procedures

Standard methods for DNA isolation and manipulation were performed as described by Kieser et al. (2000). DNA fragments were isolated from agarose gels using a NucleoSpin 2 in 1 extraction kit (Macherey-Nagel, Düren; Germany). Isolation of cosmids and plasmids was carried out with ion-exchange columns (Nucleobond AX kit; Macherey-Nagel, Duren; Germany). Genomic DNA was isolated from Streptomyces strains by lysozyme treatment and phenol-chloroform extraction.

Construction and Screening of the Cosmid Library

Chromosomal DNA of S. roseochromogenes was partially digested by Sau3Al, dephosphorylated, and ligated into the BamHI sites of Supercos-1. The ligation products were packaged with Gigapack III XL (Stratagene, Heidelberg; Germany) and transduced into E. coli XL1 Blue MRF′.

Southern blot analysis was performed on Hybond-N membranes (Amersham, Braunschweig; Germany) with digoxigenin-labeled probes by using the DIG high prime DNA labeling and detection kit 11 (Roche Molecular Biochemicals, Mannheim; Germany). Two probes, one containing a part of the dTDP-glucose 4,6-dehydratase gene novT (Steffensky et al., 2000) and the other containing a 1.58 kb Sphl/BamHI-fragment of the novobiocic acid synthetase gene novL (Steffensky et al., 2000) were used for hybridization.

DNA Sequencing and Computer-Assisted Sequence Analysis

Double-stranded sequencing of the entire cosmid K1F2 (carrying an insert of 42, 291 bp) was performed by the dideoxynucleotide chain termination method on a LI-COR automatic sequencer (MWG-Biotech AG, Ebersberg; Germany) using a shotgun library with DNA fragments of approximately 1.5 to 2.0-kb in length.

The DNASIS software package (version 2.1; Hitachi Software EngineeRing, San Bruno; Calif.) was used for sequence analysis. Amino acid sequence homology searches were carried out in the GenBank database by using the BLAST program (release 2.0).

Construction of the Vector pFP02 for In-Frame Gene Inactivation.

For inactivation of cloR in S. roseochromogenes, the fragment cloR-1 (1282 bp) and the fragment cloR-2 (1301 bp) were amplified by PCR. The primer pairs were: cloR-1/HindIII, 5′-GTCACCGGAAGCTTTGCCTG-3′ (SEQ-ID-No. 10); cloR-1/Pstl, 5′-GCATGTTCTGCAGAGCCTTG-3′ (SEQ-ID-No.11); cloR-2/Pstl, 5′-GCCTGCACTGCAGGCCCCAA-3′ (SEQ-ID-No.12); cloR-2BamHI, 5′-TCGTAGGATCCTCCCGTCGTC-3′ (SEQ-ID-No.13). Restriction sites introduced into the sequence are underlined in the primer sequences. The amplified DNA fragment cloR-1 was digested with HindIII and Pstl and cloned into the corresponding sites of vector pBSKT, a pBluescript SK(+) derivative containing carbenicillin and thiostreptone resistances, resulting in pFP01. The PCR fragment cloR-2 was digested with Pstl and BamHI and ligated into the same sites of pFP01 to give pFP02.

Transformation of S. roseochromogenes and Selection for Recombinant Mutants.

Transformation of S. roseochromogenes with pFP02 was carried out by polyethylene glycol-mediated protoplast transformation (Kieser et al., 2000). For preparation of protoplasts, mycelia of S. roseochromogenes were grown in CRM medium, containing 10.3% sucrose, 2.0% tryptc soy broth, 1.0% MgCl₂.6H₂O, 1.0% yeast extract, and 0.75% glycine (pH 7.0), for 48 h, harvested, and incubated in 5 ml P-buffer per gram mycelia, containing 1 mg of lysozyme per ml, for 30 to 60 min at 30° C.

For transformation, pFP02 was mixed with 200 μl P-buffer containing 10⁹ S. roseochromogenes protoplasts and 500 μl T-buffer containing 50% (wt/vol) polyethylene glycol 1000 (Roth, Karlsruhe; Germany). The resulting suspension was plated on R2YE agar medium (Kieser et al., 2000). After incubation for 20 h at 30° C., the plates were overlaid with 3 ml of soft R2YE agar containing a total of 500 μg thiostreptone for selection of recombinant mutants.

Analysis of Secondary Metabolites

Bacterial culture (20 ml) was acidified to pH 4 with HCl and extracted twice with an equal volume of ethyl acetate. After centrifugation, the solvent was evaporated and the dried extract was resuspended in 1 ml methanol.

Metabolites were analysed by HPLC with a Multosphere RP18-5 column (250×4 mm, 5 μm) with a linear gradient from 60% to 100% methanol in 1% aqueous formic acid and detection at 340 nm. Authentic clorobiocin (Aventis) was used as standard.

Cloning and Sequencing of the Clorobiocin Biosynthetic Gene Cluster

The novobiocin (Steffensky et al., 2000) and coumermycin A₁ (Wang et al., 2000) biosynthetic gene clusters (FIGS. 1A-1C and 4) have been cloned by screening cosmid libraries of the producing strains with a probe for a dNDP-glucose 4,6-dehydratase gene, which is involved in the biosynthesis of the deoxysugar moiety of these antibiotics. Since clorobiocin contains the same deoxysugar moiety, the same gene (novT of the novobiocin biosynthetic gene cluster) was used as probe. The gene novL served as additional probe. NovL encodes the novobiocic acid synthetase which catalyses the formation of the amide bond between Ring A and Ring B of novobiocin (Steffensky et al., 2000). A similar enzyme is expected to be involved in clorobiocin biosynthesis. Southern hybridisations of genomic DNA of the clorobiocin producer S. roseochromogenes with these two probes each resulted in a single band.

Therefore, a cosmid library from S. roseochromogenes was constructed in SuperCos-1, and screened with the novT and novL probes. The hybridising cosmids were analysed by restriction mapping. Four different but overlapping cosmids were obtained which covered a continuous 55 kb region of the chromosome. Cosmid K1F2 was sequenced on both strands. This revealed the presence of 36 complete open reading frames (ORFs) in addition to a partial sequence of the aminocoumarin resistance gene gyrB^{R}. The first 9 ORFs in the insert of cosmid K1F2 were homologous to genes coding for primary metabolic enzymes. The next 27 ORFs, however, showed striking similarity to genes of the novobiocin and/or coumermycin A₁ biosynthetic gene cluster. Remarkably, in all three clusters all the corresponding ORFs were arranged in the same order and oriented in the same direction (FIGS. 6A and 6B).

Table 3 lists the homologies found between the genes in cosmid K1F2 and the genes of the novobiocin and coumermycin A₁ clusters, as well as homologies to other GenBank entries. The sequence of cosmid K1F2 is shown in FIG. 2A.

TABLE 3
Identified ORFs in the biosynthetic gene cluster of clorobiocin

| | size of the | | % | |
|---|---|---|---|---|
| | product | | identity | |
| | (amino | | of | accession |
| ORF | acids [aa]) | Similar entity or entities^{a} | products | number |
| ORF1 | 306 | Fkbl (lysine cyclodeaminase) | 37 | AAF86391 |
| | | from S. hygroscopicus | | |
| ORF2 | 197 | sarcosine oxidase gamma | 44 | Q46338 |
| | | subunit from Corynebacterium | | |
| | | sp. | | |
| ORF3 | 962 | sarcosine oxidase alpha | 59 | Q46337 |
| | | subunit from Corynebacterium | | |
| | | sp. | | |
| ORF4 | 93 | sarcosine oxidase delta subunit | 66 | Q46336 |
| | | from Corynebacterium sp. | | |
| ORF5 | 406 | sarcosine oxidase beta subunit | 80 | P40875 |
| | | from Corynebacterium sp. | | |
| ORF6 | 406 | serine | 75 | O86565 |
| | | hydroxymethyltransferase from | | |
| | | S. coelicolor | | |
| ORF7 | 218 | putative transcriptional | 45 | AL596248.1 |
| | | regulator from S. coelicolor | | |
| ORF8 | 149 | unknown protein | | |
| ORF9 | 78 | transposase from S. coelicolor | 68 | AL109949.1 |
| cloE | 217 | novE (217 aa) | 82 | |
| | | ImbU protein from S. | 43 | S44974 |
| | | lincolnensis | | |
| cloF | 362 | novF (362 aa) | 73 | |
| | | prephenate dehydrogenase | 30 | AAK81837 |
| | | from S. lavendulae | | |
| cloG | 319 | novG (318 aa)/couG (319 aa) | 79/80 | |
| | | regulatory protein (StrR) from | 40 | S44506 |
| | | S. glaucescens | | |
| cloY | 71 | couY (71 aa) | 81 | |
| | | hypothetical protein from S. coelicolor | 68 | T36310 |
| cloH | 600 | novH (600 aa)/couH (599 aa) | 75/80 | |
| | | peptide synthetase-like protein | 50 | AAG34184.1 |
| | | from S. antibioticus | | |
| cloI | 407 | novI (407 aa)/couI (407 aa) | 90/95 | |
| | | cytochrome P-450 enzyme from | 66 | AAK06805.1 |
| | | S. antibioticus | | |
| cloJ | 258 | novJ (262 aa)/couJ (258 aa) | 72/77 | |
| | | 3-oxoacyl reductase from bacillus | 48 | BAB06210 |
| | | halodurans | | |
| cloK | 245 | novK (244 aa)/couK (245 aa) | 77/81 | |
| | | reductase from S. antibioticus | 35 | AAG34182 |
| cloL | 527 | novL (527 aa)/couL (529 aa) | 86/86 | |
| | | amide synthetase from S. antibioticus | 37 | AAG34183 |
| cloM | 390 | novM (379 aa)/couM (402 aa) | 78/78 | |
| | | glycosyltransferase from S. argillaceus | 38 | AAC64927 |
| cloN1 | 95 | couN1 (95 aa)/hypothetical | 86 | |
| | | protein | | |
| cloN2 | 355 | couN2 (355 aa) | 86 | |
| | | dpsC from S. peucetius | 32 | 1093565 |
| cloN3 | 376 | couN3 (373 aa) | 81 | |
| | | acyl-CoA dehydrogenase (pltE) | 45 | AAD24879 |
| | | from Pseudomonas fluorescens | | |
| cloN4 | 501 | couN4 (501 aa) | 87 | |
| | | acyl-CoA synthetase (pltF) from | 45 | AAD24881 |
| | | Pseudomonas fluorescens | | |
| cloN5 | 89 | couN5 (89 aa) | 91 | |
| | | hypothetical protein (pltL) from | 34 | T17418 |
| | | Pseudomonas fluorescens | | |
| cloN6 | 561 | couN6 (560 aa) | 94 | |
| | | hypothetical protein | | |
| cloN7 | 278 | couN7 (281 aa) | 82 | |
| | | putative hydrolase from S. coelicolor | 34 | CAB95984 |
| clo-hal | 524 | nonheme halogenase from S. lavendulae | 35 | AAK81830 |
| cloP | 277 | novP (262 aa)/couP (276 aa) | 91/89 | |
| | | O-methyltransferase III from S. olivaceus | 58 | CAC32469 |
| cloQ | 324 | novQ (271 aa)/hypothetical | 80 | |
| | | protein | | |
| cloR | 277 | novR (270 aa) | 95 | |
| | | putative aldolase from S. coelicolor | 47 | CAB82026 |
| cloS | 288 | novS (288 aa)/couS (288 aa) | 84/87 | |
| | | 4-ketoreductase from S. antibioticus | 50 | AAD55455 |
| cloT | 336 | novT (336 aa)/couT (336 aa) | 82/87 | |
| | | dNDP-glucose-4,6-dehydratase | 66 | AAF13998 |
| | | from S. globisporus | | |
| cloU | 420 | novU (420 aa)/couU (420 aa) | 88/90 | |
| | | D-mycarose 3-C- | 48 | CAB96549 |
| | | methyltransferase from | | |
| | | S. argillaceus | | |
| cloV | 296 | novV (297 aa)/couV (296 aa) | 89/92 | |
| | | dTDP-glucose synthase from | 74 | AAD31796 |
| | | S. spectabilis | | |
| cloW | 198 | novW (207 aa)/couW (198 aa) | 86/91 | |
| | | dTDP-4-keto-6-deoxyglucose 3,5- | 54 | S44236 |
| | | epimerase from S. glaucescens | | |
| cloZ | 253 | hypothetical protein | | |
| gyrB^{R} | partial sequence | gyrB^{R}-nov (novobiocin cluster)/ | | |
| | | gyrB^{R}-cou (coumermycin cluster) | | |

^{a}nov genes are from the novobiocin biosynthetic gene cluster of S. spheroides cou genes are from the coumermycin A₁ biosynthetic gene cluster of S. rishiriensis

Generation of a cloR Defective Mutant

In order to test whether cloR was indeed involved in Ring A biosynthesis, a gene inactivation experiment was carried out. An inactivation vector carrying a thiostreptone resistance gene (pFP02) was constructed in which the structural gene cloR was disrupted by in-frame deletion (FIG. 11). After transforming S. roseochromogenes protoplasts with plasmid pFP02, thiostreptone-resistant colonies were obtained. Southern blotting confirmed that pFP02 had been integrated via a single cross-over recombination event (FIG. 11). The single cross-over mutant RSCO2 was grown in the absence of thiostreptone, sporulated, and examined for loss of resistance as consequence of double cross-over events. Several sensitive colonies were obtained. Two mutants, named RDCO30 and RDCO32, were further examined. Chromosomal DNA from S. roseochromogenes wild-type as well as from mutants RSCO2, RDCO30 and RDCO32 was digested by SaclI and hybridised with a probe containing a part of the cloR gene. A band at 1.1 kb was detected in the S. roseochromogenes wild-type, while chromosomal DNA from mutant RDCO30 showed the expected band of 2.2 kb corresponding to the in-frame deletion of cloR (FIG. 11). This strain was subsequently cultured and examined by HPLC for the production of clorobiocin. As shown in FIG. 12B, the production of clorobiocin was abolished in this mutant. The other sensitive strain, RDCO32, represented a reversion to the wild-type, as shown in Southern blots (band at 1.1 kb). Correspondingly, clorobiocin production in this strain was identical to that in the wild-type strain (FIG. 12A).

Summary: Comparison of Aminocoumarin Biosynthetic Gene Clusters

The coumarin antibiotics are closely related to each other in their chemical structure (FIG. 5). The biosynthetic gene cluster of novobiocin has recently been identified (Steffensky et al., 2000; see FIG. 4). Other coumarin antibiotics such as coumermycin A₁, are of special pharmaceutical interest due to their pronounced antibacterial activity and its extremely high affinity to bacterial gyrase (see, e.g. Peng et al., 1993; Ryan (1979) in: Hahn (ed.), Antibiotics: mechanism of action of antibacterial agents. vol. V(1): 214-234, Springer Verlag, Berlin, Germany).

The complete biosynthetic gene clusters of coumermycin A₁ and clorobiocin as well as the aminocoumarin part of the biosynthetic cluster of simocyclinone were cloned and sequenced. The clusters showed striking similarity to the novobiocin biosynthetic gene cluster (FIGS. 6A and 6B). In the clusters, nearly all of the open reading frames are oriented into the same direction. At the 3′ end of each of the clusters of novobiocin, coumermycin A₁ and clorobiocin, a gene encoding for a coumarin-resistant gyrase B subunit is located. This gene has been described previously as the principal novobiocin resistance gene in the novobiocin producer Streptomyces spheroides (Thiara et al. (1993) Mol. Microbiol. 8: 495-506), and expression of the gyrB^{R} gene from the coumermycin cluster in Streptomyces lividans showed that it also conferred resistance to both novobiocin and coumermycin A₁.

Immediately upstream of the resistance gene, the clusters of novobiocin, coumermycin A₁ and clorobiocin contain five highly homologous genes in exactly identical order (couS, T, U, V, W (cumU, V, W, X, Y, novS, T, U, V, W and cloS, T, U, V, W, respectively). Based upon their homology to known genes of deoxysugar biosynthesis, these genes were assigned to the first five steps required for the biosynthesis of the deoxysugar moiety of novobiocin (see FIG. 5 and FIG. 7), and functional proof for this hypothesis was given by an inactivation experiment with novT (Steffensky et al., 2000). Coumermycin A₁ contains the same deoxysugar moiety as novobiocin, and in the coumermycin A₁ biosynthetic gene cluster exactly the same deoxysugar biosynthetic genes were found. This provides additional support to the functional assignment of these genes (FIGS. 6A and 6B).

One of the last steps of novobiocin biosynthesis is the O-methylation of the 4-OH of the deoxysugar moiety, and the gene novP has been assigned to this reaction. The same O-methylation is required in coumermycin A₁ and clorobiocin biosynthesis, and indeed a gene homologous to novP, i.e. couP (cumN), i.e. cloP was found at the corresponding position of the coumermycin and clorobiocin cluster, respectively. The gene novO, immediately upstream of the novP, had been assigned to the C-methylation reaction at position 8 of the coumarin ring of novobiocin, and this assignment is now supported by the presence of a highly homologous gene, couO (cumM), likely to carry out the same reaction in coumermycin biosynthesis.

Attachment of the deoxysugar to the 7-OH of the aminocoumarin ring requires very similar glycosyl transferases in novobiocin, coumermycin and clorobiocin biosynthesis, and indeed three very similar glycosyl transferase genes, novM, couM (cumH) and cloM, are found at the same relative position of both clusters.

In novobiocin (FIG. 5), the aminocoumarin and the substituted benzoate ring are linked by an amide bond, and the gene novL has been functionally identified, by overexpression and purification, as the amide synthetase responsible for both adenylation of the substituted benzoyl moiety and its transfer onto the amino group. In the structure of coumermycin, two corresponding amide bonds are present, linking the two aminocoumarin rings to a central 3-methylpyrrole-2,4-dicarboxylic acid moiety. The gene couL (cumG) shows high homology to novL and is located at the same relative position of the gene cluster. It is most probably involved in the formation of these amide bonds.

The gene couH (cumC) and cloH displays distinct homology to peptide synthetases, and its deduced amino acid sequence shows the presence of the typical conserved motifs of peptide synthetases described by Marahiel et al. (Chem. Rev. 97: 2651-2673, 1997), including the 4-phosphopantetheinyl attachment site required for covalent binding of the acyl substrate in form of a thioester. couH (cumC) and cloH are very similar to novH, which is located at the same relative position of the novobiocin cluster. NovH has been proven not to be involved in the formation of the amide bond between the aminocoumarin ring and the substituted benzoate ring of novobiocin. NovH may catalyze the activation of tyrosine, or a derivative thereof, during the biosynthesis of one of the two aromatic rings of novobiocin. The presence of a very similar gene in the cluster for coumermycin, which contains the aminocoumarin but not the substituted benzoate ring, suggests that novH and the corresponding couH (cumC) are involved in the biosynthesis of the aminocoumarin ring found in both antibiotics.

Immediately downstream of couH (cumC), the gene couI (cumD) is found which shows homology to cytochrome P-450 enzymes: the very recently described NikQ catalyzes the β-hydroxylation of histidine during nikkomycin biosynthesis (Lauer et al. (2000) Eur. J. Biochem. 267: 1698-1706); and ORF20 of the chloroeremomycin biosynthetic gene cluster (Van Wageningen et al. (1998) Chem. Biol. 5: 155-162) may be involved in the β-hydroxylation of tyrosine (Lauer et al., 2000). The biosynthesis of the aminocoumarin moieties of novobiocin and coumermycin, which are derived from tyrosine (Kominek et al. (1974) Dev. Ind. Microbiol. 15: 60-69; Li et al. (1998) Tetrahedron Lett. 39: 2717-2720), requires the introduction of an oxygen at the β-position of tyrosine. The cytochrome P-450 enzyme CouI (CumD), and the corresponding NovI of the novobiocin cluster, are candidates for the catalysis of this reaction.

The ring oxygen of the aminocoumarin moiety of novobiocin has been shown to derive from the carboxy group of tyrosine, rather than from molecular oxygen (Bunton et al. (1963) Tetrahedron 19: 1001-1010), suggesting a formation of the aminocoumarin ring by a unique oxidative cyclization mechanism rather than by orthohydroxylation of tyrosine followed by simple lactonization. The formation of the aminocoumarin ring may therefore require oxidation of a (hypothetical) β-hydroxy-tyrosine derivative to a β-keto-tyrosine derivative and a subsequent oxidative cyclization. CouJ (CumE) (and the corresponding NovJ) show homology to 3-ketoacyl-[acyl carrier protein]-reductase, and may catalyze the first of the two oxidation steps. The adjacent CouK (CumF), and the corresponding NovK, share homology to redox enzymes and may possibly catalyze the oxidative cyclization step.

The genes for the biosynthesis of the characteristic aminocoumarin ring of the aminocoumarin antibiotics must be present in all of the aminocoumarin clusters, and a comparison of the clusters is therefore an obvious method to identify possible candidate genes for the biosynthesis of this moiety.

This comparison leads to the finding that the gene products of couH, I, J and K (cumC, D, E and F) and cloH, I, J and K corresponding to novH, I, J and K, may catalyze the formation of the aminocoumarin ring in a reaction sequence as shown in FIG. 8, i.e. activation of tyrosine and covalent binding in form of a thioester, β-hydroxylation, oxidation to a β-keto-acyl-intermediate, and oxidative cyclization. It is noteworthy that also in the chloroeremomycin cluster a gene with homology to a peptide synthetase, ORF19, is situated immediately upstream of the P-450 gene ORF20, suggesting that β-hydroxylation may require prior activation of tyrosine also in the biosynthesis of chloroeremomycin. Likewise, β-hydroxylation of free histidine could not be demonstrated after heterologous expression of the P-450 enzyme NikQ mentioned above, and the involvement of an additional enzyme was postulated (Lauer et al., 2000).

NovN has been suggested to catalyze the transfer of the carbamoyl group to the deoxysugar moiety of novobiocin (FIG. 5). Coumermycin does not contain this carbamoyl group, and no gene with similarity to novN is found in the coumermycin cluster. However, at the same relative position the gene couN2 (cumJ) is found which shows homology to acyltransferases and which appears to catalyse the transfer of the pyrrolcarboxylic acid moieties to the deoxysugar moieties of coumermycin A₁. Immediately downstream of couN2 (cumJ), the genes couN3, N4 and N5 (proA, B, and C) are found which share homology to genes supposedly involved in the conversion of proline to Pyrrole-2-carboxylic acid in the biosynthesis of pyoluteorin in Pseudomonas fluorescens Pf-5 (Nowak-Thompson et al. (1999) J. Bacteriol. 181: 2166-2174). These genes are assigned to the biosynthesis of the pyrrole-2-carboxylic acid moieties of coumermycin A₁ (FIG. 8). An incorporation of proline into coumermycin A₁ has been demonstrated previously (Scanell et al. (1970) Antimicrob. Agents Chemother. 1969: 139-143).

The disclosure of each of the scientific articles, books, manuals or other references referred to herein is incorporated by reference herein.

1. An isolated nucleic acid having a nucleotide sequence coding for at least one enzyme or a functionally active fragment thereof encoded by an aminocoumarin biosynthetic gene duster, wherein said aminocoumarin is selected from the group consisting of coumermycin A1, clorobiocin—and simocyclinone.

2. The nucleic acid according to claim 1 wherein said nucleotide sequence comprises at least one open reading frame (ORF) contained in the nucleotide sequences shown in SEQ-ID-No. 1 to 7.

3. The nucleic acid according to claim 1 wherein said nucleotide sequence codes for a mutant enzyme comprising a substitution, addition, insertion and/or deletion of one or more amino acid(s) in comparison to its wild type sequence.

4. The nucleic acid according to claim 3 wherein said mutant is composed of a mixture of amino acid sequences encoded by one or more genes contained in said aminocoumarin biosynthetic clusters.

5. The nucleic acid according to anyone of claims 1 to 4 wherein said nucleotide sequence codes for more than one enzyme or a functionally active fragment thereof.

6. The nucleic acid according to claim 5 wherein the enzymes or functionally active fragments thereof are encoded by different aminocoumarin biosynthetic gene clusters.

7. A vector containing at least the nucleic acid according to anyone of claim 1.

8. A host organism containing the nucleic acid according to anyone of claim 1 or the vector according to claim 7.

9. A polypeptide encoded by an ORF of an aminocoumarin biosynthetic gene cluster, wherein said aminocoumarin is selected from the group consisting of coumermycin A₁, clorobiocin and simocyclinone.

10. A method for the production of a modified aminocoumarin selected from the group consisting of novobiocin, clorobiocin, coumermycin A₁ and simocyclinone,—said method comprising the steps of:
(a) inactivating at least one gene of the gene cluster for the biosynthesis of one aminocoumarin in an organism containing said gene cluster,
(b) introducing into said organism at least one biosynthetic gene from another organism and/or feeding said organism with an analogue of a structural moiety of an aminocoumarin;
(c) cultivating said organism containing said gene cluster in a suitable medium; and
(d) isolating the aminocoumarin produced by said organism.

11. The method according to claim 10, wherein said at least one biosynthetic gene from another organism is a gene of another aminocoumarin biosynthetic gene cluster.

12. The method according to claims 10 or 11, wherein said organism containing said gene cluster is selected from the group of consisiting of S. spheroides, S. niveus, S. roseochromogenes, S. rishiriensis and S. antibioticus.

13. An aminocoumarin compound substantially being composed of structural elements derived from different aminocoumarins selected from the group consisting of novobiocin, clorobiocin, coumermycin A₁ and simocyclinone.

14. A pharmaceutical composition containing the aminocoumarin compound according to claim 13 in a pharmaceutically effective amount, optionally in combination with a pharmaceutically acceptable carrier and/or diluent.

15. The pharmaceutical composition according to claim 14 for the treatment of infections with gram-positive bacteria and of malignant diseases.

16. The pharmaceutical composistion according to claim 14 further containing a pharmaceutically effective amount of a cytostatic agent.

17. The pharmaceutical composition according to claim 16 wherein said cytostatic agent is selected from the group consisting of podophyllotoxin derivatives.

18. A method for treating a patient comprising the step of administering to said patient a pharmaceutically effective amount of the aminocoumarin compound according to claim 13.
